(19) United States
(12) Patent Application Publication
(10) Pub. No.: US 2026/0162772 A1
(43) Pub. Date: Jun. 11, 2026

(54) MICROBIOME BASED DETECTION OF ENDOMETRIOSIS

(71) Applicant: HerAnova Lifesciences Inc., Burlington, MA (US)

(72) Inventors: Farideh Bischoff, Houston, TX (US); Wing Hing Wong, Menlo Park, CA (US); Yanqin Yu, Hangzhou (CN); Jiaying He, Hangzhou (CN); Xiaochun Xu, Hangzhou (CN)

(21) Appl. No.: 19/403,211

(22) Filed: Nov. 27, 2025

Related U.S. Application Data
(60) Provisional application No. 63/726,260, filed on Nov. 28, 2024.

(51) Int. Cl. G16B 40/20; A61K 31/57; A61K 45/06; C12Q 1/689

(52) U.S. Cl. CPC G16B 40/20; A61K 31/57; A61K 45/06; C12Q 1/689; C12Q 2600/118; C12Q 2600/158; C12Q 2600/178

(57) ABSTRACT

Provided herein are methods of assessing whether a subject has endometriosis or is predisposed to endometriosis based on microbiome-based biomarkers in a sample of the subject. Kits useful for performing such assessments are also provided. Methods of treating or preventing endometriosis guided by information obtained from methods disclosed herein are also provided.

This application claims priority to U.S. Provisional Application No. 63/726,260, filed Nov. 28, 2024, which is incorporated herein by reference in its entirety.

### 1. REFERENCE TO SEQUENCE LISTING SUBMITTED ELECTRONICALLY

This application incorporates by reference a Sequence Listing as an XML file entitled “522A003WO02_SL.XML” created on Nov. 27, 2025 and having a size of 50,915 bytes.

### 2. FIELD

The present invention relates to the field of molecular biology, cell biology, physiology and pathology.

### 3. BACKGROUND

Endometriosis affects 10-15% of women of reproductive age and 20-50% of infertile women. Although most women with endometriosis report the onset of symptoms during adolescence, many of them experience a delay of 7-10 years in the diagnosis, which can result in unnecessary suffering and reduced quality of life. The current standard of diagnosis is laparoscopic visualization and subsequent histological confirmation, and the surgical nature of laparoscopy usually results in the delay in diagnosis. As early diagnosis and treatment can mitigate pain and prevent disease progression, means to detect endometriosis at early onset represents unmet needs.

Methods and systems provided herein address these needs and provide related advantages.

### 4. SUMMARY

The present disclosure provides non-invasive methods and systems for characterizing a microbiome to assess a likelihood of endometriosis in a subject. The present disclosure integrates high-throughput genomic analysis with advanced computational modeling to detect phase-specific microbial signatures associated with the disease. In some embodiments, the method comprises obtaining a dataset representing a plurality of nucleic acid sequences derived from a sample from the subject; quantifying the relative abundance of a specific panel of bacterial taxa; calculating a Functional Dysbiosis Score (FDS) based on the balance between protective Lactobacillus species and pathogenic taxa; and processing these features using a trained machine learning classifier to generate a classification output indicating the presence or absence of endometriosis.

In some embodiments, methods provided herein comprise the stratification of diagnostic panels based on the subject's menstrual cycle phase. In embodiments where the sample is obtained during the proliferative phase, the panel of bacterial taxa comprises at least one taxon selected from the group consisting of: Fenollaria, Anaeroglobus, Anaerococcus, Coprococcus, Prevotella, Varibaculum, Corynebacterium, Thalassobacillus, Staphylococcus, Priestia, Butyricimonas, Finegoldia, Mobiluncus, Cutibacterium, Peptoniphilus, Veillonella, and Gardnerella. In some embodiments, the panel of bacterial taxa comprises at least one taxon selected from the group consisting of: Staphylococcus aureus, Fenollaria massiliensis, Priestia megaterium, Coprococcus catus, Butyricimonas faecihominis, Anaeroglobus geminatus, Anaerococcus octavius, Prevotella corporis, Varibaculum anthropi, Corynebacterium urealyticum, Thalassobacillus hwangdonensis, Corynebacterium tuberculostearicum, Staphylococcus intermedius, Finegoldia magna, Mobiluncus curtisii, Cutibacterium namnetense, Peptoniphilus harei, Priestia aryabhattai, Veillonella atypica, Prevotella timonensis, Prevotella bivia, and Gardnerella vaginalis. The method allows for the analysis of subsets of these taxa, or the entire panel, wherein each taxon can be identified by the V4 region of a 16S rRNA gene sequence having at least 97% identity to specific reference sequences (e.g., SEQ ID NOs: 3-24).

In embodiments where the sample is obtained during the secretory phase, the panel comprises at least one taxon selected from the group consisting of: Ureaplasma, Niallia, Murdochiella, Gardnerella, Lactobacillus, Lawsonella, Corynebacterium, Priestia, Finegoldia, and Dialister. In some embodiments, the panel of bacterial taxa comprises at least one taxon selected from the group consisting of Ureaplasma urealyticum, Niallia oryzisoli, Murdochiella asaccharolytica, Gardnerella vaginalis, Lactobacillus iners, Lactobacillus jensenii, Lawsonella clevelandensis, Corynebacterium kroppenstedtii, Priestia megaterium, Lactobacillus crispatus, Finegoldia magna, Dialister hominis, Lactobacillus vaginalis, and Ureaplasma parvum. In some embodiments, the taxa of the panel can be defined by their corresponding 16S rRNA V4 gene sequences (e.g., SEQ ID NOs:5, 16, and 24-35).

To ensure the appropriate panel is applied, the method can further comprise measuring a serum progesterone level. A level below a reference threshold (e.g., 1.08 ng/mL) confirms the proliferative phase, while a level above said threshold confirms the secretory phase.

The assessment further comprises the calculated Functional Dysbiosis Score (FDS). In some embodiments, the FDS is calculated using the formula: FDS=0.5×(1−A_{Lacto})+10×A_{Patho}, wherein A_{Lacto} is the relative abundance of Lactobacillus and A_{Patho} is the cumulative relative abundance of a plurality of pathogenic taxa. These pathogenic taxa can include genera such as Gardnerella, Prevotella, Anaerococcus, Streptococcus, Megasphaera, Mobiluncus, Sneathia, Atopobium, Peptoniphilus, Mycoplasmoides, Ureaplasma, Bacteroides, Peptostreptococcus, and Dialister.

The integration of these biological variables is handled by a trained machine learning classifier, such as a Random Forest classifier. This classifier can be trained on datasets of confirmed endometriosis cases and controls using cross-validation (e.g., repeated random subsampling). The features for the classifier can be selected via multivariable association analysis (e.g., MaAsLin2) that controls for confounding variables such as age and Body Mass Index (BMI), ensuring the diagnostic output is specific to the disease pathology.

The methods utilize advanced sequencing techniques. Obtaining the dataset typically comprises extracting genomic DNA from the sample, amplifying a variable region of the bacterial 16S rRNA gene (preferably the V4 region using specific primers, such as SEQ ID NOs: 1 and 2), and sequencing the amplicons. To enhance specificity, the method can involve bioinformatically removing sequencing reads that map to a human reference genome.

The methods are applicable to abroad range of biological samples, with a particular focus on the female reproductive tract. Suitable samples include uterine tissue (e.g., endometrial biopsies), uterine fluid, vaginal mucus, cervicovaginal fluid, and endometrial cells. The subject assessed can be symptomatic (presenting with clinical indicators such as dysmenorrhea, chronic pelvic pain, or infertility) or asymptomatic. In some embodiments, the microbiome assessment is corroborated by measuring additional protein or miRNA biomarkers.

In some embodiments, the present disclosure provides a method of treatment comprising administering a therapy for endometriosis to a subject identified as having a high likelihood of the disease based on the microbiome characterization. Suitable treatments include pain medication, hormone therapies (e.g., GnRH agonists/antagonists, oral contraceptives, progestins), or surgical procedures such as laparoscopic excision.

In some embodiments, the disclosure provides kits for assessing endometriosis. These kits comprise the physical means for obtaining the dataset (e.g., 16S rRNA V4-specific primers and sample collection containers) and a non-transitory computer-readable medium. The medium stores executable instructions that cause a processor to receive the sequencing data, quantify the appropriate phase-specific bacterial taxa, calculate the FDS, and input these features into the stored machine learning classifier to generate the diagnostic output.

### 5. BRIEF DESCRIPTION OF DRAWINGS

FIG. 1 provides an overview of the study design described in Example 3. A total of 266 samples were analyzed, including 138 from participants in the proliferative phase (78 patients and 60 controls) and 128 from the secretory phase (88 patients and 40 controls). Targeted sequencing of the 16S rRNA V4 hypervariable region was performed. Bacterial reads were annotated using the Greengenes2 database. Differential and informative taxa were identified for machine learning-based prediction of endometriosis.

FIGS. 2A-2B provides the analyses for both alpha and beta diversity. FIG. 2A provides the alpha diversity comparisons between patients and controls within each menstrual phase.

FIG. 2B provides the beta diversity comparisons between patients and controls in both proliferative and secretory phases.

FIGS. 3A-3B provides genus-level relative abundance and microbial community profiles in proliferative phase samples (FIG. 3A) and in secretory phase samples (FIG. 3B).

FIGS. 4A-4C provide taxa-level analysis. FIG. 4A shows the regression coefficients of differential taxa identified by MaAsLin2 (p≤0.05) that distinguish endometriosis from controls in proliferative and secretory phase samples. FIG. 4B provides boxplots illustrating the relative abundance distribution of differential taxa in proliferative and secretory phases. FIG. 4C shows bacterial taxa identified as informative features through machine learning-based selection using the Random Forest algorithm.

FIGS. 5A-5B show the predictive performance of the differential microbial profile from the proliferative phase (FIG. 5A) and the secretory phase (FIG. 5B) in classifying endometriosis.

### 6. DETAILED DESCRIPTION

Endometriosis, a chronic inflammatory disease affecting 10-15% of reproductive-age women globally, involves the growth of endometrial-like tissue outside the uterus, causing pain, infertility, and reduced quality of life. Diagnosis is often delayed due to the need for laparoscopic examination. Current diagnostic methods, including imaging and clinical evaluation, have limitations in sensitivity and specificity, highlighting a need for reliable screening and diagnostic tools.

Dysbiosis, or imbalance in the microbiome, has been implicated in various diseases. Provided herein are novel methods for assessing risk for endometriosis by analyzing the microbiome, offering a novel and effective diagnostic tool for this prevalent condition.

Before the present disclosure is further described, it is to be understood that the disclosure is not limited to the particular embodiments set forth herein, and it is also to be understood that the terminology used herein is for the purpose of describing particular embodiments, and is not intended to be limiting.

### 6.1 DEFINITIONS

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

As used herein in the specification, “a” or “an” may mean one or more. As used herein in the claim(s), when used in conjunction with the word “comprising,” the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein “another” or “additional” may mean at least a second or more.

As used herein, the term “about” is used to indicate that a value includes the inherent variation of error for the device, the method being employed to determine the value, or the variation that exists among the study subjects. The term “about” encompasses the exact number recited. In some embodiments, “about” means within plus or minus 10% of a given value or range. In certain embodiments, “about” means that the variation is ±5%, ±4%, ±3%, ±2%, ±1%, ±0.5%, ±0.2%, or 0.1% of the value to which “about” refers. In some embodiments, “about” means that the variation is +1%, +0.5%, ±0.2%, or +0.1% of the value to which “about” refers.

The terms “nucleic acid,” “polynucleotide,” and their grammatical equivalents, are used interchangeably herein and refer to a polymer or oligomer of nucleotides of any length. The nucleotides can be deoxyribonucleotides, ribonucleotides, modified nucleotides or bases (such as methylated, hydroxymethylated, or glycosylated), non-natural nucleotides, non-nucleotide building blocks that exhibit similar structure and/or function as natural nucleotides (i.e., “nucleotide analogs”), and/or any substrate that can be incorporated into a polymer by DNA or RNA polymerase. The nucleic acids or polynucleotides can be heterogenous or homogenous in composition, can be isolated from naturally occurring sources, or can be artificially or synthetically produced. In addition, the nucleic acids or polynucleotides can be DNA (e.g., cDNA or genomic DNA) or RNA (e.g., mRNA, anti-sense RNA, siRNA, and miRNA), or a mixture thereof, and can exist permanently or transitionally in single-stranded or double-stranded form, including homoduplex, heteroduplex, and hybrid states.

Conventional notation is used herein to describe nucleotide sequences: the left-hand end of a single-stranded nucleotide acid is the 5′-end; the left-hand direction of a double-stranded nucleic acid is referred to as the 5′-direction; the right-hand end of a single-stranded nucleotide acid is the 3′-end; the right-hand direction of a double-stranded nucleic acid is referred to as the 3′-direction. The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

The terms “polypeptide,” “peptide,” “protein,” and their grammatical equivalents as used interchangeably herein refer to polymers of amino acids of any length, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification.

The terms “microbiome” and “microbiota” as used interchangeably herein refer to the totality of microbial life forms within a given habitat or host. Examples of microbiomes include the intestinal, fecal, oral, nasal, vaginal, skin, and lung microbiomes, as well as those found in waste treatment systems, soil, plants, or used in food fermentation processes. The term “endometrial microbiome” specifically refers to the unique community of microorganisms residing within the endometrial environment, which can influence reproductive health and disease states.

The term “microbiome feature” as used herein in connection with a disease or condition (e.g., endometriosis) refer to a characteristic of the microbiome that can indicate the presence of the disease or condition. A microbiome feature can be the presence of specific microbial biomarkers in the vaginal or endometrial microbiomes. A microbiome feature can also be altered levels or abundance of certain bacteria that correlate with the disease.

As used herein, the term “biomarker” refers to a measurable indicator in the body of a subject that can reflect a biological process, condition, or a response to a therapeutic intervention.

For example, a biomarker for endometriosis means a measurable indicator in the body of a subject that can signal the presence, absence, or stage of the disease, or the risk of developing the disease. Biomarkers encompass a variety of biological entities, including, for example, proteins, nucleic acids (e.g., mRNA), metabolites, and also whole cells or microorganisms (e.g., bacteria in the microbiome). Biomarkers can be used for diagnostic, prognostic, predictive, or monitoring purposes in health and disease management, aiding in early detection, disease progression assessment, and evaluating the effectiveness of treatments.

Microbiome-based biomarkers, refer to specific bacterial species in the microbiome that can serve as biomarkers. For example, microbial biomarkers for endometriosis refer to the bacteria that are present in the gut and/or vaginal microbiomes of women. The presence, absence, or abundance of these bacteria can serve as indicators for the presence of endometriosis, the risk of developing endometriosis, or the likelihood of progression into a later stage of the disease. For example, the presence of a bacterial species of Gardnerella, or Streptococcus in endometrial tissue can serve as biomarkers for endometriosis.

As used herein, the term “signature” refers to a distinctive pattern, expression profile, or presence/absence of biomarkers that serves as an identifier of a specific biological state or condition. A “protein signature” refers to the presence, absence, or specific expression levels of a set of one or more protein biomarkers. A “miRNA signature” refers to the presence, absence, or specific expression levels of a set of one or more miRNAs). In the context of this disclosure, a “microbial signature” or “microbiome signature” refers to the specific combination of bacterial taxa relative abundances and/or derived scores (like FDS) that distinguishes a subject with endometriosis from a healthy control.

As used herein, the term “taxon” (plural “taxa”) refers to a group of one or more populations of an organism or organisms seen by taxonomists to form a unit. A taxon is usually given a name and a rank, although neither is a requirement. In the context of the present disclosure, a “bacterial taxon” refers to a grouping of bacteria at any level of the taxonomic hierarchy, including but not limited to phylum, class, order, family, genus, species, or strain (e.g., an Operational Taxonomic Unit (OTU) or Amplicon Sequence Variant (ASV)). A “pathogenic taxon” refers to a bacterial taxon that is associated with a disease state, dysbiosis, or inflammation. In some embodiments of the provided method, “pathogenic taxa” refer to those genera or species whose increased abundance is negatively correlated with a healthy uterine or vaginal environment (e.g., Gardnerella, Prevotella), and which contribute to the calculation of the Functional Dysbiosis Score (FDS).

As used herein consistently with its understanding in the art, the term “16S rRNA” refers to the 16S ribosomal RNA, to a bacterial gene encoding a component of the 30S small subunit of a prokaryotic ribosome, which binds to the Shine-Dalgamo sequence. The term “16S rRNA gene” refers to the DNA sequence in the bacterial genome that codes for the 16S ribosomal RNA molecule. The gene contains both conserved regions (useful for universal amplification) and hypervariable regions (useful for identification). The “V4 region” of bacterial 16S rRNA gene refers to the fourth hypervariable region of the 16S rRNA gene, a specific segment of DNA often used for taxonomic classification due to its high variability among different bacterial species.

As used herein, the term “assess” refers to the process of evaluating the status, condition, or future trajectory of a subject. In the specific context of “assessing a likelihood of endometriosis,” the term refers to generating a quantitative or qualitative output—such as a probability score, risk index, or classification label—that indicates the statistical probability that a subject currently harbors endometrial lesions or is at risk of developing them. Importantly, “assess” in this context provides a risk stratification to guide clinical decision-making and does not necessarily require a definitive pathological confirmation (e.g., via laparoscopic surgery). The term encompass diagnosing the current presence of endometriosis, predicting the risk of future onset, prognosticating disease progression (e.g., advancement to a later stage), and monitoring a subject's response to therapeutic intervention. In embodiments involving prediction of future development or progression, the assessment can cover a predictive window of between about 6 months and 2 years, or more specifically, between about 6 months and 12 months.

As used herein, the term “measure” or its grammatical equivalent, refers to the process of conducting a qualitative, a semi-quantitative or a quantitative means for, e.g., detecting and determining the level or abundance of a biomarker, using technology available to the skilled artisan. Measurement can be relative or absolute. Measuring the expression of a biomarker can include, e.g., determining whether the expression product of the biomarker is present or absent, or the amount or abundance of the biomarker.

The term “identity” or “sequence identity” as used herein refers to the degree of similarity between two nucleotide or protein sequences, expressed as a percentage of matches (identical residues) in an alignment. Sequence identity is determined by comparing sequences to maximize overlap and minimize gaps, using either global or local alignment algorithms, depending on the length and similarity of the sequences. Commonly used algorithms include the Needleman-Wunsch algorithm for global alignment of sequences of similar lengths and the Smith-Waterman algorithm for local alignment of sequences with substantial length differences. Other methods include the search for similarity method by Pearson & Lipman, the BLAST algorithm (e.g., WU-BLAST-2, gapped BLAST), and tools like GAP, BESTFIT, FASTA, and TFASTA in the Wisconsin Genetics Software Package. Online resources such as BLAST (http://blast.ncbi.nlm.nih.gov) and EMBOSS Needle (http://www.ebi.ac.uk/Tools/emboss/) can be employed for determining sequence identity. The parameters for these algorithms can be adjusted to optimize alignment sensitivity, but unless otherwise specified, identity is determined using default settings, such as the BLOSUM62 scoring matrix, with specific gap penalties. The percent identity indicates how closely two sequences match over their full length, providing insight into their similarity or evolutionary relationship.

As used herein, terms “complementary” and “complementarity” refer to the relationship between two nucleic acid molecules having the capacity to form hydrogen bond(s) with one another by either traditional Watson-Crick base-paring or other non-traditional types of pairing. The two DNA/RNA strands with complementary sequences bind to form a duplex that follows the Watson-Crick base-pairing rules: A binds to T (U) with two hydrogen bonds; G binds to C with three hydrogen bonds. The degree of complementarity between two nucleotide sequences can be indicated by the percentage of nucleotides in a nucleotide sequence which can form hydrogen bonds (e.g., Watson-Crick base pairing) with a second nucleotide sequence (e.g., about 50%, about 60%, about 70%, about 80%, about 90%, and 100% complementary). Two nucleotide sequences are “perfectly complementary” or “100% complementary” if all the contiguous nucleotides of a nucleotide sequence will hydrogen bond with the same number of contiguous nucleotides in a second nucleotide sequence. Two nucleotide sequences are “substantially complementary” if the degree of complementarity between the two nucleotide sequences is at least 60% (e.g., at least 65%, at least 70%, at least 75%, at least 80%, at least 85%, at least 90%, at least 95%, at least 97%, at least 98%, at least 99%, or 100%) over a region of at least 8 nucleotides (e.g., at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, or more nucleotides), or if the two nucleotide sequences hybridize under at least moderate, or, in some embodiments high, stringency conditions. Exemplary stringency conditions are descried in, e.g., Sambrook, J., Molecular Cloning: A Laboratory Manual, Cold Spring Harbor Laboratory Press; 4th edition (Jun. 15, 2012), and Ausubel et al., eds., SHORT PROTOCOLS IN MOLECULAR BIOLOGY, 5th ed., John Wiley & Sons, Inc., Hoboken, N.J. (2002).

The term “hybridize” and its grammatically equivalents when used in connection with nucleotide sequences refer to the association formed between and/or among sequences having complementarity. The term “specifically hybridize”, as used herein, refers to the conditions which allow the hybridization of two polynucleotides under high stringent conditions or moderately stringent conditions. The “stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and the target sequence, the higher the relative temperature which must be used. As a result, it follows that higher relative temperatures would tend to make the reaction conditions more stringent, while lower temperatures less so.

As used herein, the term “hybridizing conditions” is intended to mean those conditions of time, temperature, and pH, and the necessary amounts and concentrations of reactants and reagents, sufficient to allow at least a portion of complementary sequences to anneal with each other. As it is well known in the art, the time, temperature, and pH conditions required to accomplish hybridization depend on the size of the oligonucleotide probe or primer to be hybridized, the degree of complementarity between the oligonucleotide probe or primer and the target, the nucleotide type (e.g., RNA, or DNA) of the oligonucleotide probe or primer and the target, and the presence of other materials in the hybridization reaction mixture. The actual conditions necessary for each hybridization step are well known in the art or can be determined without undue experimentation. General parameters for specific (i.e., stringent) hybridization conditions for nucleic acids are described in Sambrook, et al., MOLECULAR CLONING: A LABORATORY MANUAL (3RD EDITIoN, 2001). One of skills in the art will in particular appreciate that as the oligonucleotides become shorter, it may become necessary to adjust their length to achieve a relatively uniform melting temperature for satisfactory hybridization results.

The terms “low stringency,” “medium stringency,” “medium/high stringency,” “high stringency” and “very high stringency” refer to conditions of hybridization. Suitable experimental conditions for determining hybridization between a nucleotide probe and a homologous DNA or RNA sequence involves presoaking of the filter containing the DNA fragments or RNA to hybridize in 5×SSC (Sodium chloride/Sodium citrate for 10 min, and prehybridization of the filter in a solution of 5×SSC, 5×Denhardt's solution, 0.5% SDS and 100 μg/ml of denatured sonicated salmon sperm DNA, followed by hybridization in the same solution containing a concentration of 10 ng/ml of a random-primed 32P-dCTP-labeled (specific activity >1×109 cpm/pg) probe for 12 hours at ca. 45° C. (Feinberg and Vogelstein, 1983). For example, to achieve various stringency conditions the filter can be washed twice for 30 minutes in 2×SSC, 0.5% SDS and at least 55° C. (low stringency), more preferably at least 60° C. (medium stringency), still more preferably at least 65° C. (medium/high stringency), even more preferably at least 70° C. (high stringency), and even more preferably at least 75° C. (very high stringency).

The term “oligonucleotide,” as used herein, refers to a single-stranded DNA or RNA molecule, preferably with up to 35, 30, 25, 20, 19, 18, 17, 16, 15, 14 or 13 bases in length (upper limit). The oligonucleotides can be DNA or RNA molecules, preferably of at least 2, at least 5, at least 10, at least 12, at least 15, at least 16, at least 17, at least 18, at least 19, at least 20, at least 21, at least 22, at least 23, at least 25 nucleotide bases in length (lower limit). Ranges of base lengths can be combined in all different manners using the afore-mentioned lower and upper limits, for example at least 2 and up to 30 bases, at least 10 and up to 15 bases, at least 5 and up 15 bases or at least 15 and up to 18 bases.

The term “primer set,” as used herein, refers to a set of oligonucleotides of RNA or DNA (preferably about 15-35 bases) that specifically hybridize to target regions of a nucleic acid sequence and serve as starting points for DNA synthesis. They are required for DNA amplification mediated by a DNA polymerase in reactions such as the PCR technique. The relative amount, concentration, and/or average size of each amplicon can then be analyzed using various techniques known to those skilled in the art, including gel electrophoresis or methods based on RT-PCR. Additionally, these primers can be used for sequencing the target nucleic acid, followed by further steps familiar to person of ordinary skill in the art. In some embodiments, a primer set can specifically hybridize to the hypervariable regions of the 16S rRNA gene.

The term “probe,” as used herein, refers to DNA or RNA oligonucleotide sequences that hybridize by complementarity with a specific sequence. In other words, the probe hybridizes to specific single-stranded nucleic acid (DNA or RNA) whose base sequence allows probe-target base pairing due to complementarity between the probe and the target. In a preferred aspect, the subsequent hybrid can be detected using techniques known by the expert in the field. For instance, the probe can be labelled with a marker that can be radioactive or (a) fluorescent molecule(s) and immobilized on a membrane or in situ. Commonly used markers are ³²P (a radioactive isotope of phosphorus incorporated into the phosphodiester bond in the probe DNA) or Digoxigenin, which is a non-radioactive, antibody-based marker. DNA sequences or RNA transcripts that have moderate to high sequence similarity to the probe are then detected by visualizing the hybridized probe via autoradiography or other imaging techniques. Normally, either X-ray pictures are taken of the filter, or the filter is placed under UV light, or under a microscope for the detection of the fluorescently labelled probe. Detection of sequences with moderate or high similarity depends on how stringent the hybridization conditions were applied—high stringency, such as high hybridization temperature and low salt in hybridization buffers, permits only hybridization between nucleic acid sequences that are highly similar, whereas low stringency, such as lower temperature and high salt, allows hybridization when the sequences are less similar.

As used herein, the term “amplify” or its grammatical equivalent refers to the production of multiple copies of a specific nucleic acid sequence, typically using Polymerase Chain Reaction (PCR). An “amplicon” is the product of that amplification event—a piece of DNA or RNA that is the source and/or product of amplification or replication events. In the context of the present disclosures, amplicons are typically copies of the V4 region of the 16S rRNA gene.

As used herein, the term “sequencing” refers to determining the order of nucleotides (base sequences) in a nucleic acid molecule. The term encompasses all methods of determining the nucleotide sequence of a nucleic acid, including identifying specific nucleotides (A, T, C, G) or their analogs. In some embodiments, the sequencing can be “Next Generation Sequencing” (NGS) or “high-throughput sequencing,” which describes technologies that allow for the parallel sequencing of a large number (e.g., millions) of DNA fragments simultaneously. Examples of NGS platforms include, but are not limited to, sequencing-by-synthesis platforms (e.g., Illumina MiSeq, HiSeq, NovaSeq), ion semiconductor sequencing (e.g., Ion Torrent), pyrosequencing (e.g., 454), and single-molecule real-time sequencing (e.g., Pacific Biosciences, Oxford Nanopore). As used herein, the term “deep sequencing” refers to sequencing a target nucleic acid region (such as the 16S rRNA gene) at a high depth of coverage, meaning that the region is sequenced a large number of times (e.g., thousands or millions of reads per sample). Deep sequencing allows for the detection of low-abundance sequences, rare variants, or minority microbial populations that would be missed by traditional sequencing methods. In the context of the microbiome, deep sequencing is utilized to accurately profile the low-biomass environment and identify specific bacterial taxa present at low relative abundances.

As used herein, the term “dataset” refers to a collection of data. In the context of bioinformatics, a “dataset representing a plurality of nucleic acid sequences” refers to the digital information generated from a sequencing run, comprising the sequence reads (strings of A, T, C, G nucleotides) derived from the sample. This dataset serves as the raw or processed input for downstream quantification of bacterial taxa.

As used herein, the term “sequencing read” refer to the inferred sequence of base pairs (or base pair probabilities) corresponding to all or part of a single DNA fragment, generated by sequencing. The term “map” or “mapping” refers to the bioinformatic process of aligning these short sequencing reads to a reference sequence to determine their most likely point of origin based on sequence similarity. The term “human reference genome” refers to a standardized digital representation of the human genetic sequence (e.g., the GRCh38/hg38 assembly) used as a coordinate system for aligning reads. Accordingly, “sequencing reads mapping to a human reference genome” refer to those reads that align with high similarity/identity to human DNA sequences rather than microbial sequences. “Bioinformatically removing” means using computational tools to filter out these host-derived reads from the dataset so they are not counted as microbial data, thereby reducing noise.

As used herein, the term “level” of a biomarker refers to the amount or abundance of a biomarker (e.g., a bacterial species). As used herein, the term “reference level” refers to a predetermined level of a biomarker that can be used to determine the significance of the level of the biomarker in a sample from a subject. A reference level of a biomarker can be the average level of the biomarker in samples from a healthy population. A reference level of a biomarker can also be a cut-off value determined by a person of ordinary skill in the art through statistical analysis of the levels of the biomarker in a sample population and the of the clinical outcome of the individuals in the sample population. For example, by analyzing the levels of certain bacterial species in the endometrial microbiomes of individuals of a sample population and the clinical outcome of these individuals with respect to endometriosis, a person of ordinary skill in the art can determine a cut-off value as the reference level of the bacterial species, wherein a subject is likely to have, develop, or progress into an advanced stage of endometriosis if the level of the bacterial species in the endometrial microbiome of the subject is different from the reference level.

As used herein and understood in the art, the terms “lowered,” “decreased,” or “down-regulated” when used in connection to the level of a biomarker (e.g., a bacterial species) means that the level of the marker in the sample is less than the reference level. For example, a decreased level of a bacterial species detected in a sample of a subject means that the level of the bacterial species in the sample is lower compared to a reference level. In some embodiments, the level of the biomarker can be at least 10%, at least 20%, at least 30%, at least 40%, at least 50%, at least 60%, at least 70%, at least 80%, at least 90% less than the reference level.

As used herein and understood in the art, the terms “elevated,” “increased,” or “up-regulated,” when used in connection to the level of a biomarker (e.g., a bacterial species) means that such level in the sample is higher than the reference level. For example, an increased level of a bacterial species detected in a sample of a subject means that the level of the bacterial species in the sample is higher compared to a reference level. In some embodiments, the level of the biomarker can be at least 10%, at least 20%, at least 30%, at least 40%, at least 50%, at least 60%, at least 70%, at least 80%, at least 90%, at least 2.0 fold, at least 3.0 fold, at least 4.0 fold, at least 5.0 fold, at least 6.0 fold, at least 7.0 fold, at least 8.0 fold, at least 9.0 fold, or at least 10.0 fold than the reference level.

Comparing the level of a biomarker usually means comparison of corresponding parameters or values, e.g., an absolute amount is compared to an absolute reference amount, or an intensity signal obtained from the biomarker in a sample is compared to the same type of intensity signal obtained from a reference sample. The comparison can be carried out manually or assisted by computer. In some embodiments, the comparison is carried out by a computing device. The value of the measured or detected level of the biomarker in a sample from a subject and the reference level can be, e.g., compared to each other and the said comparison can be automatically carried out by a computer program executing an algorithm for the comparison. The computer program carrying out the said evaluation can provide the desired assessment in a suitable output format. For a computer-assisted comparison, the value of the measured amount can be compared to values corresponding to suitable references which are stored in a database by a computer program. The computer program can further evaluate the result of the comparison, i.e., automatically provide the desired assessment in a suitable output format.

The term “abundance” as used herein refers to the quantity or prevalence of a bacterial taxon in a sample. The term “relative abundance” refers to the proportion of a specific bacterial taxon relative to the total microbial composition analyzed in the sample. It is typically expressed as a percentage or a fraction (0 to 1). Relative abundance is calculated by dividing the quantified value of a specific taxon (e.g., the number of sequencing reads mapping to that taxon) by the total number of valid sequencing reads (or total library size) for that sample. As used herein, the term “cumulative relative abundance” refers to the sum of the relative abundances of a defined subset of taxa within a sample. For example, calculating the cumulative relative abundance of pathogenic taxa involves summing the individual relative abundance percentages of all bacterial genera identified as pathogenic in the panel.

The term “Functional Dysbiosis Score” or “FDS” refers to a composite metric calculated to quantify the degree of microbial imbalance in a sample. In some embodiments described herein, the FDS is calculated using a formula that weighs the presence of protective bacteria (e.g., Lactobacillus) against the cumulative presence of pathogenic bacteria. For example, the FDS can be calculated by the formula: FDS=0.5×(1−A_{Lacto})+10×Aₚₐₜₕₒ, wherein A_{Lacto} is the relative abundance of Lactobacillus and Aₚₐₜₕₒ is the cumulative relative abundance of the plurality of pathogenic taxa.

As used herein, the term “classifier” refers to an algorithm or mathematical function that maps input data (features) to a category or continuous output. A “trained machine learning classifier” refers to a classifier whose internal parameters (e.g., weights, decision nodes) have been learned from a training dataset of labeled examples (e.g., samples known to be from endometriosis cases vs. controls). A “Random Forest classifier” is a specific type of ensemble learning method that operates by constructing a multitude of decision trees at training time and outputting the class that is the mode of the classes (classification) or mean prediction (regression) of the individual trees. As used herein, “output” or “classification output” refers to the result generated by the machine learning classifier after processing the input features. This output can take the form of a binary label (e.g., “Positive,” “Negative”), a probability score (e.g., “0.85 probability of disease”), or a continuous variable indicating risk level.

As used herein, the term “multivariable association analysis” refers to a statistical technique used to analyze data that arises from more than one variable. It determines the contribution of multiple independent variables (e.g., bacterial abundance, age, BMI) to a dependent variable (e.g., disease status). “Microbiome Multivariable Associations with Linear Models” or “MaAsLin2” refers to a specific comprehensive R package and statistical framework for determining multivariable associations between clinical metadata and microbial omics features, capable of handling sparse, high-dimensional data and controlling for covariates. As used herein, a “confounding variable” (or confounder) is a variable that influences both the dependent variable and independent variable, causing a spurious association. In some embodiments, in the context of microbiome studies, age and Body Mass Index or BMI are considered confounders. To be “controlled” for a confounding variable means that the statistical analysis includes the confounder as a covariate, mathematically isolating the effect of the microbiome on the disease independent of the confounder.

As used herein, the term “subject” as used herein refers to any animal (e.g., a mammal), including, but not limited to, humans, non-human primates, canines, felines, rodents, and the like. The subject can be a human. The subject can be a human female. The subject can be a healthy subject. A subject can have a particular disease or condition. The subject can have at least one symptom associated with endometriosis, such as pelvic pain, dysmenorrhea, or infertility. In some embodiments, the subject is a young or adolescent human female. In some embodiments, the subject is a human female aged between 12-60 years. In some embodiments, the subject is a human female aged about 20 years old, 30 years old, 40 years old, 50 years old or 60 years old.

As used herein, the term “subject” refers to any animal (e.g., a mammal), including, but not limited to, humans, non-human primates, canines, felines, rodents, and the like. In some embodiments, the subject is a human female, including adolescents and adults (e.g., aged 12-60 years). A subject can present with a “clinical indicator of endometriosis,” which refers to a symptom, sign, or patient-reported outcome traditionally associated with the disease. These indicators include, but are not limited to, dysmenorrhea (painful menstruation), deep dyspareunia (pain during intercourse), chronic pelvic pain, dyschezia (painful defecation), dysuria (painful urination), fatigue, and infertility. The subject can also be “asymptomatic,” meaning the subject does not exhibit overt physical symptoms of endometriosis (such as pelvic pain) at the time of assessment, even though they may harbor ectopic endometrial lesions or present with infertility as the sole indication.

As used herein, the term “sample,” refers to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. In the context of the present disclosure, the sample can comprise material derived from the female reproductive tract.

Examples of samples include, but are not limited to: fluid samples such as cervicovaginal fluid, vaginal mucus, cervical mucus, uterine lavage fluid, uterine fluid, menstrual effluent, interstitial fluid, cervical secretion, semen, and blood or serum; and solid or cellular samples such as endometrial tissue (e.g., obtained via Pipelle biopsy or curettage), uterine tissue, vaginal mucosa (e.g., obtained via swab scrubbing), reproductive cells, cervical cells, endometrial cells, fallopian cells, ovarian cells, and the natural flora found in a female reproductive tract. A sample can be obtained in vivo or in situ and provides the source material for extracting the genomic DNA used in the sequencing assays described herein.

As used herein, the term “treat” or its grammatical equivalent refers to executing a protocol or plan, which can include administering one or more drugs or active agents to a patient to alleviate signs or symptoms of the disease or the recurrence of the disease. Treatment can also include medical procedures such as surgeries. Desirable effects of treatment include decreasing the rate of disease progression, ameliorating or palliating the disease state, and remission, increased survival, improved quality of life or improved prognosis. Alleviation or prevention can occur prior to signs or symptoms of the disease or condition appearing, as well as after their appearance. As used herein, a “treatment” does not require complete alleviation of signs or symptoms and does not require a cure. As used herein, the term “therapeutic beneficial” or “therapeutically effective” when used in connection with a treatment refers to the property of the treatment that promotes or enhances the well-being of the subject. This includes, but is not limited to, a reduction in the frequency, severity, or rate of progression of the signs or symptoms of a disease. For example, treatment of endometriosis may result in, for example, a reduction in pain, or pregnancy.

As used herein, the term “administer” or its grammatical equivalent refers to the act of delivering, or causing to be delivered, a compound or a pharmaceutical composition to the body of a subject by a method described herein or otherwise known in the art, and the act of providing a medical procedure on the subject for the purpose of treating the subject. Administering a compound or a pharmaceutical composition includes prescribing a compound or a pharmaceutical composition to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

Nomenclature for nucleotides, nucleic acids, nucleosides, and amino acids used herein is consistent with International Union of Pure and Applied Chemistry (IUPAC) standards (see, e.g., bioinformatics.org/smsylupac.html). Exemplary genes and polypeptides are described herein with reference to GenBank numbers, GI numbers and/or SEQ ID NOS. It is understood that one skilled in the art can readily identify homologous sequences by reference to sequence sources, including but not limited to Uniprot (https://www.uniprot.org/), GenBank (ncbi.nlm.nih.gov/genbank/) and EMBL (embl.org/).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range or the characteristics being described.

### 6.2 METHODS FOR ASSESSING LIKELIHOOD OF ENDOMETRIOSIS

Individuals with endometriosis exhibit increases or decreases in one or more taxonomic groups (e.g., bacterial species, genera) or functional groups within the vaginal, endometrial or other relevant microbiomes compared to healthy subjects. The present disclosure provides robust, non-invasive, and highly specific methods for characterizing the uterine and/or vaginal microbiome to assess a likelihood of endometriosis in a subject. The methods disclosed herein represent a significant departure from conventional diagnostic reliance on laparoscopic surgery. By integrating high-throughput sequencing of bacterial 16S rRNA genes with advanced machine learning algorithms, methods disclosed in the present disclosures overcome the historical challenges of low microbial biomass and host DNA contamination in uterine samples.

### 6.2.1 General Methods

In some embodiments, a method is provided for assessing the likelihood of a subject having endometriosis or developing endometriosis by analyzing changes in the microbiome. In some embodiments, provided herein are methods for assessing the likelihood of a subject having endometriosis comprising analyzing microorganism nucleic acids in a sample from the subject to determine the presence or absence of a microbiome feature associated with endometriosis, wherein the presence of the microbiome feature indicates that the subject has an increased likelihood of having endometriosis.

In some embodiments, the methods provided herein comprise obtaining a biological sample (e.g., uterine tissue, fluid, or vaginal swab), extracting genomic DNA, amplifying a target marker (e.g., the V4 region of the 16S rRNA gene), and quantifying the relative abundance of a specific panel of bacteria. This abundance data is then optionally combined with a calculated Functional Dysbiosis Score (FDS) and processed by a trained machine learning classifier (e.g., a Random Forest model) to generate a probability score or classification output indicating the presence or absence of endometriosis. In some embodiments, provided herein are methods for characterizing a microbiome to assess a likelihood of endometriosis in a subject, comprising: (a) obtaining a dataset representing a plurality of nucleic acid sequences derived from a sample obtained from the subject; (b) quantifying, from the dataset, a relative abundance of a panel of bacterial taxa; (c) calculating a Functional Dysbiosis Score (FDS) for the sample based on a relative abundance of Lactobacillus spp. and a cumulative relative abundance of a plurality of pathogenic taxa; and (d) processing the relative abundance of the panel of bacterial taxa and the FDS using a trained machine learning classifier to generate a classification output indicating the presence or absence of endometriosis.

Crucially, the present disclosure identifies that the microbial signature associated with endometriosis is phase-dependent. Accordingly, in some embodiments, the methods described herein characterize the microbiome specifically during the proliferative phase or the secretory phase of the menstrual cycle. It has been discovered that the diagnostic accuracy is significantly enhanced when the specific panel of bacterial taxa is matched to the subject's menstrual phase.

In some embodiments, methods provided herein comprise analyzing microorganism nucleic acids in a sample from the subject to determine a microbiome feature associated with endometriosis, wherein the presence of the microbiome feature indicates that the subject has endometriosis. In some embodiments, the sample is obtained during the secretory phase of a menstrual cycle. In some embodiments, the sample is obtained during the proliferative phase of a menstrual cycle. The analysis of microbiome features from samples obtained from both the secretory and proliferative can help assess the risk for endometriosis and determine the disease stage (early or late).

### 6.2.2 Determination of Menstrual Cycle Phase

Because the microbial signatures associated with endometriosis are distinct depending on the hormonal milieu of the uterus, determining the menstrual cycle phase of the subject ensures diagnostic accuracy. In some embodiments, the phase of the menstrual cycle is determined prior to, or concomitant with, the collection of the biological sample. The menstrual cycle is generally divided into the proliferative phase (follicular phase) and the secretory phase (luteal phase), separated by ovulation. The methods described herein can utilize any reliable clinical, biochemical, or histological means known in the art to categorize the subject's status into one of these two phases.

In some embodiments, the menstrual phase is determined biochemically by measuring the concentration of serum progesterone in the subject. Progesterone levels are low during the proliferative phase and rise significantly following ovulation during the secretory phase. Accordingly, the method can comprise measuring a serum progesterone level and comparing it to a reference level.

In some embodiments, a serum progesterone level below the threshold indicates the subject is in the proliferative phase, whereas a level above the threshold indicates the subject is in the secretory phase. In one exemplary embodiment, the reference level is about 1.08 ng/mL. However, the methods disclosed herein are not limited to this precise value; depending on the assay sensitivity and calibration, the reference level may be set at about 1.0 ng/mL, 1.5 ng/mL, 2.0 ng/mL, or 3.0 ng/mL. Additional hormonal markers can also be quantified to refine the phase determination, including but not limited to Luteinizing Hormone (LH), Follicle Stimulating Hormone (FSH), and Estradiol. For instance, a surge in LH can be used to identify the transition point (ovulation) between the phases.

In some embodiments, the menstrual phase is determined based on the subject's reported clinical history, specifically the date of the Last Menstrual Period (LMP). In subjects with regular cycles (e.g., 28 days), the proliferative phase is typically defined as days 1 to 14, while the secretory phase is defined as days 15 to 28. While this method is less invasive, it can be combined with other methods for increased precision. The phase can also be assessed by tracking physiological changes, such as Basal Body Temperature (BBT), which typically rises by about 0.5° F. to 1.0° F. after ovulation due to thermogenic effects of progesterone, marking the onset of the secretory phase. Furthermore, the physical characteristics of cervical mucus can be evaluated; the proliferative phase is often characterized by abundant, clear, and stretchy mucus (spinnbarkeit) due to estrogen dominance, whereas the secretory phase is characterized by thick, opaque, and cellular mucus.

Alternatively, the phase of the cycle can be determined histologically, particularly when the sample obtained is an endometrial biopsy or uterine tissue. This approach, often referred to as “endometrial dating,” involves the microscopic examination of the tissue for phase-specific morphological features. For example, tissue in the proliferative phase exhibits mitotically active glandular epithelium and pseudostratified nuclei, whereas tissue in the secretory phase exhibits subnuclear vacuoles, stromal edema, and eventually pre-decidual changes. Standardized criteria, such as the Noyes criteria, can be employed by a pathologist to definitively categorize the tissue sample itself, thereby ensuring the microbiome data is mapped to the correct phase-specific bacterial panel without the need for a separate blood test.

In some embodiments, non-invasive imaging techniques can be employed to determine the cycle phase. Transvaginal ultrasonography can measure endometrial thickness and texture, as well as ovarian follicle status. The proliferative phase is typically associated with a “triple-line” endometrial pattern and the presence of a developing dominant follicle, while the secretory phase is associated with a hyperechoic, homogenous endometrium and the presence of a corpus luteum. The use of urine-based ovulation predictor kits (detecting the LH surge) or fertility monitors measuring urinary metabolites of estrogen and progesterone (e.g., pregnanediol-3-glucuronide) also falls within the scope of determining the cycle phase for the purposes of the present disclosure.

### 6.2.3 Panel of Bacterial Taxa

Disclosed herein are curated panels of bacterial taxa whose relative abundance is differentially associated with endometriosis. The panel can comprise specific genera, species, or strains (defined by the V4 region of specific 16S rRNA sequences).

### 6.2.3.1 Proliferative Phase Panel

In embodiments where the subject is in the proliferative phase of the menstrual cycle, the diagnostic method relies on quantifying the relative abundance of a specific panel of bacterial taxa that the inventors have discovered are differentially enriched or depleted in subjects with endometriosis during this specific hormonal window. Unlike existing methods that rely on general markers of vaginal dysbiosis (e.g., generic Gardnerella load), the present disclosure utilizes a high-definition feature set comprising at least one taxon selected from the group consisting of: Fenollaria, Anaeroglobus, Anaerococcus, Coprococcus, Prevotella, Varibaculum, Corynebacterium, Thalassobacillus, Staphylococcus, Priestia, Butyricimonas, Finegoldia, Mobiluncus, Cutibacterium, Peptoniphilus, Veillonella, and Gardnerella.

Expressly contemplated herein are various permutations and combinations of these taxa to form the diagnostic signature. The panel is not limited to the use of all 17 genera; rather, it may comprise a subset that is sufficient to achieve a classification accuracy (e.g., AUC of 0.70 or higher). In some embodiments, the panel comprises at least 2, 3, 4, 5, 6, 7, 8, 9, 10, 11, 12, 13, 14, 15, 16, or all 17 of said bacterial taxa. The panel can comprise 2 of said bacterial taxa. The panel can comprise 3 of said bacterial taxa. The panel can comprise 4 of said bacterial taxa. The panel can comprise 5 of said bacterial taxa. The panel can comprise 6 of said bacterial taxa. The panel can comprise 7 of said bacterial taxa. The panel can comprise 8 of said bacterial taxa. The panel can comprise 9 of said bacterial taxa. The panel can comprise 10 of said bacterial taxa. The panel can comprise 11 of said bacterial taxa. The panel can comprise 12 of said bacterial taxa. The panel can comprise 13 of said bacterial taxa. The panel can comprise 14 of said bacterial taxa. The panel can comprise 15 of said bacterial taxa. The panel can comprise 16 of said bacterial taxa. The panel can comprise 17 of said bacterial taxa. In other embodiments, the panel consists essentially of at least one of said bacterial taxa, meaning that while other bacteria may be present in the sequencing data, the classification decision is primarily driven by the relative abundances of these specific taxa. The panel can consist essentially of 2 of said bacterial taxa. The panel can consist essentially of 3 of said bacterial taxa. The panel can consist essentially of 4 of said bacterial taxa. The panel can consist essentially of 5 of said bacterial taxa. The panel can consist essentially of 6 of said bacterial taxa. The panel can consist essentially of 7 of said bacterial taxa. The panel can consist essentially of 8 of said bacterial taxa. The panel can consist essentially of 9 of said bacterial taxa. The panel can consist essentially of 10 of said bacterial taxa. The panel can consist essentially of 11 of said bacterial taxa. The panel can consist essentially of 12 of said bacterial taxa. The panel can consist essentially of 13 of said bacterial taxa. The panel can consist essentially of 14 of said bacterial taxa. The panel can consist essentially of 15 of said bacterial taxa. The panel can consist essentially of 16 of said bacterial taxa. The panel can consist essentially of 17 of said bacterial taxa. The classifier can also utilize pairs of taxa (e.g., a ratio of Fenollaria to Lactobacillus) or complex multi-variable patterns involving 5 to 10 of the listed genera.

A particularly unexpected aspect of the present disclosure is the inclusion of taxa traditionally associated with the gut microbiome, specifically Coprococcus and Butyricimonas. The detection of these genera in the uterine environment supports a mechanism involving the “gut-uterine axis,” potentially occurring via bacterial translocation or retrograde transport. The inclusion of Coprococcus (a butyrate producer typically found in the colon) and Butyricimonas in the uterine diagnostic panel represents a significant departure from conventional gynecological diagnostics that focus solely on vaginal flora. Accordingly, in some embodiments, the panel expressly comprises at least one gut-associated taxon selected from Coprococcus and Butyricimonas, optionally in combination with one or more vaginal-associated taxa such as Gardnerella or Prevotella. This combination of gut and vaginal signatures provides a multi-system view of the dysbiosis associated with endometriosis. In some embodiments, the panel of taxa that forms the diagnostic signature comprises Coprococcus. In some embodiments, the panel comprises Butyricimonas. In some embodiments, the panel comprises Coprococcus and Butyricimonas. In some embodiments, the panel comprises Gardnerella. In some embodiments, the panel comprises Prevotella. In some embodiments, the panel comprises Gardnerella and Prevotella.

While characterization at the genus level provides a robust diagnostic signal, methods provided herein also encompass assessing the microbiome at the species or strain level. In some embodiments, the panel of bacterial taxa described above is defined at species level. In some embodiments, the panel of bacterial taxa comprises at least one taxon selected from the group consisting of: Staphylococcus aureus, Fenollaria massiliensis, Priestia megaterium, Coprococcus catus, Butyricimonas faecihominis, Anaeroglobus geminatus, Anaerococcus octavius, Prevotella corporis, Varibaculum anthropi, Corynebacterium urealyticum, Thalassobacillus hwangdonensis, Corynebacterium tuberculostearicum, Staphylococcus intermedius, Finegoldia magna, Mobiluncus curtisii, Cutibacterium namnetense, Peptoniphilus harei, Priestia aryabhattai, Veillonella atypica, Prevotella timonensis, Prevotella bivia, and Gardnerella vaginalis. In some embodiments, the panel comprises at least one of Coprococcus catus and Butyricimonas faecihominis; and at least one of Gardnerella vaginalis, Prevotella corporis, Prevotella timonensis, and Prevotella bivia.

In some embodiments, the panel of bacterial taxa described above is defined not merely by genus, but by the V4 region of specific 16S rRNA gene sequences corresponding to distinct Operational Taxonomic Units (OTUs) or Amplicon Sequence Variants (ASVs).

In some embodiments, the bacterial taxa comprises at least one taxon selected from the group consisting of the taxa listed below, in which sequence in the parentheses correspond to the V4 region of their 16S rRNA gene sequence: Staphylococcus sp.1 (e.g., SEQ ID NO:3), Fenollaria sp.1 (e.g., SEQ ID NO:4), Priestia sp.1 (e.g., SEQ ID NO:5), Coprococcus sp.1 (e.g., SEQ ID NO:6), Butyricimonas sp.1 (e.g., SEQ ID NO:7), Anaeroglobus sp.1 (e.g., SEQ ID NO:8), Anaerococcus sp.1 (e.g., SEQ ID NO:9), Prevotella sp.1 (e.g., SEQ ID NO: 10), Varibaculum sp.1 (e.g., SEQ ID NO:11), Corynebacterium sp.1 (e.g., SEQ ID NO: 12), Thalassobacillus sp.1 (e.g., SEQ ID NO: 13), Corynebacterium sp.2 (e.g., SEQ ID NO: 14), Staphylococcus sp.2 (e.g., SEQ ID NO:15), Finegoldia sp.1 (e.g., SEQ ID NO: 16), Mobiluncus sp.1 (e.g., SEQ ID NO:17), Cutibacterium sp.1 (e.g., SEQ ID NO:18), Peptoniphilus sp.1 (e.g., SEQ ID NO:19), Priestia sp.2 (e.g., SEQ ID NO:20), Veillonella sp.1 (e.g., SEQ ID NO:21), Prevotella sp.2 (e.g., SEQ ID NO:22), Prevotella sp.3 (e.g., SEQ ID NO:23), and Gardnerella sp.1 (e.g., SEQ ID NO:24). In some embodiments, the panel comprises at least one of Coprococcus sp.1 (SEQ ID NO:6) and Butyricimonas sp.1 (SEQ ID NO:7); and at least one of Gardnerella sp.1 (SEQ ID NO:24), Prevotella sp.1 (SEQ ID NO:10), Prevotella sp.2 (SEQ ID NO:22), and Prevotella sp.3 (SEQ ID NO:23).

To account for natural evolutionary divergence and sequencing platform variations, the present disclosure is not limited to the exact sequences provided herein. It is well understood in the art that bacterial 16S sequences may vary slightly between strains of the same species due to natural evolutionary divergence. Therefore, the present disclosure covers taxa identified by a sequence having at least 95%, at least 95.5%, at least 96%, at least 96.5%, at least 97%, at least 97.5%, at least 98%, at least 98.5%, at least 99%, at least 99.5%, or 100% sequence identity to the reference SEQ ID NOs provided herein.

For example, the panel can include taxa defined by the V4 region of 16S rRNA gene sequences corresponding to: Staphylococcus sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:3), Fenollaria sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:4), Priestia sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:5), Coprococcus sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:6), Butyricimonas sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:7), Anaeroglobus sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:8), Anaerococcus sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:9), Prevotella sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:10), Varibaculum sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:11), Corynebacterium sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:12), Thalassobacillus sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:13), Corynebacterium sp.2 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:14), Staphylococcus sp.2 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:15), Finegoldia sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:16), Mobiluncus sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:17), Cutibacterium sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:18), Peptoniphilus sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:19), Priestia sp.2 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:20), Veillonella sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:21), Prevotella sp.2 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:22), Prevotella sp.3 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:23), and Gardnerella sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:24). In some embodiments, the strain is defined by the V4 region of its 16S rRNA gene having at least 95% sequence identity to the recited sequence. In some embodiments, the strain is defined by the V4 region of its 16S rRNA gene having at least 95.5% sequence identity to the recited sequence. In some embodiments, the strain is defined by the V4 region of its 16S rRNA gene having at least 96% sequence identity to the recited sequence. In some embodiments, the strain is defined by the V4 region of its 16S rRNA gene having at least 96.5% sequence identity to the recited sequence. In some embodiments, the strain is defined by the V4 region of its 16S rRNA gene having at least 97% sequence identity to the recited sequence. In some embodiments, the strain is defined by the V4 region of its 16S rRNA gene having at least 97.5% sequence identity to the recited sequence. In some embodiments, the strain is defined by the V4 region of its 16S rRNA gene having at least 98% sequence identity to the recited sequence. In some embodiments, the strain is defined the V4 region of by its 16S rRNA gene having at least 98.5% sequence identity to the recited sequence. In some embodiments, the strain is defined by the V4 region of its 16S rRNA gene having at least 99% sequence identity to the recited sequence. In some embodiments, the strain is defined by the V4 region of its 16S rRNA gene having at least 99.5% sequence identity to the recited sequence. In some embodiments, the strain is defined by the V4 region of its 16S rRNA gene having 100% sequence identity to the recited sequence.

Expressly contemplated herein are various permutations and combinations of these taxa to form the diagnostic signature. The panel is not limited to the use of all 22 strains; rather, it may comprise a subset that is sufficient to achieve a classification accuracy (e.g., AUC) of at least 0.70. In some embodiments, the panel comprises at least 2, 3, 4, 5, 6, 7, 8, 9, 10, 11, 12, 13, 14, 15, 16, 17, 18, 19, 20, 21, or 22 of said bacterial taxa. The panel can comprise 2 of said bacterial taxa. The panel can comprise 3 of said bacterial taxa. The panel can comprise 4 of said bacterial taxa. The panel can comprise 5 of said bacterial taxa. The panel can comprise 6 of said bacterial taxa. The panel can comprise 7 of said bacterial taxa. The panel can comprise 8 of said bacterial taxa. The panel can comprise 9 of said bacterial taxa. The panel can comprise 10 of said bacterial taxa. The panel can comprise 11 of said bacterial taxa. The panel can comprise 12 of said bacterial taxa. The panel can comprise 13 of said bacterial taxa. The panel can comprise 14 of said bacterial taxa. The panel can comprise 15 of said bacterial taxa. The panel can comprise 16 of said bacterial taxa. The panel can comprise 17 of said bacterial taxa. The panel can comprise 18 of said bacterial taxa. The panel can comprise 19 of said bacterial taxa. The panel can comprise 20 of said bacterial taxa. The panel can comprise 21 of said bacterial taxa. The panel can comprise 22 of said bacterial taxa. In other embodiments, the panel consists essentially of at least one of said bacterial taxa, meaning that while other bacteria may be present in the sequencing data, the classification decision is primarily driven by the relative abundances of the specific taxon. The panel can consist essentially of 2 of said bacterial taxa. The panel can consist essentially of 3 of said bacterial taxa. The panel can consist essentially of 4 of said bacterial taxa. The panel can consist essentially of 5 of said bacterial taxa. The panel can consist essentially of 6 of said bacterial taxa. The panel can consist essentially of 7 of said bacterial taxa. The panel can consist essentially of 8 of said bacterial taxa. The panel can consist essentially of 9 of said bacterial taxa. The panel can consist essentially of 10 of said bacterial taxa. The panel can consist essentially of 11 of said bacterial taxa. The panel can consist essentially of 12 of said bacterial taxa. The panel can consist essentially of 13 of said bacterial taxa. The panel can consist essentially of 14 of said bacterial taxa. The panel can consist essentially of 15 of said bacterial taxa. The panel can consist essentially of 16 of said bacterial taxa. The panel can consist essentially of 17 of said bacterial taxa. The panel can consist essentially of 18 of said bacterial taxa. The panel can consist essentially of 19 of said bacterial taxa. The panel can consist essentially of 20 of said bacterial taxa. The panel can consist essentially of 21 of said bacterial taxa. The panel can consist essentially of 22 of said bacterial taxa. The classifier can also utilize pairs of taxa (e.g., a ratio of Fenollaria to Lactobacillus) or complex multi-variable patterns involving 5 to 10 of the listed genera.

### 6.2.3.2 Secretory Phase Panel

In embodiments where the subject is in the secretory phase of the menstrual cycle, the diagnostic method relies on quantifying the relative abundance of a specific panel of bacterial taxa that the inventors have discovered are differentially enriched or depleted in subjects with endometriosis during this specific hormonal window. Unlike existing methods that rely on general markers of vaginal dysbiosis (e.g., generic Gardnerella load), the present disclosure utilizes a high-definition feature set comprising at least one taxon selected from the group consisting of: Ureaplasma, Niallia, Murdochiella, Gardnerella, Lactobacillus, Lawsonella, Corynebacterium, Priestia, Finegoldia, and Dialister.

Expressly contemplated herein are various permutations and combinations of these taxa to form the diagnostic signature. The panel is not limited to the use of all 10 genera; rather, it may comprise a subset that is sufficient to achieve a classification accuracy (e.g., AUC) of at least 0.70. In some embodiments, the panel comprises at least 2, 3, 4, 5, 6, 7, 8, 9, or all 10 of said bacterial taxa. The panel can comprise 2 of said bacterial taxa. The panel can comprise 3 of said bacterial taxa. The panel can comprise 4 of said bacterial taxa. The panel can comprise 5 of said bacterial taxa. The panel can comprise 6 of said bacterial taxa. The panel can comprise 7 of said bacterial taxa. The panel can comprise 8 of said bacterial taxa. The panel can comprise 9 of said bacterial taxa. The panel can comprise 10 of said bacterial taxa. In other embodiments, the panel consists essentially of at least one of said bacterial taxa, meaning that while other bacteria may be present in the sequencing data, the classification decision is primarily driven by the relative abundances of these specific taxa. The panel can consist essentially of 2 of said bacterial taxa. The panel can consist essentially of 3 of said bacterial taxa. The panel can consist essentially of 4 of said bacterial taxa. The panel can consist essentially of 5 of said bacterial taxa. The panel can consist essentially of 6 of said bacterial taxa. The panel can consist essentially of 7 of said bacterial taxa. The panel can consist essentially of 8 of said bacterial taxa. The panel can consist essentially of 9 of said bacterial taxa. The panel can consist essentially of 10 of said bacterial taxa. The classifier can also utilize pairs of taxa (e.g., a ratio of Ureaplasma to Lactobacillus) or complex multi-variable patterns involving 5 to 10 of the listed genera.

While characterization at the genus level provides a robust diagnostic signal, methods provided herein also encompass assessing the microbiome at the species or strain level. In some embodiments, the panel of bacterial taxa described above is defined not merely by genus, but at the species level. In some embodiments, the panel of bacterial taxa comprises at least one taxon selected from the group consisting of Ureaplasma urealyticum, Niallia oryzisoli, Murdochiella asaccharolytica, Gardnerella vaginalis, Lactobacillus iners, Lactobacillus jensenii, Lawsonella clevelandensis, Corynebacterium kroppenstedtii, Priestia megaterium, Lactobacillus crispatus, Finegoldia magna, and Dialister hominis, Lactobacillus vaginalis, and Ureaplasma parvum.

In some embodiments, the panel of bacterial taxa described above is defined by the V4 region of specific 16S rRNA gene sequences corresponding to distinct OTUs or ASVs.

In some embodiments, the bacterial taxa comprises at least one taxon selected from the group consisting of the taxa listed below, in which sequence in the parentheses correspond to the V4 region of their 16S rRNA gene sequence: Ureaplasma sp.1 (e.g., SEQ ID NO:25), Niallia sp.1 (e.g., SEQ ID NO:26), Murdochiella sp.1 (e.g., SEQ ID NO:27), Gardnerella sp.1 (e.g., SEQ ID NO:24), Lactobacillus sp.1 (e.g., SEQ ID NO:28), Lactobacillus sp.2 (e.g., SEQ ID NO:29), Lawsonella sp.1 (e.g., SEQ ID NO:30), Corynebacterium sp.3 (e.g., SEQ ID NO:31), Priestia sp.1 (e.g., SEQ ID NO:5), Lactobacillus sp.3 (e.g., SEQ ID NO:32), Finegoldia sp.1 (e.g, SEQ ID NO:16), Dialister sp.1 (e.g., SEQ ID NO:33), Lactobacillus sp.4 (e.g., SEQ ID NO:34), and Ureaplasma sp.2 (e.g., SEQ ID NO:35).

To account for natural evolutionary divergence and sequencing platform variations, the present disclosure is not limited to the exact sequences provided herein. It is well understood in the art that bacterial 16S sequences may vary slightly between strains of the same species due to natural evolutionary divergence. Therefore, the present disclosure covers taxa identified by a sequence having at least 95%, at least 95.5%, at least 96%, at least 96.5%, at least 97%, at least 97.5%, at least 98%, at least 98.5%, at least 99%, at least 99.5%, or 100% sequence identity to the reference SEQ ID NOs provided herein.

For example, the panel can include taxa defined by the V4 region of 16S rRNA gene sequences corresponding to: Ureaplasma sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:25), Niallia sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:26), Murdochiella sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:27), Gardnerella sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:24), Lactobacillus sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:28), Lactobacillus sp.2 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:29), Lawsonella sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:30), Corynebacterium sp.3 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:31), Priestia sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:5), Lactobacillus sp.3 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:32), Finegoldia sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:16), Dialister sp.1 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:33), Lactobacillus sp.4 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:34), Ureaplasma sp.2 (having at least 95%, at least 95.5%, at least 96%, at least 96.5%, at least 97%, at least 97.5%, at least 98%, at least 98.5%, at least 99%, at least 99.5%, or 100% sequence identity to SEQ ID NO:35). In some embodiments, the strain is defined by the V4 region of its 16S rRNA gene having at least 95% sequence identity to the recited sequence. In some embodiments, the strain is defined by the V4 region of its 16S rRNA gene having at least 95.5% sequence identity to the recited sequence. In some embodiments, the strain is defined by the V4 region of its 16S rRNA gene having at least 96% sequence identity to the recited sequence. In some embodiments, the strain is defined by the V4 region of its 16S rRNA gene having at least 96.5% sequence identity to the recited sequence. In some embodiments, the strain is defined by the V4 region of its 16S rRNA gene having at least 97% sequence identity to the recited sequence. In some embodiments, the strain is defined by the V4 region of its 16S rRNA gene having at least 97.5% sequence identity to the recited sequence. In some embodiments, the strain is defined by the V4 region of its 16S rRNA gene having at least 98% sequence identity to the recited sequence. In some embodiments, the strain is defined by the V4 region of its 16S rRNA gene having at least 98.5% sequence identity to the recited sequence. In some embodiments, the strain is defined by the V4 region of its 16S rRNA gene having at least 99% sequence identity to the recited sequence. In some embodiments, the strain is defined by the V4 region of its 16S rRNA gene having at least 99.5% sequence identity to the recited sequence. In some embodiments, the strain is defined by the V4 region of its 16S rRNA gene having 100% sequence identity to the recited sequence.

Expressly contemplated herein are various permutations and combinations of these taxa to form the diagnostic signature. The panel is not limited to the use of all 14 strains; rather, it may comprise a subset that is sufficient to achieve a classification accuracy (e.g., AUC) of at least 0.70. In some embodiments, the panel comprises at least 2, 3, 4, 5, 6, 7, 8, 9, 10, 11, 12, 13, or 14 of said bacterial taxa. The panel can comprise 2 of said bacterial taxa. The panel can comprise 3 of said bacterial taxa. The panel can comprise 4 of said bacterial taxa. The panel can comprise 5 of said bacterial taxa. The panel can comprise 6 of said bacterial taxa. The panel can comprise 7 of said bacterial taxa. The panel can comprise 8 of said bacterial taxa. The panel can comprise 9 of said bacterial taxa. The panel can comprise 10 of said bacterial taxa. The panel can comprise 11 of said bacterial taxa. The panel can comprise 12 of said bacterial taxa. The panel can comprise 13 of said bacterial taxa. The panel can comprise 14 of said bacterial taxa. In other embodiments, the panel consists essentially of at least one of said bacterial taxa, meaning that while other bacteria may be present in the sequencing data, the classification decision is primarily driven by the relative abundances of the specific taxon. The panel can consist essentially of 2 of said bacterial taxa. The panel can consist essentially of 3 of said bacterial taxa. The panel can consist essentially of 4 of said bacterial taxa. The panel can consist essentially of 5 of said bacterial taxa. The panel can consist essentially of 6 of said bacterial taxa. The panel can consist essentially of 7 of said bacterial taxa. The panel can consist essentially of 8 of said bacterial taxa. The panel can consist essentially of 9 of said bacterial taxa. The panel can consist essentially of 10 of said bacterial taxa. The panel can consist essentially of 11 of said bacterial taxa. The panel can consist essentially of 12 of said bacterial taxa. The panel can consist essentially of 13 of said bacterial taxa. The panel can consist essentially of 14 of said bacterial taxa. The classifier can also utilize pairs of taxa (e.g., a ratio of Ureaplasma to Lactobacillus) or complex multi-variable patterns involving 5 to 10 of the listed genera.

### 6.2.4 Functional Dysbiosis Score (FDS)

In some embodiments, the microbiome features comprise Lactobacillus dysbiosis. As used herein consistently with its understanding in the art, the term “Lactobacillus dysbiosis” refers to a disruption in the normal population of Lactobacillus species in the microbiome, typically resulting in a reduction of these beneficial bacteria. Lactobacillus species, such as Lactobacillus crispatus, Lactobacillus jensenii, Lactobacillus gasseri, and Lactobacillus iners produces lactic acid, which helps maintain a low vaginal pH (around 3.8 to 4.5). This acidic environment inhibits the growth of pathogenic bacteria and protects against infections.

In Lactobacillus dysbiosis, the levels of these beneficial bacteria drop below a heathy threshold, typically less than 90% of the total microbial population, leading to an imbalance in the microbiome, which often results in the overgrowth of other microorganisms, such as anaerobic bacteria (e.g., Gardnerella vaginalis), yeasts, or other opportunistic pathogens that thrive in a less acidic environment.

In addition to the relative abundance of selected bacterial taxa, the methods herein further utilize a calculated metric termed the “Functional Dysbiosis Score” or FDS to capture the overall disruption of the microbiome. The FDS integrates the depletion of protective species with the enrichment of pathogenic species into a single feature for the machine learning classifier.

In one embodiment, the FDS is calculated according to the formula below:

FDS=0.5×(1−A_{Lacto})+10×Aₚₐₜₕₒ, wherein A_{Lacto} is the relative abundance of the genus Lactobacillus (or specific protective species thereof), and Aₚₐₜₕₒ is the cumulative relative abundance of a plurality of pathogenic taxa.

In some embodiments, the plurality of pathogenic taxa used to calculate Aₚₐₜₕₒ comprises one or more, or a combination of, genera known to be associated with bacterial vaginosis or inflammation, including but not limited to: Gardnerella, Prevotella, Anaerococcus, Streptococcus, Megasphaera, Mobiluncus, Sneathia, Atopobium, Peptoniphilus, Mycoplasmoides, Ureaplasma, Bacteroides, Peptostreptococcus, and Dialister. In some embodiments, the plurality of pathogenic taxa used to calculate Aₚₐₜₕₒ consists of: Gardnerella, Prevotella, Anaerococcus, Streptococcus, Megasphaera, Mobiluncus, Sneathia, Atopobium, Peptoniphilus, Mycoplasmoides, Ureaplasma, Bacteroides, Peptostreptococcus, and Dialister

Alternatives to the formula above are also contemplated. For example, the coefficients (0.5 and 10) can be adjusted based on the specific sequencing platform used. In other embodiments, the FDS can be calculated as a simple ratio of Aₚₐₜₕₒ/A_{Lacto}, or as a log-transformed ratio.

### 6.2.5 Trained Machine Learning Classifier

Methods disclosed herein use a trained machine learning classifier to resolve the complex, non-linear relationships between microbial abundance and the presence of endometriosis. Unlike traditional statistical tests (e.g., t-tests or Wilcoxon rank-sum tests) that evaluate biomarkers in isolation, machine learning classifiers can detect high-order interactions between multiple bacterial taxa. The present disclosure leverages this capability to transform multidimensional microbiome sequencing data into a single, actionable diagnostic score.

In some embodiments, the classifier is a Random Forest classifier. Random Forest is an ensemble learning method that operates by constructing a multitude of decision trees at training time. It is particularly suitable for microbiome data due to its inherent ability to handle high-dimensional, sparse data (where many taxa have zero counts) and its resistance to overfitting. By averaging the results of many uncorrelated trees, the Random Forest minimizes the variance of the model. Technical details regarding the implementation of Random Forests can be found in Breiman, Machine learning 45.1 (2001): 5-32, which is incorporated herein by reference. In specific embodiments, the Random Forest is configured with specific hyperparameters, such as the number of trees (ntree, e.g., 500 or 1000) and the number of variables tried at each split (mtry), optimized to maximize the Area Under the Receiver Operating Characteristic Curve (AUC-ROC).

However, methods disclosed herein are not limited to Random Forest. The diagnostic platform can utilize a variety of supervised learning algorithms capable of binary or multi-class classification. Alternative machine learning algorithms that can be used include, for example:

Support Vector Machines (SVM): SVMs are effective in high-dimensional spaces and work by finding the hyperplane that best separates the classes (Endometriosis vs. Control). The invention contemplates the use of SVMs with various kernels, including linear, polynomial, and Radial Basis Function (RBF) kernels, to map the microbial data into higher-dimensional feature spaces. See Cortes and Vapnik, Machine learning 20.3 (1995): 273-297.

TabPFN (Tabular Prior-Data Fitted Network): The classifier can utilize a Transformer-based model designed specifically for tabular data, such as TabPFN. TabPFN utilizes a Transformer architecture pre-trained on a large corpus of synthetic priors to approximate Bayesian inference in a single forward pass. This method is particularly advantageous for microbiome datasets which often possess limited sample sizes relative to feature count (high-dimensional, small-n), as it requires no hyperparameter tuning and provides robustness against overfitting. See Hollmann et al., Nature 637 (2025): 319-326.

Logistic Regression and Regularized Regression: This includes models utilizing LASSO (Least Absolute Shrinkage and Selection Operator), Ridge, or Elastic Net regularization. These methods are particularly useful for feature selection, as LASSO (L1 regularization) can shrink the coefficients of non-predictive taxa to zero, effectively removing noise from the model. See Tibshirani, Journal of the Royal Statistical Society: Series B 58.1 (1996): 267-288.

Gradient Boosting Machines (GBM): Algorithms such as XGBoost (eXtreme Gradient Boosting) and LightGBM build models sequentially, with each new model attempting to correct the errors of the previous ones. These are highly effective for tabular microbiome data and often provide state-of-the-art performance. See Chen and Guestrin, Proceedings of the 22nd ACM SIGKDD (2016).

Neural Networks and Deep Learning: The classifier can employ architectures such as Multi-layer Perceptrons (MLP), Convolutional Neural Networks (CNN) adapted for 1D sequence data, or Deep Belief Networks. These models are capable of learning complex, hierarchical representations of the microbiome data.

Decision Trees: Simple interpretable models such as C4.5 or CART (Classification and Regression Trees) may be used, particularly when model explainability is a priority for clinical adoption.

k-Nearest Neighbors (k-NN): A non-parametric method used for classification where the input consists of the k closest training examples in the feature space.

Naïve Bayes classifiers: Probabilistic classifiers based on applying Bayes' theorem with strong (naïve) independence assumptions between the features.

The classifier is trained using a labeled dataset of microbiome profiles from subjects with confirmed endometriosis (cases) and confirmed absence of endometriosis (controls). In some embodiment, the training process employs repeated random subsampling cross-validation to ensure robustness and assess the stability of the selected biomarkers. For example, the dataset can be randomly split into a training set (comprising, e.g., 70%, 75%, 80%, or 90% of the data) and a testing set (comprising, e.g., 30%, 25%, 20%, or 10% of the data) over multiple iterations (e.g., 10, 20, 50, 100, or 1000 iterations). In each iteration, the model is trained on the training set and evaluated on the held-out testing set. The final classification output can be derived from an average, consensus, or majority vote of these iterations, thereby providing a confidence interval for the diagnosis.

Prior to training, features (bacterial taxa) are preferably selected or filtered using Multivariable Association Analysis to identify the most relevant biological signals. In some embodiments, the analysis is performed using MaAsLin2 (Microbiome Multivariable Associations with Linear Models). MaAsLin2 is a comprehensive statistical framework that determines multivariable associations between clinical metadata and microbial omics features.

See Mallick et al., PLoS computational biology 17.11 (2021): e1009442.

The use of MaAsLin2 or similar multivariable frameworks (e.g., multivariate logistic regression) allows for the control of confounding variables. Endometriosis patients often differ from controls in demographic factors such as age (due to diagnostic delay) and Body Mass Index (BMI). By including these confounders as fixed effects in the linear model, the method ensures that the identified biomarkers (e.g., Fenollaria, Priestia) are specifically associated with the disease pathology. In some embodiments, the analysis is controlled for confounders such as age, BMI, ethnicity, parity, menstrual cycle regularity, and hormonal contraceptive use. In some embodiments, the analysis is controlled for age. In some embodiments, the analysis is controlled for BMI. In some embodiments, the analysis is controlled for age and BMI. In some embodiments, the analysis is further controlled for ethnicity, parity, menstrual cycle regularity, and/or hormonal contraceptive use.

### 6.2.6 Sequencing and Data Processing

In some embodiments, the methods provided herein comprise isolating nucleic acids from the biological sample prior to sequencing. Nucleic acids (e.g., DNA and/or RNA) can be purified from the sample using standard molecular biology techniques known in the art. These techniques can include methods described in, e.g., Sambrook, J., Molecular Cloning: A Laboratory Manual, Cold Spring Harbor Laboratory Press; 4th edition (Jun. 15, 2012), and Ausubel et al., eds., Short Protocols in Molecular Biology, 5th ed., John Wiley & Sons, Inc., Hoboken, N.J. (2002). In some embodiments, the method comprises preparing microorganism DNA from a sample, such as a uterine or vaginal sample. In some embodiments, this process involves specific lysis steps to break down the robust cell walls of Gram-positive bacteria (e.g., using enzymatic lysis with lysozyme or mutanolysin, or mechanical bead-beating) while preserving the integrity of the genomic DNA. The nucleic acids can also be obtained through in vitro amplification methods, including PCR, as those described herein and in Sambrook and Ausubel. In some embodiments, nucleic acids are quantified without amplification.

The methods provided herein utilize nucleic acid sequencing to identify the bacterial taxa. In some embodiments, the method comprises obtaining sequence reads of the microorganism nucleic acids. DNA sequencing can be performed using various advanced methodologies. Traditional and Next-Generation Sequencing (NGS) or high-throughput sequencing technologies, such as Illumina, Life Technologies, and Roche 454 sequencing systems, have been widely used. These platforms enable large-scale sequencing, providing the ability to generate sequence data from numerous reads. For example: Roche 454 utilizes emulsion PCR to immobilize DNA fragments on beads. Light emission during nucleotide incorporation is measured to determine the sequence. Illumina technology involves attaching DNA to a surface, amplifying it using bridge PCR, and sequencing with reversible terminators tagged with fluorescent dyes. Popular Illumina systems suitable for the present invention include the iSeq, MiniSeq, MiSeq, NextSeq, HiSeq, and NovaSeq systems. Life Technologies SOLiD uses sequencing by hybridization, involving a pool of labeled oligonucleotides to detect the DNA sequence.

Beyond these traditional methods, newer DNA sequencing technologies have emerged, offering greater capabilities, including Single-Molecule Real-Time (SMRT) sequencing, nanopore sequencing, multi-omics sequencing, high-throughput short-read sequencers, single-molecule proteomic sequencing, and ultra-high throughput sequencing.

SMRT sequencing is a sequencing-by-synthesis technology developed by PacBio (Pacific Biosciences) which allows for real-time observation of nucleotide incorporation. PacBio has also introduced high-throughput library preparation kits optimized for their sequencing system. Nanopore Sequencing is developed by Oxford Nanopore Technologies (ONT), which uses nanopores to read DNA sequences as they pass through a membrane. The PromethION 2 Integrated (P2i) is a desktop sequencing device capable of real-time base calling and post-run analysis without the need for external computing resources. Multi-omics sequencing combines DNA, RNA, and protein sequencing within a single sample or cell, which provides robust insights into the relationships between various biological molecules. The developments in single-cell and spatial multi-omics methods have improved the resolution and accuracy of these analyses. High-throughput short-read sequencers, such as Element Biosciences' AVITI System and Singular Genomics' G4 Platform, provide cost-effective sequencing options and anticipate further technological advancements. Ultra-high throughput sequencing, such as MGI Tech's DNBSEQ-T20×2, is designed for ultra-high throughput processing, which is compatible with whole-genome, bisulfite, long-fragment, and single-cell sequencing technologies.

In some embodiments, methods provided herein comprise deep sequencing of the microorganism nucleic acids. Given the low microbial biomass in certain samples, such as uterine samples, compared to the gut or vagina, deep sequencing is critical to ensure detection of low-abundance taxa (e.g., Fenollaria or Priestia) that may drive the machine learning classifier. Deep sequencing can be used to quantify the number of copies of a particular sequence in a sample and then also be used to determine the relative abundance of different sequences in a sample. Deep sequencing refers to highly redundant sequencing of a nucleic acid sequence, for example such that the original number of copies of a sequence in a sample can be determined or estimated. The redundancy (i.e., depth) of the sequencing is determined by the length of the sequence to be determined (X), the number of sequencing reads (N), and the average read length (L). The redundancy is then calculated as N×L/X. In the methods provided herein, the sequencing depth can be, or be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 110, 120, 130, 150, 200, 300, 500, 700, 1000, 2000, 3000, 4000, 5000 or more. In specific embodiments relevant to clinical diagnostics, the depth is sufficient to generate at least 10,000, at least 50,000, or at least 100,000 reads per sample.

Sequencing can target specific regions, such as the 16S rRNA gene, to identify bacterial species, or it can involve whole-genome sequencing for a broader microbial profile. In some embodiments, methods provided herein comprise 16S rRNA deep sequencing reads. Sequence analysis of the 16S rRNA gene is widely used to identify bacterial species and perform taxonomic studies because this gene contains nine “hypervariable regions” (V1-V9) with significant sequence diversity among different bacterial species. These regions are key for species identification. Accordingly, the term “hypervariable regions” of the 16S rRNA gene refers to specific sequences within the 16S rRNA gene that allow for the identification of individual bacterial species or differentiation among a limited number of different species or genera. This method is particularly useful for studying bacterial diversity and composition in a given sample.

In some embodiments, the V4 region of the 16S rRNA gene is amplified. Exemplary primer sequences used in the amplification can include:
Forward Primer: 5′-TAATTGTGTGCCAGCmGCCGCGGTAA-3′ (SEQ ID NO:1)
Reverse Primer: 5′-TCAGCCGGACTAChvGGGTwTCTAAT-3′ (SEQ ID NO:2)

PCR conditions are optimized to minimize bias, using high-fidelity polymerases (e.g., Platinum SuperFi II) and optimized cycling parameters (e.g., 25-35 cycles).

Given the nature of uterine samples, host DNA contamination is a challenge. In some embodiments, the methods herein comprise a step of bioinformatically removing sequencing reads that map to a human reference genome (e.g., hg19, hg38, or T2T-CHM13). Software tools such as Bowtie2, BWA, or Hostile can be used for this filtering. Following decontamination, the sequence reads are mapped to determine the microbiome feature associated with endometriosis (e.g., presence, absence, abundance, or relative abundance of certain taxonomic groups) in the sample. Once raw sequencing data is generated, the sequence reads can be mapped to known sequences in genomic databases. Suitable algorithms for determining sequence identity and aligning the reads include BLAST (Basic Local Alignment Search Tool) and BLAST 2.0. These tools are publicly available through platforms like the National Center for Biotechnology Information (NCBI). During analysis, a subset of the reads is aligned to bacterial genomes or specific gene sequences associated with microbiome features indicative of endometriosis, such as the presence, absence, abundance, or relative abundance of a microbial biomarker. The reads are designated to particular bacterial species or genetic pathways based on the best alignment to database sequences (e.g., Greengenes, SILVA, RDP, or GTDB).

Assuming sufficient sequencing depth, the number of reads corresponding to specific microbiome features can be quantified. This quantity can be expressed either as an absolute value, such as the number of reads mapping to a specific bacterial genus, or as a relative abundance by comparing the number of reads for a given microbial feature against the total reads for the entire microbial domain (e.g., the total 16S rRNA V4 region sequence reads).

In some embodiments, these values are compared to predefined cut-off values or probability distributions characteristic of a microbiome associated with endometriosis. For instance, if the analysis indicates that a particular feature's relative abundance of 50% or more correlates with endometriosis, then finding a value above this threshold in the sample suggests a higher likelihood of an endometriosis-associated microbiome. Conversely, a relative abundance below this threshold would indicate a lower likelihood. By comparing the quantified features to the established disease signatures for endometriosis (e.g., the Proliferative or Secretory panels described herein), the method allows determination of the presence and likelihood of an individual having a microbiome profile indicative of the condition. By using deep sequencing data and comparing the microbial profile with established signatures, this method allows for accurate diagnosis and assessment of the risk of endometriosis. This comprehensive approach, which includes the sequencing and data analysis, supports tailored diagnostic and therapeutic strategies for endometriosis.

### 6.2.7 Biological Samples

In some embodiments, methods provided herein comprise obtaining a biological sample from the subject. The methods are particularly tailored for samples derived from the female reproductive tract, where the microbial signature of endometriosis is most pronounced. Upon analysis, the sample provides information about the tissue status or the health or diseased status of the organ or individual. Suitable sample types include, but are not limited to, cervicovaginal fluid, blood, vaginal mucosa, interstitial fluid, cervical secretion, uterine tissue, reproductive cells, cervical cells, endometrial cells, fallopian cells, ovarian cells, or natural flora found in the female reproductive tract. In some embodiments, the selection of the specific sample type depends on the clinical setting, balancing the need for proximity to the endometrial lesion with the invasiveness of the collection procedure.

In some embodiments, the sample is a uterine Samples: Uterine samples directly represent the endometrial environment where the pathology originates. In some embodiments, the sample comprises uterine tissue, which can be collected via minimally invasive procedures such as a Pipelle biopsy, curettage, or during a hysterectomy. In some embodiments, the sample comprises uterine fluid.

Endometrial tissue provides a comprehensive profile of both the tissue-adherent microbiome and the host cellular architecture. In some embodiments, the sample comprises endometrial cells isolated from the tissue matrix or fluid. Alternatively, the sample can comprise uterine lavage fluid, collected by flushing the uterine cavity with a sterile solution (e.g., saline) to capture the planktonic microbiome, or uterine fluid aspirated directly from the cavity using a specialized catheter.

Vaginal and cervical samples offer a less invasive alternative suitable for screening larger populations or for serial monitoring. In some embodiments, the sample comprises cervicovaginal fluid, vaginal mucus, or cervical mucus, which can be collected using swabs, wicking devices, or lavage. Importantly, the sample may also comprise vaginal mucosa, obtained by scrubbing the vaginal wall with a synthetic or flocked swab to collect the adherent biofilm. Cervical cells and secretions collected from the cervical os also serve as valuable proxies for the upper genital tract microbiome due to the continuum between the endocervix and the uterus.

The methods are applicable to a variety of other samples derived from the female reproductive tract or systemic circulation. For example, the method can be applied to menstrual effluent collected via a menstrual cup or specialized pad. Samples of peritoneal fluid, often collected during laparoscopic procedures, can provide insight into the microbiome of the pelvic cavity where ectopic lesions reside. Furthermore, blood or serum samples can be analyzed to detect circulating microbial DNA (bacteremia) or immune markers associated with bacterial translocation from the gut or uterus, offering a completely non-invasive screening option.

### 6.2.8 Subjects

Provided herein are methods for assessing the likelihood of a subject having endometriosis by characterizing the microbiome in a sample from the subject. In some embodiments, the subject is a female. In some embodiments, the subject is a human female. In some embodiments, the subject is an adolescent human female (e.g., aged 12-18 years). In some embodiments, the subject is an adult of reproductive age (e.g., aged 18-49 years). The machine learning classifier described herein can be adjusted for specific demographic variables, including the subject's age and BMI (e.g., Underweight, Normal, Overweight, Obese), to ensure the microbial signature is specific to the disease rather than a demographic artifact.

In some embodiments, the subject is suspected of having endometriosis. The methods are highly suitable for subjects presenting with one or more clinical indicators of endometriosis. Clinical indicators include, but are not limited to, intermenstrual bleeding, dysmenorrhea (painful menstruation), chronic pelvic pain, deep dyspareunia (pain during intercourse), dyschezia (painful defecation), dysuria (painful urination), lower abdominal pain, or fatigue. In embodiments regarding prognosis, the subject is known to have endometriosis, and the method is used to monitor disease progression or recurrence.

The methods provided herein are also effective in subjects who are asymptomatic, meaning they exhibit no overt physical symptoms of endometriosis (such as pain) at the time of assessment. In some embodiments, the subject presents with infertility of unknown origin as the sole indication. The microbial signature can detect “silent” endometriosis in these patients, allowing for earlier intervention that may preserve fertility. In other embodiments, the subject is at risk of developing endometriosis due to a family history of the disease.

### 6.2.9 Additional Methods of Assessing

In some embodiments, the microbiome-based methods disclosed herein can serve as a primary screening method for an initial risk assessment for endometriosis, allowing for the identification of patients who can benefit from further testing through established diagnostic techniques. Upon identification of an elevated risk (e.g., a positive classification output from the machine learning model), these subjects are recommended to undergo additional diagnostic tests to confirm the presence of endometriosis and determine the extent of the disease.

Common diagnostic approaches that can follow the present assessment include transvaginal ultrasound, Magnetic Resonance Imaging (MRI), and laparoscopy.

In some embodiments, the subjects can be further assessed by examining miRNA biomarkers or protein biomarkers to corroborate the microbiome findings. In some embodiments, the microbiome-based assessment described herein serves as a foundational screening or diagnostic step. To further increase sensitivity and specificity, particularly for early-stage disease or complex cases, the subject can be further assessed by measuring additional molecular biomarkers to corroborate the microbiome findings. By integrating microbial signatures with host-derived biomarkers (e.g., circulating miRNAs or proteins), the method achieves a “multi-modal” diagnostic power that exceeds the accuracy of either modality alone.

Accordingly, in some embodiments, the methods further comprise measuring the expression level of one or more microRNA (miRNA) biomarkers or protein biomarkers in a sample from the subject. These biomarkers can be analyzed in the same sample used for microbiome sequencing (e.g., cervicovaginal fluid or menstrual effluent) or in a separate sample (e.g., serum or plasma). The quantitative data from these host biomarkers can be input into the same machine learning classifier used for the microbiome features, or into a separate parallel classifier whose output is combined with the microbiome risk score.

Exemplary methods for miRNA and protein biomarker profiling for endometriosis are described in International Patent Application No. PCT/US2025/010377, filed Jan. 10, 2025, which is incorporated herein by reference in its entirety. As disclosed therein, specific panels of miRNAs and proteins have been identified that, when combined with clinical metadata, highly accurately predict the presence of endometriotic lesions.

In some embodiment, methods provided herein further comprise assessing miRNA biomarkers. In some embodiments, the miRNA biomarkers comprise miR-17-5p and/or miR-15b-5p. These miRNAs have been validated as differentially expressed in subjects with endometriosis. To ensure accurate quantification, the levels of these biomarkers are preferably normalized against an endogenous control, such as miR-92a-3p, which remains stable across disease states.

In some embodiment, methods provided herein further comprise assessing protein biomarkers. In some embodiments, the protein biomarkers comprise one or more of CA125 (Cancer Antigen 125), CA19-9 (Carbohydrate Antigen 19-9), and SHBG (Sex Hormone Binding Globulin). While CA125 alone lacks sensitivity for early-stage disease, its inclusion in a multi-analyte panel (specifically in combination with the miRNA and microbiome features) significantly enhances its predictive value. Additionally, measuring progesterone levels can serve a dual purpose: determining the menstrual phase (to select the correct microbiome panel, as described herein and serving as a feature in the multi-modal algorithm itself.

In embodiments where these host biomarkers are integrated with microbiome data, the diagnostic model is constructed using a robust training workflow designed to handle heterogeneous data types. In some embodiments, a binary classification model, such as a Random Forest, is trained using a unified feature matrix wherein the columns represent the combined features—comprising microRNA expression levels, protein concentrations, and clinical demographic information—and the rows represent individual patient samples. In some embodiments, the input feature set includes the normalized expression values of miR-17-5p and miR-15b-5p; the serum concentrations of CA125, CA19-9, SHBG, and Progesterone; and key clinical metadata variables including the subject's Age and Body Mass Index (BMI). This multi-dimensional matrix allows the algorithm to learn complex, non-linear interactions between the host's metabolic state (BI/Progesterone), immune response (miRNAs/Proteins), and microbial dysbiosis.

In some embodiments, the machine learning model can be implemented using standard data science libraries, such as the Python Scikit-learn package. The training process initiates by generating bootstrap samples of the training data to construct a multitude of independent decision trees. Each bootstrapped tree is trained on a random subset of the data created by sampling with replacement (bagging), which ensures that the model remains generalizable and resistant to overfitting. During the growth of each decision tree, the algorithm evaluates potential data splits at each node using a rigorous metric, such as Gini impurity or Information Gain, to determine the optimal threshold that best separates the binary classes (Endometriosis vs. Control). Each tree is grown independently to its maximum depth or until a pre-defined stopping criterion is met.

Once the ensemble of trees is fully constructed, the Random Forest generates a final prediction by aggregating the outputs of the individual trees. For a categorical classification (e.g., “Positive” or “Negative”), the final prediction is determined by a majority vote, wherein the class predicted by the greatest number of trees is selected. Furthermore, the model provides a granular risk assessment by calculating a prediction probability (e.g., “85% likelihood of Endometriosis”); this score is derived by averaging the class probabilities output by all trees in the forest. This probabilistic output allows for risk stratification, enabling clinicians to distinguish between high-confidence diagnoses and borderline cases that may require monitoring.

By combining the exogenous signal (uterine microbiome dysbiosis, e.g., Fenollaria and Priestia abundance) with the endogenous host response (miRNA dysregulation and inflammatory protein markers), the methods provided in present disclosure provides a comprehensive biological snapshot of the disease state, minimizing false negatives associated with single-analyte tests.

### 6.3 KITS AND SYSTEMS

In some embodiments, provided herein are kits and systems for assessing whether a subject has endometriosis. In some embodiments, the kits disclosed herein comprise a comprehensive system integrating physical reagents for generating high-resolution microbiome data with computational tools for interpreting that data. In some embodiments, the kit comprises a means for obtaining a dataset representing a plurality of nucleic acid sequences (e.g., NGS reagents). In some embodiments, the kit comprises a non-transitory computer-readable medium storing specific algorithmic instructions. In some embodiments, the kit comprises a means for obtaining a dataset representing a plurality of nucleic acid sequences (e.g., NGS reagents), and (2) a non-transitory computer-readable medium storing specific algorithmic instructions.

### 6.3.1 Physical Reagents and Sample Collection

In some embodiments, the means for obtaining a dataset comprises reagents configured for the targeted amplification and sequencing of bacterial nucleic acids. In some embodiments, the kit may comprise a primer set configured to amplify the V4 region of bacterial 16S rRNA. In some embodiments, the primer set consists of a forward primer comprising SEQ ID NO:1 and a reverse primer comprising SEQ ID NO:2. These primers can be lyophilized, in solution, or attached to a solid support. The primers can further comprise adapter sequences or unique molecular identifiers (UMIs) to facilitate Next-Generation Sequencing (NGS).

To facilitate deep sequencing, in some embodiments, the kit can contain components to facilitate library preparation, including: (a) nucleic acid extraction reagents (buffers, proteinase K, magnetic beads, or silica columns); (b) enzymes such as high-fidelity DNA polymerases (e.g., Platinum SuperFi II); (c) NGS adapters and index (barcode) sequences to allow multiplexing of multiple samples; (d) library purification beads (e.g., magnetic beads for size selection); or (e) sequencing buffers tailored for specific platforms (e.g., Illumina MiSeq); or any combination of (a)-(e)

The kit can further comprise a container for sample collection. Given the importance of the sample type, in some embodiments, the kit can be specifically tailored for the collection of uterine tissue. In some embodiments, the kit is specifically tailored for the collection of vaginal/cervical fluids. Exemplary containers include sterile tubes containing a DNA stabilization buffer (e.g., DNA/RNA Shield or Assay Assure) that preserves the microbiome profile during transport. For vaginal collection, the kit can comprise a synthetic or flocked swab and instructions for scrubbing the vaginal mucosa. For uterine collection, the kit can comprise a catheter or reagents compatible with tissue biopsies (e.g., lysis buffers).

In some embodiments, the kit comprises controls to ensure assay validity. Positive controls can comprise a mock microbial community (e.g., a mixture of Lactobacillus and Gardnerella DNA at known ratios) or synthetic DNA templates. Negative controls can comprise nuclease-free water or a blank sampling swab to detect environmental contamination.

### 6.3.2 Computational Components

In some embodiments, the kit comprises a non-transitory computer-readable medium which can store instructions that, when executed by a processor, perform the bioinformatic analysis. Exemplary non-transitory computer-readable media include, for example, a USB drive, a downloadable software package, or access to a cloud-based computing environment.

In some embodiments, the processor is instructed to first bioinformatically remove sequencing reads mapping to a human reference genome (e.g., hg38) to reduce noise from the host tissue. Following decontamination, the software quantifies the relative abundance of the specific panel of bacterial taxa disclosed herein.

The instructions can be configured to analyze the sequencing dataset using any of the methods disclosed herein. In some embodiments, the analysis is based on the subject's menstrual phase. If the sample is designated as proliferative, the software quantifies a panel comprising at least one, and preferably 2 to 17, of: Fenollaria, Anaeroglobus, Anaerococcus, Coprococcus, Prevotella, Varibaculum, Corynebacterium, Thalassobacillus, Staphylococcus, Priestia, Butyricimonas, Finegoldia, Mobiluncus, Cutibacterium, Peptoniphilus, Veillonella, and Gardnerella. In some embodiments, the software is programmed to identify these taxa based on sequences having at least 97% identity to SEQ ID NOs: 3-24. If the sample is designated as secretory, the software quantifies a panel comprising at least one, and preferably 2 to 10, of: Ureaplasma, Niallia, Murdochiella, Gardnerella, Lactobacillus, Lawsonella, Corynebacterium, Priestia, Finegoldia, and Dialister. In some embodiments, the software is programmed to identify these taxa based on sequences having at least 97% identity to SEQ ID NOs: 5, 16 and 24-35.

The computer-readable medium further stores instructions to calculate a Functional Dysbiosis Score (FDS), as described herein.

In some embodiments, the software comprises a stored, trained machine learning classifier (e.g., a Random Forest model). In some embodiments, this classifier has been previously trained using repeated random subsampling cross-validation on a dataset of confirmed endometriosis cases and controls, wherein the features were selected via MaAsLin2 analysis to control for Age and BMI. The processor inputs the quantified bacterial abundances and the calculated FDS into this frozen model to generate a classification output (e.g., a probability score or binary “Detected/Not Detected” result) indicating the likelihood of endometriosis.

In some embodiments, the kit can further comprise instructions. In some embodiments, the instructions are provided as a publication, a recording, a diagram, or a link to an online protocol. In some embodiments, the instructions can describe the method for collecting the sample, extracting DNA, performing the sequencing reaction, or utilizing the software to interpret the results, or any combination thereof.

### 6.4 METHODS OF TREATMENT

In some embodiments, upon assessment of a likelihood of endometriosis (e.g., a positive classification output generated by the machine learning classifier), the methods provided herein further comprise administering a treatment to the subject. By linking the accurate detection of the microbiome signature to a transformative therapeutic step, a complete clinical solution is provided. In some embodiments, methods provided herein comprise treating the subject assessed to have endometriosis with a therapy specifically selected to mitigate the disease pathology. In some embodiments, methods provided herein comprise providing a prophylactic treatment to a subject who is assessed to be predisposed to endometriosis based on an early-stage microbial signature, thereby potentially preventing lesion formation. In some embodiments, methods provided herein comprise providing an appropriate therapy for a subject having endometriosis that is assessed to be likely to progress into an advanced stage, as determined by the microbiome profile correlating with rASRM staging.

Treatment for endometriosis can involve medication, surgery, or microbiome modulation, depending on the severity of the symptoms, the specific microbial profile detected, and the goals of the treatment (e.g., pain relief versus the need for pregnancy).

In some embodiments, the treatment of endometriosis includes pain medication, such as nonsteroidal anti-inflammatory drugs (NSAIDs). Exemplary NSAIDs include ibuprofen (Advil, Motrin IB) or naproxen sodium (Aleve), which help ease painful menstrual cramps. However, because pain medication does not arrest the disease process, it is often combined with hormone therapies. Hormone therapies can slow endometrial tissue growth, prevent new implants of endometrial tissue, and minimize the inflammatory environment. Supplemental hormones suitable for use in the present methods include hormonal contraceptives, such as birth control pills, patches, and vaginal rings, which help control the hormones responsible for the buildup of endometrial tissue each month.

In some embodiments, the pharmaceutical intervention comprises the administration of Gonadotropin-Releasing Hormone (GnRH) modulators. This includes GnRH agonists (e.g., leuprolide, goserelin, nafarelin), which block the production of ovarian-stimulating hormones, lowering estrogen levels and preventing menstruation. This creates an artificial menopause that causes endometrial tissue to shrink. Alternatively, the treatment can comprise GnRH antagonists (e.g., elagolix, relugolix), which competitively bind to GnRH receptors to rapidly suppress pituitary gonadotropin production. The treatment can also comprise progestin therapy, utilizing a variety of delivery methods including an intrauterine device (IUD) with levonorgestrel (Mirena), a contraceptive implant, a contraceptive injection (depot medroxyprogesterone acetate), or a progestin pill (e.g., dienogest, norethindrone). These therapies can halt menstrual periods and the growth of endometrial implants. Furthermore, the treatment can comprise aromatase inhibitors, a class of medicines that reduce the amount of estrogen produced by the body. An aromatase inhibitor (e.g., letrozole, anastrozole) can be used in combination with a progestin or combination hormonal contraceptive to treat endometriosis.

In some embodiments, where pharmaceutical management is insufficient, or where the diagnostic assessment indicates advanced deep infiltrating endometriosis, the treatment can include surgeries. In some embodiments, the subject is treated with a conservative surgery to remove the endometriosis implants while preserving the uterus and ovaries, which is critical for patients wishing to conceive. The surgeries can be done laparoscopically (using the microbial assessment to guide the decision to operate) or, less commonly, through traditional abdominal surgery (laparotomy) in more extensive cases. The surgery can include removing the lesions (excising), destroying the lesions with intense heat (cauterizing or vaporizing), or removing the endometriosis patches.

In some embodiments, particularly for subjects who do not wish to bear children or who have severe, intractable disease, the treatment includes surgical removal of the uterus (hysterectomy). If the ovaries have endometriosis on them or if damage is severe, the treatment can also include removal of the ovaries and fallopian tubes along with the uterus (total hysterectomy and bilateral salpingo-oophorectomy). Additionally, if the subject presents with severe central abdominal pain, treatment can include surgery to sever pelvic nerves. Two procedures are typically used to sever different nerves in the pelvis: presacral neurectomy, which severs the nerves connected to the uterus, and Laparoscopic Uterine Nerve Ablation (LUNA), which severs nerves in the ligaments that secure the uterus.

A unique aspect of the present disclosure is the ability to tailor treatment to the specific dysbiosis detected. In some embodiments, treatment can involve attempting to restore the microbiome. For example, if the diagnostic panel detects a deficiency in protective lactobacilli, the treatment can comprise administering probiotics (e.g., Lactobacillus crispatus, Lactobacillus jensenii) either orally or vaginally. If the panel detects a high abundance of pathogenic taxa such as Gardnerella or Prevotella, the treatment may comprise administering targeted antibiotics (e.g., metronidazole, clindamycin) or prebiotics designed to selectively feed beneficial flora.

Since endometriosis can lead to trouble conceiving, and the methods herein can detect endometriosis in asymptomatic infertile women, the treatment can in some embodiments comprise fertility interventions. These range from stimulating the ovaries to produce more eggs (ovarian stimulation) to advanced reproductive technologies such as In Vitro Fertilization (IVF). The early detection of endometriosis via the microbiome signature allows for the implementation of these fertility treatments before extensive anatomical damage occurs.

### 6.5 EXEMPLARY EMBODIMENTS

Embodiment 1. A method for characterizing a microbiome to assess a likelihood of endometriosis in a subject, comprising: (a) obtaining a dataset representing a plurality of nucleic acid sequences derived from a sample obtained from the subject; (b) quantifying, from the dataset, a relative abundance of a panel of bacterial taxa; (c) calculating a Functional Dysbiosis Score (FDS) for the sample based on a relative abundance of Lactobacillus spp. and a cumulative relative abundance of a plurality of pathogenic taxa; and (d) processing the relative abundance of the panel of bacterial taxa and the FDS using a trained machine learning classifier to generate a classification output indicating the presence or absence of endometriosis.

Embodiment 2. The method of Embodiment 1, wherein the sample is obtained during the proliferative phase of a menstrual cycle.

Embodiment 3. The method of Embodiment 2, wherein the panel of bacterial taxa comprises at least one taxon selected from the group consisting of: Fenollaria, Anaeroglobus, Anaerococcus, Coprococcus, Prevotella, Varibaculum, Corynebacterium, Thalassobacillus, Staphylococcus, Priestia, Butyricimonas, Finegoldia, Mobiluncus, Cutibacterium, Peptoniphilus, Veillonella, and Gardnerella.

Embodiment 4. The method of Embodiment 3, wherein the panel comprises 2, 3, 4, 5, 6, 7, 8, 9, 10, 11, 12, 13, 14, 15, 16, or 17 of said bacterial taxa; optionally wherein the panel comprises at least one of Coprococcus and Butyricimonas; and at least one of Gardnerella and Prevotella.

Embodiment 5. The method of Embodiment 3, wherein the panel of bacterial taxa comprises: (i) at least one taxon selected from the group consisting of: Staphylococcus aureus, Fenollaria massiliensis, Priestia megaterium, Coprococcus catus, Butyricimonas faecihominis, Anaeroglobus geminatus, Anaerococcus octavius, Prevotella corporis, Varibaculum anthropi, Corynebacterium urealyticum, Thalassobacillus hwangdonensis, Corynebacterium tuberculostearicum, Staphylococcus intermedius, Finegoldia magna, Mobiluncus curtisii, Cutibacterium namnetense, Peptoniphilus harei, Priestia aryabhattai, Veillonella atypica, Prevotella timonensis, Prevotella bivia, and Gardnerella vaginalis; or (ii) at least one taxon selected from the group consisting of the taxa listed below, wherein each taxon is identified by the V4 region of a 16S rRNA gene sequence having at least 97% identity to the corresponding SEQ ID NO indicated in parentheses: (i) Staphylococcus sp.1 (SEQ ID NO:3); (ii) Fenollaria sp.1 (SEQ ID NO:4); (iii) Priestia sp.1 (SEQ ID NO:5); (iv) Coprococcus sp.1 (SEQ ID NO:6); (v) Butyricimonas sp.1 (SEQ ID NO: 7); (vi) Anaeroglobus sp.1 (SEQ ID NO:8); (vii) Anaerococcus sp.1 (SEQ ID NO:9); (viii) Prevotella sp.1 (SEQ ID NO: 10); (ix) Varibaculum sp.1 (SEQ ID NO:11); (x) Corynebacterium sp.1 (SEQ ID NO: 12); (xi) Thalassobacillus sp.1 (SEQ ID NO:13); (xii) Corynebacterium sp.2 (SEQ ID NO:14); (xiii) Staphylococcus sp.2 (SEQ ID NO:15); (xiv) Finegoldia sp.1 (SEQ ID NO:16); (xv) Mobiluncus sp.1 (SEQ ID NO:17); (xvi) Cutibacterium sp.1 (SEQ ID NO: 18); (xvii) Peptoniphilus sp.1 (SEQ ID NO:19); (xviii) Priestia sp.2 (SEQ ID NO:20); (xix) Veillonella sp.1 (SEQ ID NO:21); (xx) Prevotella sp.2 (SEQ ID NO:22); (xxi) Prevotella sp.3 (SEQ ID NO:23); and (xxii) Gardnerella sp.1 (SEQ ID NO:24).

Embodiment 6. The method of Embodiment 5, wherein the panel comprises 2, 3, 4, 5, 6, 7, 8, 9, 10, 11, 12, 13, 14, 15, 16, 17, 18, 19, 20, 21 or 22 of said bacterial taxa; optionally wherein the panel comprises (i) at least one of Coprococcus catus and Butyricimonas faecihominis; and at least one of Gardnerella vaginalis, Prevotella corporis, Prevotella timonensis, and Prevotella bivia; or (ii) at least one of Coprococcus sp.1 (SEQ ID NO:6) and Butyricimonas sp.1 (SEQ ID NO:7); and at least one of Gardnerella sp.1 (SEQ ID NO:24), Prevotella sp.1 (SEQ ID NO: 10), Prevotella sp.2 (SEQ ID NO:22), and Prevotella sp.3 (SEQ ID NO:23).

Embodiment 7. The method of any one of Embodiments 1 to 6, further comprising a step of measuring a serum progesterone level of the subject, wherein the proliferative phase is confirmed if the serum progesterone level is not above a reference level.

Embodiment 8. The method of Embodiment 7, wherein the reference level is 1.08 ng/mL.

Embodiment 9. The method of Embodiment 1, wherein the sample is obtained during the secretory phase of a menstrual cycle.

Embodiment 10. The method of Embodiment 9, wherein the panel of bacterial taxa comprises at least one taxon selected from the group consisting of: Ureaplasma, Niallia, Murdochiella, Gardnerella, Lactobacillus, Lawsonella, Corynebacterium, Priestia, Finegoldia, and Dialister.

Embodiment 11. The method of Embodiment 10, wherein the panel comprises 2, 3, 4, 5, 6, 7, 8, 9 or 10 of said bacterial taxa.

Embodiment 12. The method of Embodiment 10, wherein the panel of bacterial taxa comprises (i) at least one taxon selected from the group consisting of Ureaplasma urealyticum, Niallia oryzisoli, Murdochiella asaccharolytica, Gardnerella vaginalis, Lactobacillus iners, Lactobacillus jensenii, Lawsonella clevelandensis, Corynebacterium kroppenstedtii, Priestia megaterium, Lactobacillus crispatus, Finegoldia magna, Dialister hominis, Lactobacillus vaginalis, and Ureaplasma parvum; or (ii) at least one taxon selected from the group consisting of the taxa listed below, wherein each taxon is identified by the V4 region of a 16S rRNA gene sequence having at least 97% identity to the corresponding SEQ ID NO indicated in parentheses: Ureaplasma sp.1 (SEQ ID NO:25), Niallia sp.1 (SEQ ID NO:26), Murdochiella sp.1 (SEQ ID NO:27), Gardnerella sp.1 (SEQ ID NO:24), Lactobacillus sp.1 (SEQ ID NO:28), Lactobacillus sp.2 (SEQ ID NO:29), Lawsonella sp.1 (SEQ ID NO:30), Corynebacterium sp.3 (SEQ ID NO:31), Priestia sp.1 (SEQ ID NO:5), Lactobacillus sp.3 (SEQ ID NO:32), Finegoldia sp.1 (SEQ ID NO:16), Dialister sp.1 (SEQ ID NO:33), Lactobacillus sp.4 (SEQ ID NO:34), and Ureaplasma sp.2 (SEQ ID NO:35).

Embodiment 13. The method of Embodiment 12, wherein the panel comprises 2, 3, 4, 5, 6, 7, 8, 9, 10, 11, 12, 13, or 14 of said bacterial taxa.

Embodiment 14. The method of any one of Embodiments 9 to 13, further comprising a step of measuring a serum progesterone level of the subject, wherein the secretory phase is confirmed if the serum progesterone level is above a reference level.

Embodiment 15. The method of Embodiment 14, wherein the reference level is 1.08 ng/mL.

Embodiment 16. The method of any one of Embodiments 1 to 15, wherein the FDS is calculated by the formula: FDS=0.5×(1-ALacto)+10×Apatho, wherein ALacto is the relative abundance of Lactobacillus and Apatho is the cumulative relative abundance of the plurality of pathogenic taxa.

Embodiment 17. The method of any one of Embodiments 1 to 16, wherein the pathogenic taxa used to calculate the FDS comprises one or more taxa selected from: Gardnerella, Prevotella, Anaerococcus, Streptococcus, Megasphaera, Mobiluncus, Sneathia, Atopobium, Peptoniphilus, Mycoplasmoides, Ureaplasma, Bacteroides, Peptostreptococcus and Dialister.

Embodiment 18. The method of Embodiment 1 to 17, wherein the trained machine learning classifier is a Random Forest classifier.

Embodiment 19. The method of Embodiment 18, wherein the Random Forest classifier has been trained using repeated random subsampling cross-validation on a training dataset comprising microbiome profiles from subjects with confirmed endometriosis and controls.

Embodiment 20. The method of Embodiment 19, wherein the training data set is randomly split into 80% for training and 20% for testing in each iteration; optionally wherein the classifier is trained for at least 50 iterations of repeated cross-validation.

Embodiment 21. The method of Embodiment 19 or 20, wherein the bacterial taxa of the training dataset is selected by performing a multivariable association analysis.

Embodiment 22. The method of Embodiment 21, wherein the multivariable association analysis is performed using Microbiome Multivariable Associations with Linear Models (MaAsLin2), optionally controlled for a confounding variable; optionally wherein the confounding variables are age and Body Mass Index (BMI).

Embodiment 23. The method of any one of Embodiments 1 to 22, wherein obtaining the dataset comprises: (i) extracting genomic DNA from the sample; (ii) amplifying the V4 region of of bacterial 16S rRNA genes from the extracted genomic DNA to generate amplicons; and (iii) sequencing the amplicons.

Embodiment 24. The method of Embodiment 23, wherein the amplifying is performed using a primer set having the nucleotide sequences of SEQ ID NOs:1 and 2.

Embodiment 25. The method of Embodiment 23 or 24, further comprising bioinformatically removing sequencing reads mapping to a human reference genome prior to step (b).

Embodiment 26. The method of any one of Embodiments 1 to 25, wherein the sample comprises cervicovaginal fluid, vaginal mucus, cervical mucus, blood, vaginal mucosa, interstitial fluid, uterine fluid, cervical secretion, uterine tissue, reproductive cells, cervical cells, endometrial cells, fallopian cells, ovarian cells, or natural flora in a female reproductive tract.

Embodiment 27. The method of Embodiment 26, wherein the sample comprises endometrial cells.

Embodiment 28. The method of Embodiment 26, wherein the sample comprises vaginal mucus.

Embodiment 29. The method of Embodiment 26, wherein the sample comprises uterine tissue or uterine fluid.

Embodiment 30. The method of any one of Embodiments 1 to 29, comprising further measuring a protein biomarker or a miRNA biomarker for endometriosis in the sample.

Embodiment 31. The method of any one of Embodiments 1 to 30, wherein the subject has a clinical indicator for endometriosis, wherein the indicator is dysmenorrhea, lower abdominal pain, chronic pelvic pain, deep dyspareunia, dysuria, dyschezia, fatigue, or infertility, or any combination thereof.

Embodiment 32. The method of any one of Embodiments 1 to 30, wherein the subject is asymptomatic.

Embodiment 33. The method of any one of Embodiments 1 to 32, further comprising administering a treatment for endometriosis to the subject.

Embodiment 34. The method of Embodiment 33, wherein the treatment for endometriosis is pain medication, a hormone therapy, or a surgical procedure, or any combination thereof.

Embodiment 35. The method of Embodiment 33, wherein the treatment for endometriosis is laparoscopic excision, gonadotropin-releasing hormone (GnRH) agonist or antagonist, oral contraceptive, or progestin, or any combination thereof.

Embodiment 36. A kit for assessing whether a subject has endometriosis, comprising (1) a means for obtaining a dataset representing a plurality of nucleic acid sequences in a sample from the subject, and (2) a non-transitory computer-readable medium storing instructions that, when executed by a processor, cause the processor to: (i) receive the obtained dataset; (ii) quantify a relative abundance of a panel of bacterial taxa; (iii) calculate a FDS for the sample based on a relative abundance of Lactobacillus spp. and a cumulative relative abundance of a plurality of pathogenic taxa; and (iv) input the relative abundance of the panel of bacterial taxa and FDS into a trained machine learning classifier to generate a classification output indicating the presence or absence of endometriosis.

Embodiment 37. The kit of Embodiment 36, wherein the sample is obtained during the proliferative phase of a menstrual cycle.

Embodiment 38. The kit of Embodiment 37, wherein the panel of bacterial taxa comprises at least one taxon selected from the group consisting of: Fenollaria, Anaeroglobus, Anaerococcus, Coprococcus, Prevotella, Varibaculum, Corynebacterium, Thalassobacillus, Staphylococcus, Priestia, Butyricimonas, Finegoldia, Mobiluncus, Cutibacterium, Peptoniphilus, Veillonella, and Gardnerella.

Embodiment 39. The kit of Embodiment 38, wherein the panel comprises 2, 3, 4, 5, 6, 7, 8, 9, 10, 11, 12, 13, 14, 15, 16, or 17 of said bacterial taxa; optionally wherein the panel comprises at least one of Coprococcus and Butyricimonas; and at least one of Gardnerella and Prevotella.

Embodiment 40. The kit of Embodiment 38, wherein the panel of bacterial taxa comprises (i) at least one taxon selected from the group consisting of: Staphylococcus aureus, Fenollaria massiliensis, Priestia megaterium, Coprococcus catus, Butyricimonas faecihominis, Anaeroglobus geminatus, Anaerococcus octavius, Prevotella corporis, Varibaculum anthropi, Corynebacterium urealyticum, Thalassobacillus hwangdonensis, Corynebacterium tuberculostearicum, Staphylococcus intermedius, Finegoldia magna, Mobiluncus curtisii, Cutibacterium namnetense, Peptoniphilus harei, Priestia aryabhattai, Veillonella atypica, Prevotella timonensis, Prevotella bivia, and Gardnerella vaginalis; or (ii) at least one taxon selected from the group consisting of the taxa listed below, wherein each taxon is identified by the V4 region of a 16S rRNA gene sequence having at least 97% identity to the corresponding SEQ ID NO indicated in parentheses: (i) Staphylococcus sp.1 (SEQ ID NO:3); (ii) Fenollaria sp.1 (SEQ ID NO:4); (iii) Priestia sp.1 (SEQ ID NO:5); (iv) Coprococcus sp.1 (SEQ ID NO:6); (v) Butyricimonas sp.1 (SEQ ID NO:7); (vi) Anaeroglobus sp.1 (SEQ ID NO:8); (vii) Anaerococcus sp.1 (SEQ ID NO:9); (viii) Prevotella sp.1 (SEQ ID NO: 10); (ix) Varibaculum sp.1 (SEQ ID NO:11); (x) Corynebacterium sp.1 (SEQ ID NO: 12); (xi) Thalassobacillus sp.1 (SEQ ID NO:13); (xii) Corynebacterium sp.2 (SEQ ID NO:14); (xiii) Staphylococcus sp.2 (SEQ ID NO: 15); (xiv) Finegoldia sp.1 (SEQ ID NO: 16); (xv) Mobiluncus sp.1 (SEQ ID NO: 17); (xvi) Cutibacterium sp.1 (SEQ ID NO:18); (xvii) Peptoniphilus sp.1 (SEQ ID NO:19); (xviii) Priestia sp.2 (SEQ ID NO:20); (xix) Veillonella sp.1 (SEQ ID NO:21); (xx) Prevotella sp.2 (SEQ ID NO:22); (xxi) Prevotella sp.3 (SEQ ID NO:23); and (xxii) Gardnerella sp.1 (SEQ ID NO:24).

Embodiment 41. The kit of Embodiment 40, wherein the panel comprises 2, 3, 4, 5, 6, 7, 8, 9, 10, 11, 12, 13, 14, 15, 16, 17, 18, 19, 20, 21 or 22 of said bacterial taxa; optionally wherein the panel comprises (i) at least one of Coprococcus catus and Butyricimonasfaecihominis; and at least one of Gardnerella vaginalis, Prevotella corporis, Prevotella timonensis, and Prevotella bivia; or (ii) at least one of Coprococcus sp.1 (SEQ ID NO:6) and Butyricimonas sp.1 (SEQ ID NO:7); and at least one of Gardnerella sp.1 (SEQ ID NO:24), Prevotella sp.1 (SEQ ID NO: 10), Prevotella sp.2 (SEQ ID NO:22), and Prevotella sp.3 (SEQ ID NO:23).

Embodiment 42. The kit of Embodiment 36, wherein the sample is obtained during the secretory phase of a menstrual cycle.

Embodiment 43. The kit of Embodiment 42, wherein the panel of bacterial taxa comprises at least one taxon selected from the group consisting of: Ureaplasma, Niallia, Murdochiella, Gardnerella, Lactobacillus, Lawsonella, Corynebacterium, Priestia, Finegoldia, and Dialister.

Embodiment 44. The kit of Embodiment 43, wherein the panel comprises 2, 3, 4, 5, 6, 7, 8, 9 or 10 of said bacterial taxa.

Embodiment 45. The kit of Embodiment 43, wherein the panel of bacterial taxa comprises (i) at least one taxon selected from the group consisting of Ureaplasma urealyticum, Niallia oryzisoli, Murdochiella asaccharolytica, Gardnerella vaginalis, Lactobacillus iners, Lactobacillus jensenii, Lawsonella clevelandensis, Corynebacterium kroppenstedtii, Priestia megaterium, Lactobacillus crispatus, Finegoldia magna, Dialister hominis, Lactobacillus vaginalis, and Ureaplasma parvum; or (ii) at least one taxon selected from the group consisting of the taxa listed below, wherein each taxon is identified by the V4 region of a 16S rRNA gene sequence having at least 97% identity to the corresponding SEQ ID NO indicated in parentheses: Ureaplasma sp.1 (SEQ ID NO:25), Niallia sp.1 (SEQ ID NO:26), Murdochiella sp.1 (SEQ ID NO:27), Gardnerella sp.1 (SEQ ID NO:24), Lactobacillus sp.1 (SEQ ID NO:28), Lactobacillus sp.2 (SEQ ID NO:29), Lawsonella sp.1 (SEQ ID NO:30), Corynebacterium sp.3 (SEQ ID NO:31), Priestia sp.1 (SEQ ID NO:5), Lactobacillus sp.3 (SEQ ID NO:32), Finegoldia sp.1 (SEQ ID NO:16), Dialister sp.1 (SEQ ID NO:33), Lactobacillus sp.4 (SEQ ID NO:34), and Ureaplasma sp.2 (SEQ ID NO:35).

Embodiment 46. The kit of Embodiment 45, wherein the panel comprises 2, 3, 4, 5, 6, 7, 8, 9, 10, 11, 12, 13, or 14 of said bacterial taxa.

Embodiment 47. The kit of any one of Embodiments 36 to 46, wherein the FDS is calculated by the formula: FDS=0.5×(1-ALacto)+10×Apatho, wherein ALacto is the relative abundance of Lactobacillus and Apatho is the cumulative relative abundance of the plurality of pathogenic taxa.

Embodiment 48. The kit of any of Embodiments 36 to 47, wherein the pathogenic taxa used to calculate the FDS comprises one or more genera selected from: Gardnerella, Prevotella, Anaerococcus, Streptococcus, Megasphaera, Mobiluncus, Sneathia, Atopobium, Peptoniphilus, Mycoplasmoides, Ureaplasma, Bacteroides, Peptostreptococcus and Dialister.

Embodiment 49. The kit of Embodiments 36 to 48, wherein the trained machine learning classifier is a Random Forest classifier.

Embodiment 50. The kit of Embodiment 49, wherein the Random Forest classifier has been trained using repeated random subsampling cross-validation on a training dataset comprising microbiome profiles from subjects with confirmed endometriosis and controls.

Embodiment 51. The kit of Embodiment 50, wherein the data is randomly split into 80% for training and 20% for testing in each iteration; optionally wherein the classifier is trained over 50 iterations of repeated cross-validation.

Embodiment 52. The kit of Embodiment 50 or 51, wherein the bacterial taxa of the training dataset is selected by performing a multivariable association analysis.

Embodiment 53. The kit of Embodiment 52, wherein the multivariable association analysis is performed using MaAsLin2, optionally controlled for a confounding variable; optionally wherein the confounding variables are age and BMI.

Embodiment 54. The kit of any one of Embodiments 36 to 53, wherein the means for obtaining a dataset comprises a primer set configured to amplify the V4 region of bacterial 16S rRNA; optionally wherein the primers have nucleotide sequences of SEQ ID NOs:1 and 2.

Embodiment 55. The kit of any one of Embodiments 36 to 54, wherein the processor is to bioinformatically remove sequencing reads mapping to a human reference genome prior to step (ii).

Embodiment 56. The kit of any one of Embodiments 36 to 55, wherein the sample comprises cervicovaginal fluid, vaginal mucus, cervical mucus, blood, vaginal mucosa, interstitial fluid, cervical secretion, uterine tissue, reproductive cells, cervical cells, endometrial cells, fallopian cells, ovarian cells, or natural flora in a female reproductive tract.

Embodiment 57. The kit of Embodiment 56, wherein the sample comprises endometrial cells.

Embodiment 58. The kit of Embodiment 56, wherein the sample comprises vaginal mucus.

Embodiment 59. The kit of Embodiment 56, wherein the sample comprises uterine tissue or uterine fluid.

Embodiment 60. The kit of any one of Embodiments 36 to 59, wherein the kit further comprises a container for sample collection.

### 6.6 EXPERIMENTAL

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

### 6.6.1 Uterine Microbiome Molecular Analysis to Screen for Endometriosis

Introduction: The clinical presentation of endometriosis varies widely, from asymptomatic to severe with often infertility also presented. Screening methods today include imaging which is subject to operator expertise and poor specificity. Here we demonstrate efficacy in screening for endometriosis by using bioinformatics to assess uterine biome composition among women about to undergo surgery for suspected endometriosis.

Methods: Under IRB approval, uterine tissue biopsies were obtained from (n=98) women prior to undergoing laparoscopy surgery with histology for suspected endometriosis. The endometrial microbiome profile was determined based on barcoded sequencing of the bacterial 16S rRNA gene V4 region. Bioinformatic/statistical analysis to identify and quantify the composition of the abnormal and normal bacteria was performed.

Results: Of the 98 cases, 54 cases were histologically confirmed to be positive for endometriosis. Molecular analysis of the microbiome revealed that 35 of the endometriosis confirmed cases (65%) had an abnormal microbiome composition. When comparing early to late stage endometriosis, 7 of 10 (70%) and 25 of 44 (57%) cases, respectively, displayed abnormal microbiomes with a higher prevalence of Gardnerella and Streptococcus. In contrast, of the 44 endometriosis negative cases, microbiome composition was abnormal in 15 (34%) cases.

### Conclusions

These data indicate that uterine microbiome analysis can serve as a screen test to identify women at risk for endometriosis, potentially enabling earlier detection and intervention.

### 6.6.2 Microbiome Markers for Risk Assessment and Staging of Endometriosis: A Comparative Analysis of the Secretory and Proliferative Phases

This study was designed to reveal the relationship between the vaginal microbiome and the risk and progression of endometriosis. By analyzing microbiome data from women in both the secretory and proliferative phases of the menstrual cycle, specific bacterial markers indicative of early risk for endometriosis as well as capable of differentiating between early-stage and late-stage disease were identified.

### Study Design and Sample Groups

The study samples were drawn from three distinct groups: Group 1 (Patient Control) includes symptomatic individuals who did not have endometriosis, Group 2 (Early-stage Disease) includes individuals with early-stage endometriosis, and Group 3 (Late-stage Disease) includes those diagnosed with late-stage endometriosis. Samples were collected from participants during two phases of the menstrual cycle: the secretory phase and the proliferative phase. These samples were subjected to microbiome profiling.

### Findings:

As shown in Table 1 below, in the secretory phase, several bacterial markers were identified that distinguished late-stage endometriosis from both healthy controls and early-stage patients. Specifically, the presence of Streptococcus, Escherichia, Staphylococcus, Bacteroides, Anaerococcus, Haemophilus, Veillonella, Dialister, and Finegoldia was strongly associated with late-stage endometriosis (Group 3). In contrast, while Gardnerella was present across all groups, its combination with Atopobium was a distinctive marker for early-stage disease (Group 2). AP: abnormal positive; AN: abnormal negative

Additionally, as shown in Table 2 below, during the proliferative phase, Megasphaera, Escherichia, Enterococcus, Flavobacterium, and Sneathia were found prevalent in both early and late-stage endometriosis (Group 2 and Group 3). Similar to the secretory phase, the combination of Gardnerella and Atopobium was observed in both early and late-stage disease, highlighting its potential as a consistent marker for endometriosis risk. Additionally, certain bacteria, such as Escherichia, were noted to be present in both phases and across different disease stages, indicating a possible broad role in the microbial dysbiosis associated with endometriosis.

6.6.3 Uterine Microbiome Signatures Associated with Endometriosis

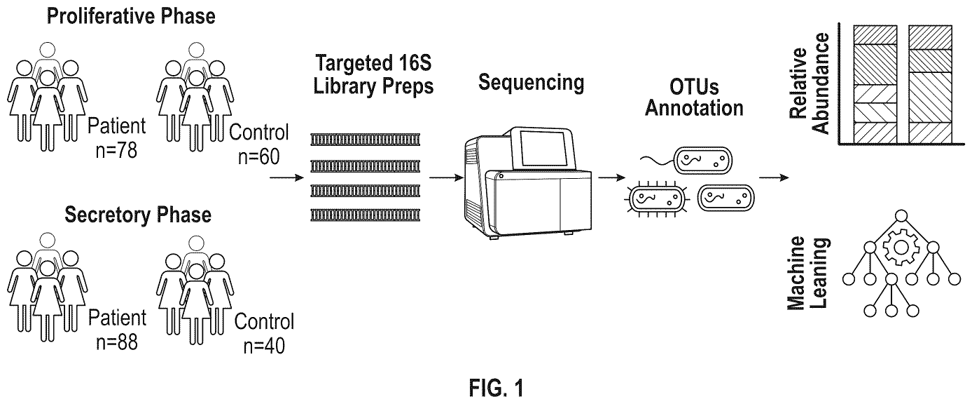

Overview: To examine microbiome features associated with endometriosis in a phase-dependent manner, we analyzed uterine microbiomes in 266 samples from women in either the proliferative or secretory phases. A total of 138 uterine tissue samples were collected from women in the proliferative phase of their menstrual cycle. Among these, 78 samples were obtained from women with a laparoscopic diagnosis of endometriosis, while the remaining 60 were from women without the disease. An additional 128 uterine tissue samples were collected during the secretory phase, including 88 from women diagnosed with endometriosis and 40 from unaffected individuals (Table 3; FIG. 1). Total genomic DNA was extracted from all tissue samples and used to prepare targeted bacterial 16S rRNA gene libraries for sequencing, as detailed below. Raw sequence data were processed to remove technical artifacts, host DNA, and environmental contaminants. High-confidence bacterial reads were then taxonomically annotated using an internally curated version of the Greengenes2 database. Downstream analyses focused on identifying differentially abundant microbial taxa associated with endometriosis and determining taxa with potential predictive value for disease diagnosis.

### Materials and Methods:

Specimen collection: Endometrial tissue samples were collected from 266 individuals, all of whom were clinically suspected of having a gynecologic condition and scheduled for laparoscopy with histopathological evaluation. To explore menstrual cycle-related differences, samples were obtained from women in either the proliferative or secretory phases of their cycle. The menstrual phase was initially assessed by physicians or surgeons based on self-reported cycle days and clinical evaluations. To confirm this classification, serum progesterone levels were measured using a protein assay from Kangrun Biotech Co. Ltd. (Guangdong, China), with levels above 1.08 ng/mL indicating the secretory phase, as per the manufacturer's guidelines. Among the samples, 138 were from the proliferative phase (78 from individuals with endometriosis and 60 from controls), and 128 were from the secretory phase (88 with endometriosis and 40 controls). Endometriosis was diagnosed and confirmed via gold-standard laparoscopic surgery. All participants provided written informed consent. The collected tissue samples were immediately transported at 4° C. to the Heranova Lifesciences laboratory and stored at −20° C. upon arrival.

Uterine tissue processing and targeted 16S library preparations: A 3-5 mm fragment of endometrial tissue from each sample was placed into an individual centrifuge tube with 20 μL of Proteinase K and 180 μL of Buffer ATL. The mixture was vortexed thoroughly and incubated at 58° C. with shaking at 1200 rpm for 3 hours. After incubation, each sample was mixed with 210 μL of Buffer ATL and homogenized using the TissueLyser II (2 minutes at 30 Hz, 1-minute pause, repeated for 15 cycles), and DNA was extracted using the QIAsymphony SP instrument (QIAGEN, 35459). DNA concentration and purity were assessed using the MultiSkan GO spectrophotometer (Thermo, 1510). The V4 variable region of the 16S rRNA was amplified using Invitrogen Platinum SuperFi II DNA Polymerase with the following PCR conditions: initial denaturation at 98° C. for 30 seconds (1 cycle), followed by 30 cycles of 98° C. for 10 seconds, 60° C. for 10 seconds, and 72° C. for 30 seconds, and a final extension at 72° C. for 5 minutes before holding at 4° C. The forward primer is 5′-TAATTGTGTGCCAGCmGCCGCGGTAA-3′ (SEQ ID NO: 1) while the reverse primer is 5′-TCAGCCGGACTAChvGGGTwTCTAAT-3′ (SEQ ID NO:2). The PCR products were purified using VAHTS DNA Clean Beads. Adapter ligation was carried out using the UltraClean Universal DNA Library Prep Kit for Illumina V3 (Vazyme, UND607-02). First, 45 μL of End Repair reaction mix was added to the purified PCR product, followed by incubation at 20° C. for 15 minutes, 65° C. for 15 minutes, and held at 4° C. The ligation reaction mix was prepared on ice, added to the end-repaired DNA, and incubated at 20° C. for 15 minutes, then held at 4° C. The ligated products were purified again with VAHTS DNA Clean Beads. Library amplification was performed under the following thermal conditions: 95° C. for 3 minutes (1 cycle), then 5 cycles of 98° C. for 20 seconds, 60° C. for 15 seconds, and 72° C. for 30 seconds, with a final extension at 72° C. for 5 minutes and a hold at 4° C. The final libraries were purified using VAHTS DNA Clean Beads. Library concentrations were quantified using the KAPA Library Quantification Kit (KAPA, KK4824), and fragment sizes were evaluated with the Agilent 4200 TapeStation (Agilent, G2991A). Sequencing was performed on the Illumina MiSeq platform using the MiSeq Reagent Kit v2 (300 cycles).

Bioinformatic processing of targeted 16S sequencing data: The demultiplexed FASTQ files from Illumina MiSeq sequencing were processed to extract the forward reads. To improve data quality, a two-step trimming and filtering process was employed. First, fastp (Chen et al., Bioinformatics. 2018; 34(17):i884-i890) was used to identify and remove polyX artifacts-artificial stretches of a single nucleotide—commonly introduced during sequencing. Next, cutadapt (Marcel, EMBnet.journal. 2011; 17(1):10-12) was used to trim any residual adapter sequences from the reads. To eliminate host-derived contamination, the filtered reads were aligned to the human reference genome (hg38) using Bowtie2 (Langmead & Salzberg, Nature Methods. 2012; 9:357-359). Alignment results were processed with SAMtools (Li et al., Bioinformatics. 2009; 25(16):2078-2079), and reads mapping to the human genome were removed, ensuring that only non-host (primarily bacterial) sequences were retained for microbiome analysis.

The resulting high-quality, non-human reads were then imported into the QIIME2 platform (Bolyen et al. Nature Biotechnology. 2019; 37:852-857) for microbial community analysis. Within QIIME2, chimeric sequences were identified and removed using the vsearch uchime-denovo method. Subsequently, redundant sequences were collapsed using vsearch dereplicate-sequences, enhancing computational efficiency and reducing noise. The remaining high-confidence bacterial reads were annotated using an internally curated version of the Greengenes2 reference database (McDonald et al. Nature Biotechnology. 2024; 42:715-718). Taxonomic assignments were made using the Greengenes2 taxonomy-from-table classifier, providing genus-level and species-level annotations where possible. To ensure the validity and accuracy of the microbiome profiles, decontamination was performed using SCRuB (Austin et al., Nature Biotechnology, 2023; 41:1820-1828), a statistical tool designed to identify and remove background contaminants. A blank negative control, which underwent the entire experimental workflow alongside the tissue samples, was included in the analysis to model and subtract any environmental or reagent-based contaminants. This approach ensured that the final dataset reflected true biological signals and minimized the risk of false microbial detection. Microbiome Shannon index and beta diversity were calculated and visualized using vegan (Vegan, Science, 2003; 14(6):927-930) in R (v.4.4.2). A functional dysbiosis score was computed for each sample using the following formula (0.5*(1-Lactobacillus)+10*(Pathogenic taxa)) where pathogenic taxa consisted of genus commonly associated bacterial vaginosis including Gardnerella, Prevotella, Anaerococcus, Streptococcus, Megasphaera, Mobiluncus, Sneathia, Atopobium, Peptoniphilus, Mycoplasmoides, Ureaplasma, Bacteroides, Peptostreptococcus and Dialister.

Disease prediction model construction using a random forest classifier: Samples were divided into proliferative (n=138) and secretory (n=128) groups for the following analysis, and the subsequent analysis was based on bacterial species relative abundances. MaAsLin2 (Mallick et al., PLoS Computational Biology, 2021; 17(11):e100944) was performed to determine the multivariable association between bacterial species and endometriosis/non-endometriosis groups (P≤0.05), with age and BMI controlled using R package MaAsLin2. Features with importance scores ≥0.015 to the endometriosis/non-endometriosis groups among bacterial species were selected via random forest implemented in Python sklearn package. Models to predict endometriosis/non-endometriosis were built based on features that were selected by MaAsLin2, by random forest feature scoring and by the addition of the functional dysbiosis score. Model performance was assessed through 50 iterations of repeated random subsampling cross-validation, in which the data was randomly split into 80% for training and 20% for testing in each iteration. This strategy helped account for variability arising from random data splits and yielded a more robust estimate of the predictive accuracy.

### Results:

### Uterine Microbiome Landscape in the Study Cohort

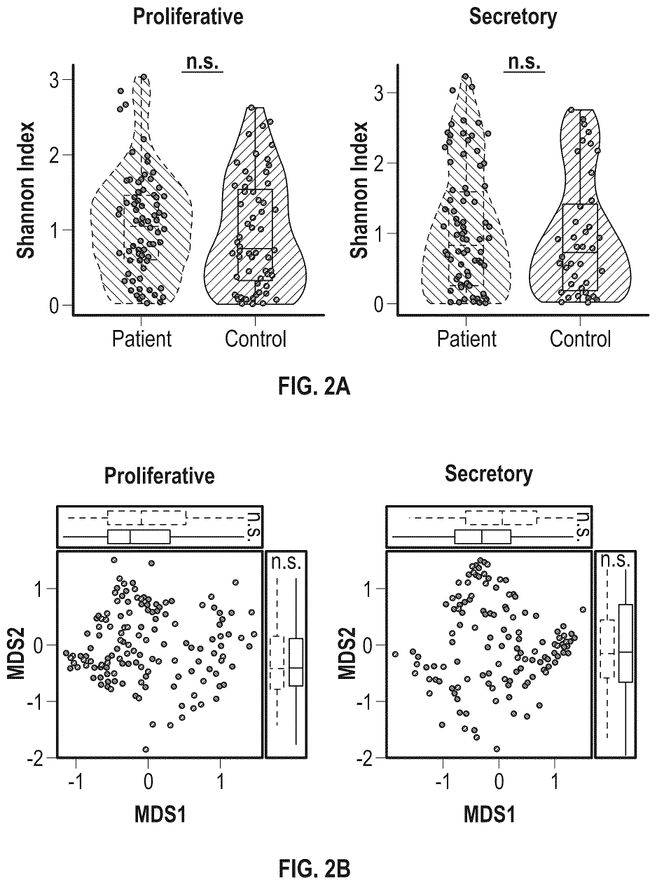

The initial objective of the study was to evaluate both alpha and beta diversity of the uterine microbiome in women diagnosed with endometriosis compared to those without the disease, stratified by the proliferative and secretory phases of the menstrual cycle. Alpha diversity, which reflects the richness and evenness of microbial species within individual samples, was assessed using the Shannon Index. No statistically significant differences in alpha diversity were observed between endometriosis and control groups in either the proliferative or secretory phase (FIG. 2A). Similarly, beta diversity, which measures compositional differences in microbial communities between groups, was evaluated using the Bray-Curtis dissimilarity metric. This analysis also revealed no significant differences between women with and without endometriosis across both menstrual phases (FIG. 2B). These findings suggest that the overall diversity, including both the number of microbial taxa and their relative abundance distribution, is comparable between affected and unaffected individuals, irrespective of the menstrual cycle phase.

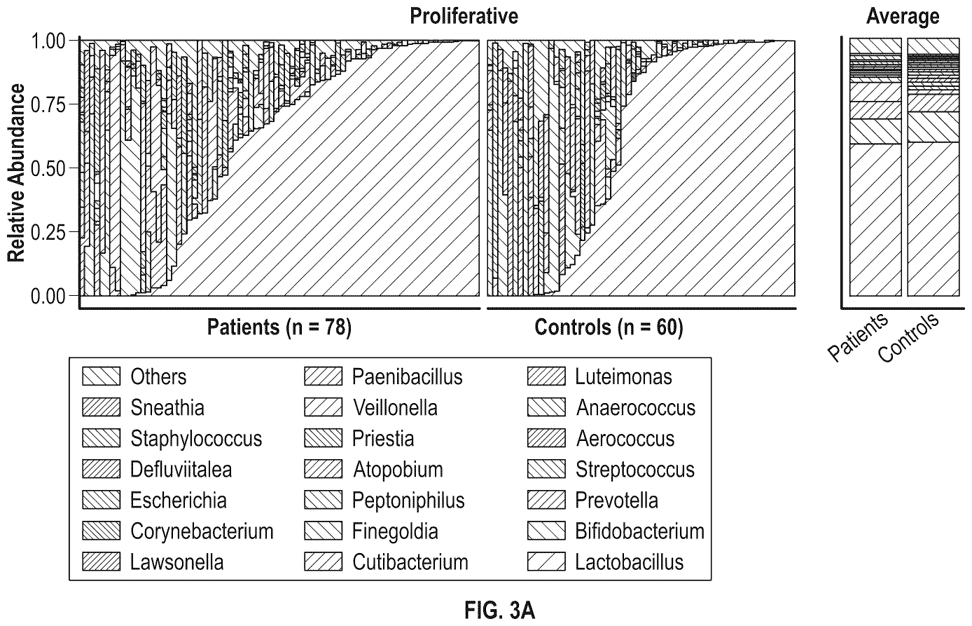

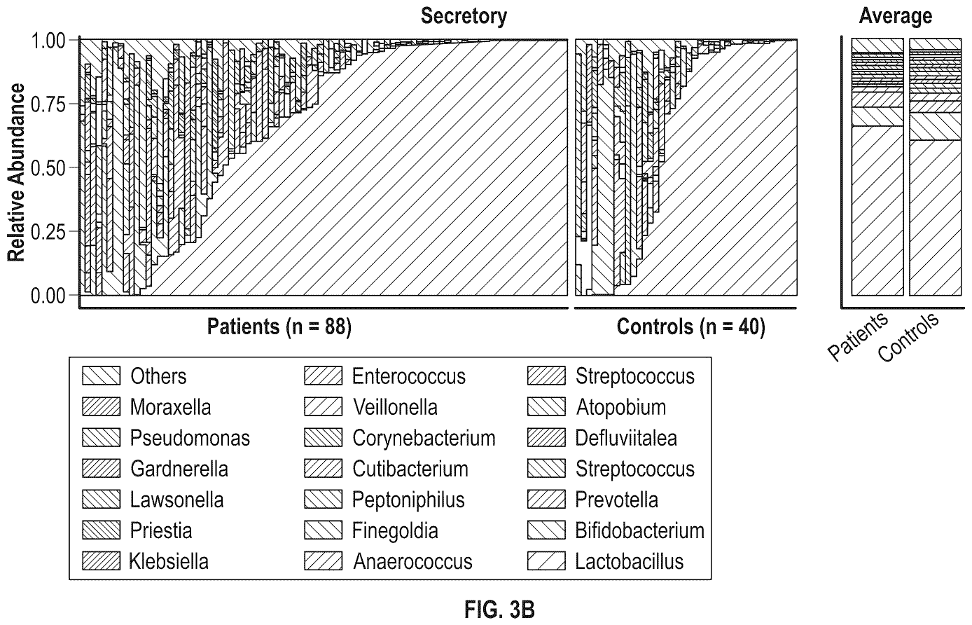

Genus-level analysis revealed substantial variability in the relative abundance of Lactobacillus among individuals, both in patients and controls, across both menstrual phases. Although Lactobacillus is typically considered a hallmark of a healthy vaginal microbiome, its levels varied considerably, especially among patients with endometriosis in the proliferative phase compared to the controls (FIGS. 3A-3B). An analysis of the top 20 most abundant genera in both proliferative and secretory phase samples showed that the overall distribution of relative abundance was similar between patients and controls, with most of these dominant taxa not differentially abundant. One notable observation was made: the genus Prevotella showed a trend toward enrichment in patient samples from the proliferative phase (p=0.0509). Bacteria species in the genus Prevotella are commonly associated with vaginal dysbiosis and pro-inflammatory states (Ding et al., J Gynecol Obstet Hum Reprod. 2021; 50(9):102174).

### Differentially Abundant and Machine-Learning Informative Taxa

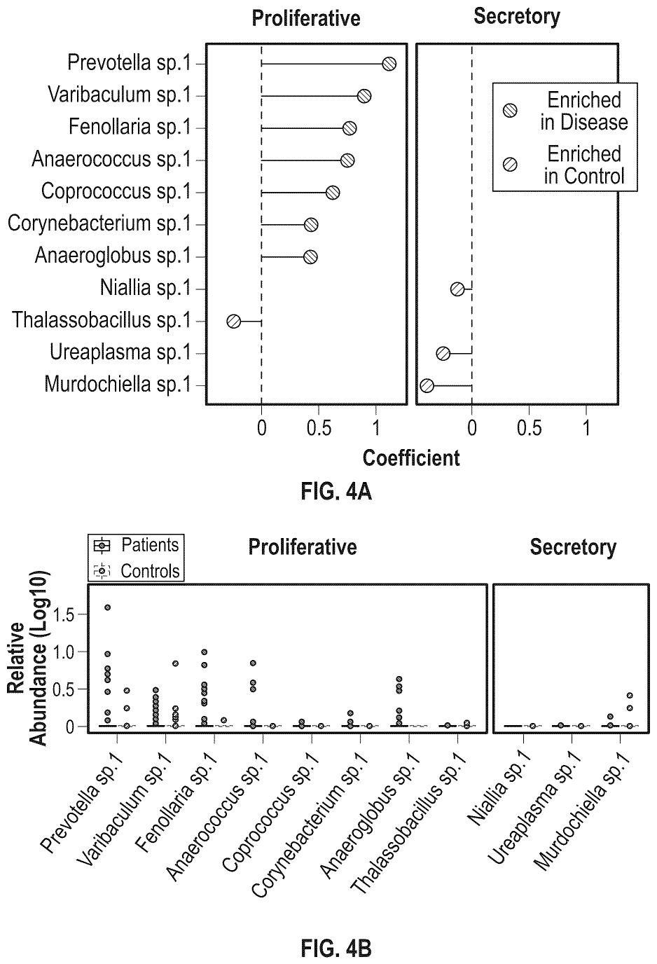

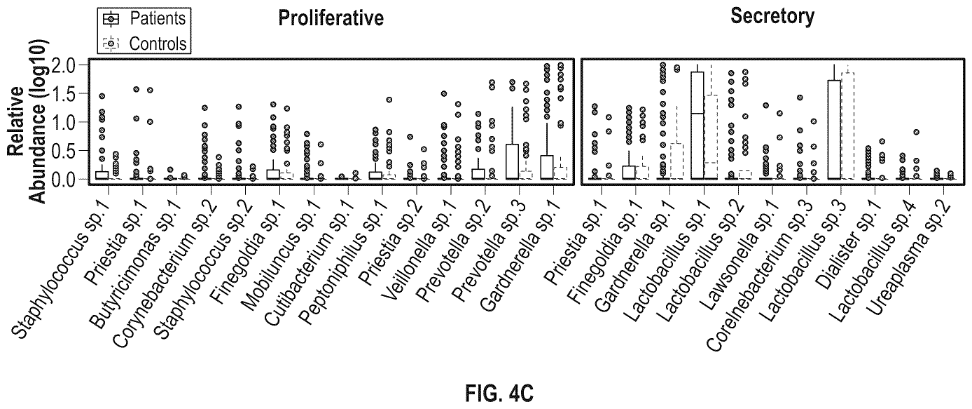

A more in-depth analysis of sub-genus level taxonomic units revealed eight taxa that were differentially abundant (p≤0.05) between patients and controls in the proliferative phase, and three differential taxa in the secretory phase (FIG. 4A), after adjusting for potential confounding effects of BMI and age. After corrected for multiple comparisons using false discovery rate (FDR) adjustment, however, the initially observed differential taxa no longer reached statistical significance (i.e., FDR >0.05). Nonetheless, subtle yet consistent shifts in microbial composition across multiple taxa could carry predictive value (Chang et al., Nature Communications, 2024; 15:7447). Therefore, we employed machine learning approaches to integrate these signals, under the premise that the cumulative effect of multiple informative taxa would enable and/or enhance predictive performance in distinguishing disease states (Wang et al., Frontiers in Cellular and Infection Microbiology. 2025; 15:1582522).

To develop the feature set for supervised machine learning classification, we implemented a three-step selection strategy combining statistical and algorithmic criteria. First, we identified differential taxa (i.e., nominal p-values ≤0.05; FIGS. 4A & 4B), indicating potential biological relevance despite not meeting strict multiple-testing thresholds. Inclusion of these taxa was to ensure that subtle, non-random differences were not overlooked. In the second step, we applied a machine learning-based feature selection process by systematically evaluating the importance of each taxon detected in our profiling pipeline. This involved training preliminary models to score each taxon's contribution to classification performance, using a predefined threshold of feature importance score ≥0.015 as a cutoff for inclusion. Taxa meeting this threshold were selected as additional candidates for the final feature set (FIG. 4C; Table 4). A subset of taxa was identified exclusively through the feature importance criterion. Specifically, 14 additional taxa including two other Prevotella spp. were added in the proliferative phase. In the secretory phase, 11 additional taxa were incorporated. Notably, Gardnerella sp.1, a taxon traditionally linked to bacterial vaginosis was included in both proliferative and secretory cohorts. In the third step, a functional dysbiosis score (FDS) was calculated for each sample (Table 5), representing an aggregate measure of microbial dysbiosis within the uterine tissue. The final feature set used for model training therefore comprised three components: the differential taxa, taxa selected by feature importance scoring, and the FDS (Tables 6-7).

### Diagnostic Performance of Machine-Learning Models in Endometriosis Detection

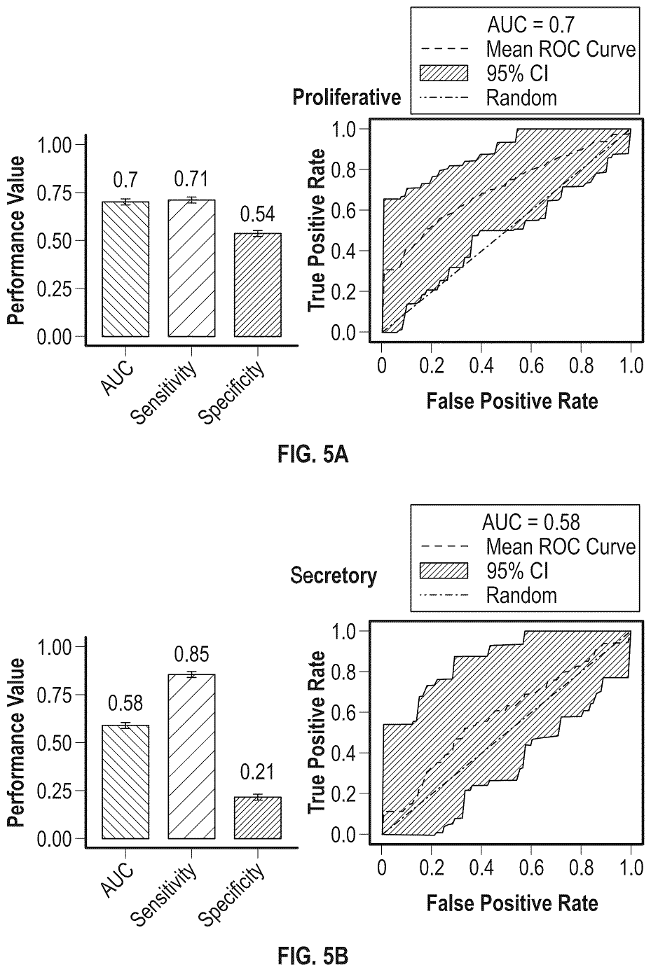

Using the microbial profiles from the proliferative phase, we achieved promising predictive performance in distinguishing endometriosis patients from controls. Specifically, across 50 rounds of repeated random subsampling cross-validation, the average AUC reached 0.70, indicating reasonable discriminative capability. The model demonstrated a sensitivity of 0.71, and a specificity of 0.54 (FIG. 5A). The overall performance indicates that the microbiome during the proliferative phase carries meaningful signals that could aid in endometriosis diagnosis. Additionally, models trained on the microbial profiles from secretory phase showed an average AUC of 0.58 (FIG. 5B). Overall, these findings demonstrate that microbial signatures differ in both menstrual phases, and that the proliferative phase carries relatively more informative profiles. From a clinical and biological perspective, these results underscore the importance of menstrual cycle timing when considering the microbiome as a diagnostic aid for endometriosis. The result also indicates that cycle-phase-specific sampling can help optimize microbiome-based diagnostics, and that models can be further optimized by integrating hormonal phase information.

Conclusion: This study investigated the uterine microbiome in women with and without endometriosis, with a focus on menstrual cycle phase-specific microbial signatures and their predictive value for disease status. The differentially abundant taxa, coupled with the use of supervised machine-learning allowed us to uncover patterns of microbial variation that hold diagnostic potential.

TABLE 1
Microbiome analysis - secretory phase

| Sample | | | Lacto- | | | | | | | |
|---|---|---|---|---|---|---|---|---|---|---|
| ID | Group | Result | bacillus | Gardnerella | Streptococcus | Prevotella | Klebsiella | Escherichia | Atopobium | Enterobacter |
| 1 | 1 | AP | | 26% | | | | | | |
| 2 | 1 | AP | 13% | | | | 7% | | | 9% |
| 3 | 1 | AP | | 38% | | | | | | |
| 4 | 1 | AP | | 67% | | 11% | | | | |
| 5 | 2 | AN | 68% | | | | | | | |
| 6 | 3 | AP | | 70% | | | | | 25% | |
| 7 | 3 | AP | 62% | | 34% | | | | | |
| 8 | 3 | AP | 45% | | 12% | 6% | | | | |
| 9 | 3 | AN | 82% | | | 8% | | | | |
| 10 | 3 | AP | | 6% | | | | | | |
| 11 | 3 | AP | 18% | | | 6% | | 12% | | |
| 12 | 3 | AP | | | | | 9% | 22% | | |
| 13 | 3 | AP | | | 54% | | | | | |
| 14 | 3 | AP | | | 6% | 9% | | | | |
| 15 | 3 | AP | | | 43% | | | | | |
| Sample | | | Staphy- | | | | | | | |
| ID | Group | Result | lococcus | Bacteroides | Anaerococcus | Haemophilus | Corynebacterium | Veillonella | Dialister | Finegoldia |
| 1 | 1 | AP | | | | | | | | |
| 2 | 1 | AP | | | | | | | | |
| 3 | 1 | AP | | | | | | | | |
| 4 | 1 | AP | | | | | | | | |
| 5 | 2 | AN | | | | | 14% | | | |
| 6 | 3 | AP | | | | | | | | |
| 7 | 3 | AP | | | | | | | | |
| 8 | 3 | AP | 7% | | | 8% | 6% | | | |
| 9 | 3 | AN | | | | | | | | |
| 10 | 3 | AP | | 15% | 6% | | | | | |
| 11 | 3 | AP | | 15% | | | | | | |
| 12 | 3 | AP | | | | 10% | | 10% | | |
| 13 | 3 | AP | | | | | | | | |
| 14 | 3 | AP | | | 9% | | | | 9% | 7% |
| 15 | 3 | AP | | | | | | | | |

TABLE 2
Microbiome analysis - proliferative phase

| Sample ID | Group | Result | Gardnerella | Prevotella | Streptococcus | Atopobium | Dialister | Enterobacter | Megasphaera |
|---|---|---|---|---|---|---|---|---|---|
| 1 | 1 | AN | | 10% | | | | | |
| 2 | 1 | AN | | 9% | | | 6% | | |
| 3 | 1 | AN | | | | | | | |
| 4 | 1 | AP | 66% | 11% | | | | | |
| 5 | 1 | AP | | 32% | 29% | | | | |
| 6 | 1 | AP | 98.10 | | | | | | |
| 7 | 1 | AP | 31% | | | | 8% | 17% | |
| 8 | 1 | AP | | | 19% | | | | |
| 9 | 1 | AP | 98% | | | | | | |
| 10 | 1 | AP | | 9% | 6% | | 16% | | |
| 11 | 2 | AP | 59% | | | 5% | | | 12% |
| 12 | 2 | AP | 53% | | | 23% | | | 14% |
| 13 | 2 | AP | 26% | 15% | | 14% | 5% | | 8% |
| 14 | 2 | AN | | 48% | | | | | |
| 15 | 2 | AN | | 6% | | | | | |
| 16 | 2 | AP | 38% | | | 12% | | | |
| 17 | 3 | AP | 6% | | 44% | | | | |
| 18 | 3 | AN | | 7% | | | | | |
| 19 | 3 | AP | | | | | | 13% | |
| 20 | 3 | AP | | | 44% | | | | |
| 21 | 3 | AN | | | | | | 17% | |
| 22 | 3 | AP | | 22% | 12% | | 18% | | |
| 23 | 3 | AP | 32% | | | 38% | | | |
| 24 | 3 | AP | | 5% | 25% | | | | |
| 25 | 3 | AP | | 10% | 10% | | | | |
| 26 | 3 | AP | | | 6% | | | | |
| 27 | 3 | AP | 54% | | 35% | | | | |
| 28 | 3 | AN | | | | | | 6% | |
| Sample ID | Group | Result | Escherichia | Enterococcus | Flavobacterium | Propionibacterium | Veillonella | Klebsiella | Sneathia |
| 1 | 1 | AN | | | | | | | |
| 2 | 1 | AN | | | | | | | |
| 3 | 1 | AN | | | | 91% | | | |
| 4 | 1 | AP | | | | | | | |
| 5 | 1 | AP | | | | | | | |
| 6 | 1 | AP | | | | | | | |
| 7 | 1 | AP | | | | | 6% | 15% | |
| 8 | 1 | AP | | | | | | | |
| 9 | 1 | AP | | | | | | | |
| 10 | 1 | AP | | | | | | | |
| 11 | 2 | AP | | | | | | | |
| 12 | 2 | AP | | | | | | | |
| 13 | 2 | AP | | | | | | | |
| 14 | 2 | AN | | | | | | | |
| 15 | 2 | AN | | | | | | | |
| 16 | 2 | AP | | | 6% | | | | |
| 17 | 3 | AP | | | | | | | |
| 18 | 3 | AN | | | | | | | |
| 19 | 3 | AP | | 10% | 27% | | | | |
| 20 | 3 | AP | 13% | | | | | | |
| 21 | 3 | AN | | | | | | | |
| 22 | 3 | AP | | | | | | | |
| 23 | 3 | AP | | | | | | | 19% |
| 24 | 3 | AP | | | | | | | |
| 25 | 3 | AP | | | | | | | |
| 26 | 3 | AP | | | | | | | |
| 27 | 3 | AP | | | | | | | |
| 28 | 3 | AN | | | | | | | |

TABLE 3
Summary of study samples

| | | | |
|---|---|---|---|
| Characteristics | Endometriosis | Control | p-value |

All samples (proliferative and secretory phases)

| | | | |
|---|---|---|---|
| Sample size | 166 | 100 | — |
| BMI | 21.48 (15.62-36.85) | 22.50 (17.1-31.22) | 0.83 |
| Age | 35.5 (20-51) | 38.5 (21-50) | 0.0002 |

Proliferative phase samples

| | | | |
|---|---|---|---|
| Sample size | 78 | 60 | — |
| BMI | 21.51 (16.21-36.85) | 22.50 (17.1-30.42) | 0.76 |
| Age | 36 (20-51) | 40.5 (24-50) | 0.026 |

Secretory phase samples

| | | | |
|---|---|---|---|
| Sample size | 88 | 40 | — |
| BMI | 21.45 (15.62-34.22) | 22.49 (18.22-31.22) | 0.94 |
| Age | 35 (21-51) | 37.5 (21-49) | 0.005 |

Median values are presented for each demographic parameter, with ranges shown in brackets. Statistical comparisons were conducted using T-tests.

TABLE 4
Selected taxa by random forest scoring

| | |
|---|---|
| Taxa | Importance_score |

Proliferative Cohort

| | |
|---|---|
| Staphylococcus sp.1 | 0.03939201 |
| Fenollaria sp.1 | 0.029557424 |
| Priestia sp.1 | 0.028568366 |
| Coprococcus sp.1 | 0.027826312 |
| Butyricimonas sp.1 | 0.026363355 |
| Corynebacterium sp.2 | 0.024171934 |
| Staphylococcus sp.2 | 0.021552353 |
| Finegoldia sp.1 | 0.020625778 |
| Mobiluncus sp.1 | 0.019181026 |
| Cutibacterium sp.1 | 0.018998935 |
| Peptoniphilus sp.1 | 0.01857177 |
| Priestia sp.2 | 0.018237289 |
| Veillonella sp.1 | 0.018100313 |
| Prevotella sp.2 | 0.017880653 |
| Anaerococcus sp.1 | 0.017697914 |
| Corynebacterium sp.1 | 0.017587248 |
| Prevotella sp.3 | 0.016008027 |
| Gardnerella sp.1 | 0.015508339 |

Secretory Cohort

| | |
|---|---|
| Gardnerella sp.1 | 0.044615353 |
| Lactobacillus sp.1 | 0.039103495 |
| Ureaplasma sp.2 | 0.028826797 |
| Lactobacillus sp.2 | 0.024935715 |
| Lawsonella sp.1 | 0.020598524 |
| Corynebacterium sp.3 | 0.018392541 |
| Priestia sp.1 | 0.01816104 |
| Lactobacillus sp.3 | 0.01711115 |
| Niallia sp.1 | 0.016852923 |
| Finegoldia sp.1 | 0.016236335 |
| Dialister sp.1 | 0.01592186 |
| Lactobacillus sp.4 | 0.015821996 |
| Ureaplasma sp.1 | 0.015624318 |

TABLE 5
Calculation of functional dysbiosis score (FDS) for each sample

| | | |
|---|---|---|
| Sample | Disease_status | FDS |

Proliferative phase

| | | |
|---|---|---|
| CEA25J0149 | 1 | −0.23868908 |
| CEA25J0150 | 1 | 3.003941491 |
| CEA25J0151 | 1 | −0.134063524 |
| SFBENDO307 | 1 | 2.951416502 |
| SFBENDO308 | 0 | −0.98290249 |
| SFBENDO313 | 0 | 1.698970004 |
| SFBENDO315 | 1 | 2.604894077 |
| SFBENDO319 | 1 | 2.598059314 |
| SFBENDO322 | 1 | 3.010196143 |
| SFBENDO323 | 0 | 0.717935436 |
| SFBENDO327 | 0 | 2.465079433 |
| SFBENDO328 | 1 | 2.640844764 |
| SFBENDO329 | 0 | 0.932851188 |
| SFBENDO334 | 1 | 2.245976485 |
| SFBENDO336 | 1 | −0.672248992 |
| SFBENDO337 | 1 | −0.402902425 |
| SFBENDO339 | 0 | 2.851693749 |
| SFBENDO340 | 1 | 0.019320225 |
| SFBENDO342 | 0 | 2.717892557 |
| SFBENDO343 | 0 | 1.009933471 |
| SFBENDO349 | 1 | 1.251422559 |
| SFBENDO353 | 1 | 2.301029996 |
| SFBENDO354 | 0 | 2.099458309 |
| SFBENDO358 | 1 | 2.484516838 |
| SFBENDO359 | 1 | 0.724887307 |
| SFBENDO361 | 0 | 2.939609546 |
| SFBENDO362 | 0 | 1.475940252 |
| SFBENDO363 | 0 | −0.006299472 |
| X233C241119T25 | 1 | 1.035440206 |
| X233C241119T27 | 1 | 0.146287806 |
| X233C241212T39 | 1 | 2.996531801 |
| X233C241212T40 | 0 | 1.116301872 |
| X233C241212T41 | 1 | 2.720345138 |
| X233C241212T42 | 1 | 1.365889295 |
| X233C241212T43 | 0 | 0.543608121 |
| X233C241212T44 | 0 | 2.538395525 |
| X233C241217T01 | 1 | 1.869329434 |
| X233C241217T02 | 0 | 2.461026377 |
| X233C241217T03 | 1 | 3.020504507 |
| X233C241217T04 | 1 | 3.012603367 |
| X233C241217T05 | 1 | 0.88063542 |
| X233C241217T06 | 1 | 2.997278597 |
| X233C241217T07 | 0 | 1.769583295 |
| X233C241217T08 | 1 | 2.508945326 |
| X233C241217T09 | 0 | 3.000587831 |
| X233C241217T10 | 0 | 2.975617913 |
| X233C241217T11 | 0 | 0.947542246 |
| X233C241217T12 | 0 | 1.724325584 |
| X233C241217T13 | 1 | 0.425291734 |
| X233C241217T14 | 1 | 2.552385657 |
| X233C241217T15 | 1 | 2.141314106 |
| X233C241217T16 | 1 | 2.337278569 |
| X233C241217T17 | 1 | 3.019846839 |
| X233C241217T18 | 1 | 0.710838428 |
| X233C241217T19 | 1 | 3.014962663 |
| X233C241217T20 | 0 | 0.05013802 |
| X233C241217T21 | 1 | 2.650917414 |
| X233C241217T22 | 1 | 1.760219386 |
| X233C241217T23 | 1 | 0.796196255 |
| X233C241217T24 | 0 | −0.780251832 |
| X233C241217T25 | 0 | 0.640267913 |
| X233C241217T26 | 0 | 2.11302716 |
| X233C241217T27 | 1 | 0.892961744 |
| X233C241217T28 | 0 | 1.223384793 |
| X233C241217T29 | 0 | 3.006815237 |
| X233C241217T30 | 0 | 2.452297671 |
| X233C241217T31 | 1 | 1.168038657 |
| X233C241217T32 | 0 | 2.776345117 |
| X233C241217T33 | 0 | 1.762948262 |
| X233C241217T34 | 1 | 2.965688329 |
| X233C241217T35 | 1 | 2.5064173 |
| X233C241217T36 | 0 | −0.350975662 |
| X233C241217T37 | 1 | 1.730662049 |
| X233C241217T38 | 0 | 0.198780877 |
| X233C241217T39 | 0 | 2.416713904 |
| X233C241217T40 | 0 | 2.146084876 |
| X233C241217T41 | 1 | 1.259128471 |
| X233C241217T42 | 0 | 1.366104301 |
| X233C241217T43 | 0 | 1.404417895 |
| X233C241217T44 | 0 | 2.988912391 |
| X233C241230T02 | 1 | 0.832169163 |
| X233C241230T03 | 0 | 0.61650078 |
| X233C241230T04 | 0 | 2.881715962 |
| X233C241230T05 | 0 | −0.2823976 |
| X233C241230T06 | 1 | 2.378641784 |
| X233C241230T07 | 1 | 1.598195837 |
| X233C241230T08 | 1 | 2.841734741 |
| X233C241230T09 | 1 | −0.931975845 |
| X233C241230T10 | 0 | 0.576230989 |
| X233C241230T11 | 1 | 2.146806064 |
| X233C241230T12 | 0 | −1.112582153 |
| X233C241230T13 | 0 | −0.41039375 |
| X233C241230T14 | 0 | 0.696068249 |
| X233C241230T15 | 1 | 2.979639412 |
| X233C241230T16 | 1 | 2.010464715 |
| X233C241230T17 | 0 | 0.817227954 |
| X233C241230T18 | 0 | 0.196786346 |
| X233C241230T19 | 1 | 2.845974084 |
| X233C241230T20 | 0 | 1.616502957 |
| X233C241230T21 | 0 | 1.59464773 |
| X233C241230T22 | 0 | 2.640624205 |
| X233C241230T23 | 0 | 3.019070198 |
| X233C241230T33 | 1 | 0.152967214 |
| X233C241230T38 | 1 | 0.705314041 |
| X233C241230T39 | 1 | 2.26491163 |
| X233C241230T40 | 1 | 2.979158264 |
| X233C241230T41 | 1 | 2.441403203 |
| X233C250106T02 | 0 | 0.879232935 |
| X233C250106T03 | 1 | 2.794360521 |
| X233C250106T04 | 0 | 2.83983604 |
| X233C250106T05 | 1 | 1.693558875 |
| X233C250106T06 | 1 | 2.269439489 |
| X233C250106T07 | 1 | 3.019730444 |
| X233C250106T08 | 1 | 3.010119075 |
| X233C250106T09 | 1 | 1.954007673 |
| X233C250106T10 | 1 | 2.252825621 |
| X233C250106T13 | 0 | 1.175740074 |
| X233C250106T16 | 1 | 2.078479442 |
| X233C250106T18 | 1 | 1.194687468 |
| X233C250106T20 | 1 | 2.173313183 |
| X233C250106T21 | 0 | 2.626018327 |
| X233C250106T22 | 0 | −1.400326964 |
| X233C250106T23 | 1 | −0.190184205 |
| X233C250106T24 | 0 | 1.536259218 |
| X233C250106T25 | 0 | 2.885640931 |
| X233C250106T26 | 1 | 2.778743196 |
| X233C250106T27 | 1 | 1.302941457 |
| X233C250106T28 | 1 | 2.617436486 |
| X233C250106T29 | 1 | 0.462011245 |
| X233C250106T30 | 1 | 2.546664416 |
| X233C250106T31 | 1 | 1.392238526 |
| X233C250106T32 | 1 | 1.828891141 |
| X233C250106T33 | 0 | 2.8801449 |
| X233C250106T35 | 1 | 2.697623108 |
| X233C250106T36 | 0 | 3.02117044 |
| X233C250106T38 | 1 | 1.341649896 |
| X233C250106T40 | 0 | 2.984314553 |
| X233C250106T42 | 1 | −0.703373885 |

Secretory phase

| | | |
|---|---|---|
| CEA25J0148 | 1 | 0.578934052 |
| SFBENDO306 | 0 | 2.242393039 |
| SFBENDO309 | 1 | 1.154270871 |
| SFBENDO316 | 1 | 2.232402319 |
| SFBENDO320 | 0 | 2.546493849 |
| SFBENDO321 | 1 | 2.70038833 |
| SFBENDO324 | 1 | 3.017656134 |
| SFBENDO326 | 0 | 1.61446494 |
| SFBENDO330 | 0 | 1.510358731 |
| SFBENDO331 | 0 | 0.079451358 |
| SFBENDO332 | 0 | 0.864398482 |
| SFBENDO333 | 1 | 1.039772977 |
| SFBENDO335 | 1 | −1.205647935 |
| SFBENDO338 | 1 | 2.776498862 |
| SFBENDO344 | 0 | 1.838804449 |
| SFBENDO346 | 1 | 2.184677022 |
| SFBENDO348 | 0 | 2.085424604 |
| SFBENDO350 | 1 | 1.208966189 |
| SFBENDO351 | 0 | 1.527847965 |
| SFBENDO355 | 0 | 2.90040443 |
| SFBENDO356 | 0 | −0.027167225 |
| SFBENDO357 | 0 | 1.877898254 |
| SFBENDO364 | 1 | −0.119124501 |
| SFBENDO365 | 1 | 2.185166399 |
| X233C241119T19 | 1 | 2.811345719 |
| X233C241205T02 | 1 | 1.271183377 |
| X233C241205T03 | 1 | 2.891718618 |
| X233C241205T05 | 1 | 1.885174101 |
| X233C241205T06 | 1 | 0.630461003 |
| X233C241205T07 | 1 | 2.562652882 |
| X233C241205T08 | 1 | 0.454944004 |
| X233C241205T09 | 1 | 1.378981912 |
| X233C241205T10 | 1 | 2.829901152 |
| X233C241205T11 | 1 | 2.434557697 |
| X233C241205T12 | 1 | −0.005539053 |
| X233C241205T13 | 1 | 0.962095313 |
| X233C241205T14 | 1 | 2.900776991 |
| X233C241205T15 | 0 | −0.16048259 |
| X233C241205T16 | 1 | 2.344172886 |
| X233C241205T17 | 1 | 2.542193611 |
| X233C241205T18 | 0 | 1.690034355 |
| X233C241205T19 | 0 | 3.012655313 |
| X233C241205T20 | 0 | 1.058323304 |
| X233C241205T21 | 1 | 0.114406655 |
| X233C241205T22 | 0 | 1.692321298 |
| X233C241205T23 | 1 | −1.250970773 |
| X233C241205T24 | 1 | 2.5026294 |
| X233C241205T25 | 1 | 1.246672333 |
| X233C241205T27 | 1 | 2.798808101 |
| X233C241205T28 | 0 | 1.123137015 |
| X233C241205T29 | 1 | 2.831501624 |
| X233C241205T30 | 1 | 0.618785115 |
| X233C241205T31 | 1 | 2.077563103 |
| X233C241205T32 | 1 | 1.515072527 |
| X233C241205T33 | 0 | 0.984861013 |
| X233C241205T34 | 0 | 2.421722118 |
| X233C241205T35 | 1 | 2.235509982 |
| X233C241205T36 | 1 | 2.595923758 |
| X233C241205T37 | 0 | 1.315504471 |
| X233C241205T38 | 1 | 2.75158412 |
| X233C241205T39 | 1 | 0.733695031 |
| X233C241205T40 | 1 | 2.55966021 |
| X233C241205T41 | 1 | 1.888976104 |
| X233C241205T42 | 1 | 0.756241083 |
| X233C241205T43 | 0 | 0.568850872 |
| X233C241205T44 | 1 | 2.463307505 |
| X233C241212T01 | 1 | 0.36565124 |
| X233C241212T03 | 0 | 2.954380136 |
| X233C241212T04 | 1 | 0.436801477 |
| X233C241212T05 | 1 | −2.90227492 |
| X233C241212T06 | 1 | 2.876435286 |
| X233C241212T07 | 1 | −1.326423647 |
| X233C241212T08 | 0 | 2.826718377 |
| X233C241212T09 | 1 | −0.244184775 |
| X233C241212T10 | 0 | 2.857532755 |
| X233C241212T11 | 0 | 2.4239612 |
| X233C241212T12 | 1 | 2.946736137 |
| X233C241212T13 | 1 | −0.346293237 |
| X233C241212T14 | 1 | 0.018622155 |
| X233C241212T15 | 1 | 1.121140656 |
| X233C241212T16 | 1 | 0.914653448 |
| X233C241212T17 | 1 | 1.835896871 |
| X233C241212T18 | 1 | 0.40252896 |
| X233C241212T19 | 1 | 2.902291697 |
| X233C241212T20 | 0 | 0.994284484 |
| X233C241212T21 | 0 | −0.885134966 |
| X233C241212T22 | 0 | 2.192370414 |
| X233C241212T23 | 1 | 2.789274691 |
| X233C241212T24 | 1 | −0.430991332 |
| X233C241212T25 | 1 | −2.117479631 |
| X233C241212T26 | 0 | −0.019142044 |
| X233C241212T27 | 0 | 2.952135972 |
| X233C241212T28 | 0 | 2.70821837 |
| X233C241212T29 | 1 | 0.983874781 |
| X233C241212T30 | 1 | 2.152348114 |
| X233C241212T31 | 1 | 2.689273684 |
| X233C241212T32 | 1 | 0.241567559 |
| X233C241212T33 | 1 | 2.37214085 |
| X233C241212T34 | 1 | 0.055594122 |
| X233C241212T35 | 1 | 2.473218051 |
| X233C241212T36 | 1 | 1.233842709 |
| X233C241212T37 | 1 | −1.315747846 |
| X233C241212T38 | 1 | 0.34634339 |
| X233C241230T24 | 1 | 2.395579309 |
| X233C241230T25 | 1 | −0.666810376 |
| X233C241230T26 | 1 | −0.334447998 |
| X233C241230T27 | 0 | 2.339243173 |
| X233C241230T28 | 1 | 0.866605755 |
| X233C241230T29 | 1 | 1.822378799 |
| X233C241230T30 | 1 | 2.981234117 |
| X233C241230T31 | 0 | 0.589041819 |
| X233C241230T32 | 0 | 2.911008409 |
| X233C241230T34 | 1 | 2.273747686 |
| X233C241230T35 | 1 | −0.783798099 |
| X233C241230T36 | 1 | 1.112232117 |
| X233C241230T37 | 1 | 0.600809638 |
| X233C250106T01 | 1 | 1.787004578 |
| X233C250106T11 | 0 | 2.997416479 |
| X233C250106T12 | 1 | 0.716569969 |
| X233C250106T14 | 0 | 0.41685455 |
| X233C250106T15 | 0 | 0.935281642 |
| X233C250106T17 | 0 | 2.960230857 |
| X233C250106T19 | 1 | 0.822790225 |
| X233C250106T34 | 1 | −1.050766311 |
| X233C250106T37 | 1 | 3.009524698 |
| X233C250106T39 | 1 | 2.524312645 |
| X233C250106T41 | 0 | −0.156724852 |
| X233C250106T44 | 1 | 2.556289058 |

TABLE 6
Feature set of proliferative cohort

| Fenollaria | Anaeroglobus | Anaerococcus | Coprococcus | Prevotella | Varibaculum | | |
|---|---|---|---|---|---|---|---|
| sp. 1 | sp. 1 | sp. 1 | sp. 1 | sp. 1 | sp. 1 | sample | Group |
| 0 | 0 | 0 | 0 | 0 | 0 | CEA25J0149 | 1 |
| 0 | 0 | 0 | 0 | 0 | 0 | CEA25J0150 | 1 |
| 0 | 0 | 0 | 0 | 0 | 0 | CEA25J0151 | 1 |
| 0 | 0 | 0 | 0 | 0 | 0 | SFBENDO307 | 1 |
| 0 | 0 | 0 | 0 | 0 | 0 | SFBENDO308 | 0 |
| 0 | 0 | 0 | 0 | 0 | 0 | SFBENDO313 | 0 |
| 0 | 0 | 0 | 0 | 0 | 0 | SFBENDO315 | 1 |
| 0 | 0 | 0 | 0.05868545 | 8.27464789 | 0 | SFBENDO319 | 1 |
| 0.13689036 | 0 | 0.01910098 | 0 | 0 | 0.1018719 | SFBENDO322 | 1 |
| 0 | 0 | 0 | 0 | 0 | 0 | SFBENDO323 | 0 |
| 0 | 0 | 0 | 0 | 0 | 0 | SFBENDO327 | 0 |
| 1.77124553 | 3.27868853 | 0 | 0 | 4.522329 | 1.43207085 | SFBENDO328 | 1 |
| 0 | 0 | 0 | 0 | 0 | 0 | SFBENDO329 | 0 |
| 0 | 0 | 0.05908669 | 0.01688191 | 0 | 0 | SFBENDO334 | 1 |
| 0 | 0 | 0 | 0 | 0 | 0 | SFBENDO336 | 1 |
| 0 | 0 | 0 | 0 | 0 | 0 | SFBENDO337 | 1 |
| 0.22480489 | 0 | 0 | 0.0042416 | 0 | 5.95096709 | SFBENDO339 | 0 |
| 0 | 0 | 0 | 0 | 0 | 0 | SFBENDO340 | 1 |
| 0 | 0 | 0 | 0.00440102 | 0 | 0.6073409 | SFBENDO342 | 0 |
| 0 | 0 | 0 | 0 | 0 | 0.01352997 | SFBENDO343 | 0 |
| 0 | 0 | 0 | 0.01120323 | 0 | 0 | SFBENDO349 | 1 |
| 2.59649123 | 0 | 5.96491228 | 0 | 0 | 0.28070175 | SFBENDO353 | 1 |
| 0 | 0 | 0 | 0 | 0 | 0 | SFBENDO354 | 0 |
| 1.14102336 | 0.59279729 | 0 | 0 | 3.15564272 | 1.97004814 | SFBENDO358 | 1 |
| 0 | 0 | 0 | 0 | 0 | 0 | SFBENDO359 | 1 |
| 0 | 0 | 0 | 0 | 0 | 0 | SFBENDO361 | 0 |
| 0 | 0 | 0 | 0 | 0 | 0 | SFBENDO362 | 0 |
| 0 | 0 | 0 | 0 | 0 | 0 | SFBENDO363 | 0 |
| 0 | 0 | 0 | 0 | 0 | 0 | X233C241119T25 | 1 |
| 0 | 0 | 0 | 0 | 0 | 0 | X233C241119T27 | 1 |
| 0 | 0 | 0 | 0 | 0 | 0 | X233C241212T39 | 1 |
| 0 | 0 | 0 | 0 | 0 | 0 | X233C241212T40 | 0 |
| 0 | 0 | 0 | 0 | 0 | 0 | X233C241212T41 | 1 |
| 0 | 0 | 0 | 0 | 0 | 0 | X233C241212T42 | 1 |
| 0 | 0 | 0 | 0 | 0 | 0 | X233C241212T43 | 0 |
| 0 | 0 | 0 | 0 | 0 | 0 | X233C241212T44 | 0 |
| 0 | 0 | 0 | 0.0160111 | 1.91599509 | 0 | X233C241217T01 | 1 |
| 0 | 0 | 0 | 0 | 0 | 0 | X233C241217T02 | 0 |
| 0 | 0 | 0 | 0.00231374 | 0 | 0 | X233C241217T03 | 1 |
| 0.13566789 | 0.11970696 | 0 | 0 | 0 | 0.33837167 | X233C241217T04 | 1 |
| 0 | 0 | 0 | 0 | 0 | 0 | X233C241217T05 | 1 |
| 0.06196663 | 0 | 0 | 0 | 0 | 0 | X233C241217T06 | 1 |
| 0 | 0 | 0 | 0 | 0 | 0 | X233C241217T07 | 0 |
| 0 | 0 | 0 | 0.00253004 | 0 | 0 | X233C241217T08 | 1 |
| 0 | 0 | 0 | 0 | 0 | 0 | X233C241217T09 | 0 |
| 0.1868918 | 0 | 0 | 0 | 2.0042534 | 0 | X233C241217T10 | 0 |
| 0 | 0 | 0 | 0 | 0 | 0 | X233C241217T11 | 0 |
| 0 | 0 | 0 | 0 | 0 | 0 | X233C241217T12 | 0 |
| 0 | 0 | 0 | 0 | 0 | 0 | X233C241217T13 | 1 |
| 0 | 0 | 0 | 0 | 4.82073643 | 0 | X233C241217T14 | 1 |
| 1.12014584 | 2.41778801 | 0.0071958 | 0 | 0.18948934 | 1.08176825 | X233C241217T15 | 1 |
| 0 | 0 | 0.04669624 | 0 | 0 | 1.25379407 | X233C241217T16 | 1 |
| 0 | 0 | 0 | 0 | 0 | 0 | X233C241217T17 | 1 |
| 0 | 0 | 0 | 0 | 0 | 0 | X233C241217T18 | 1 |
| 0.21271143 | 0 | 0 | 0 | 0 | 0.14479754 | X233C241217T19 | 1 |
| 0 | 0 | 0 | 0.0055701 | 0 | 0 | X233C241217T20 | 0 |
| 8.81648299 | 0 | 0 | 0.04791567 | 37.7575467 | 0 | X233C241217T21 | 1 |
| 0 | 0 | 0 | 0 | 0 | 0 | X233C241217T22 | 1 |
| 0.20528442 | 0 | 0 | 0.01986623 | 0.2119065 | 0.01324416 | X233C241217T23 | 1 |
| 0 | 0 | 0 | 0 | 0 | 0 | X233C241217T24 | 0 |
| 0 | 0 | 0 | 0 | 0 | 0 | X233C241217T25 | 0 |
| 0 | 0 | 0 | 0 | 0 | 0 | X233C241217T26 | 0 |
| 0 | 0 | 0 | 0.01040691 | 0 | 0 | X233C241217T27 | 1 |
| 0 | 0 | 0 | 0 | 0 | 0 | X233C241217T28 | 0 |
| 0 | 0 | 0 | 0 | 0 | 0 | X233C241217T29 | 0 |
| 0 | 0 | 0 | 0 | 0 | 0 | X233C241217T30 | 0 |
| 0 | 0 | 0 | 0.03320053 | 0 | 0 | X233C241217T31 | 1 |
| 0 | 0 | 0 | 0 | 0 | 0.67577494 | X233C241217T32 | 0 |
| 0 | 0 | 0 | 0 | 0 | 0 | X233C241217T33 | 0 |
| 0 | 0 | 0 | 0 | 0 | 0 | X233C241217T34 | 1 |
| 0 | 0 | 0.11723329 | 0 | 0 | 0 | X233C241217T35 | 1 |
| 0 | 0 | 0 | 0 | 0 | 0 | X233C241217T36 | 0 |
| 0 | 0 | 0 | 0 | 0 | 0 | X233C241217T37 | 1 |
| 0 | 0 | 0 | 0 | 0 | 0 | X233C241217T38 | 0 |
| 0 | 0 | 0 | 0 | 0 | 0 | X233C241217T39 | 0 |
| 0 | 0 | 0 | 0 | 0 | 0 | X233C241217T40 | 0 |
| 0 | 0 | 0 | 0 | 0 | 0 | X233C241217T41 | 1 |
| 0 | 0 | 0 | 0 | 0 | 0 | X233C241217T42 | 0 |
| 0 | 0 | 0 | 0 | 0 | 0 | X233C241217T43 | 0 |
| 0 | 0 | 0.02903725 | 0.00107545 | 0 | 0.24412802 | X233C241217T44 | 0 |
| 0 | 0 | 0 | 0 | 0 | 0 | X233C241230T02 | 1 |
| 0 | 0 | 0 | 0 | 0 | 0 | X233C241230T03 | 0 |
| 0 | 0 | 0 | 0 | 0 | 0.01350025 | X233C241230T04 | 0 |
| 0 | 0 | 0 | 0 | 0 | 0 | X233C241230T05 | 0 |
| 0 | 0.08315624 | 0 | 0 | 0 | 0 | X233C241230T06 | 1 |
| 0 | 0 | 0 | 0 | 0 | 0.1437833 | X233C241230T07 | 1 |
| 0 | 0 | 0 | 0 | 0 | 0 | X233C241230T08 | 1 |
| 0 | 0 | 0 | 0 | 0 | 0 | X233C241230T09 | 1 |
| 0 | 0 | 0 | 0 | 0 | 0 | X233C241230T10 | 0 |
| 0 | 0 | 0 | 0 | 0 | 0 | X233C241230T11 | 1 |
| 0 | 0 | 0 | 0 | 0 | 0 | X233C241230T12 | 0 |
| 0 | 0 | 0 | 0 | 0 | 0 | X233C241230T13 | 0 |
| 0 | 0 | 0 | 0 | 0 | 0 | X233C241230T14 | 0 |
| 0 | 0 | 0 | 0 | 0 | 0.16 | X233C241230T15 | 1 |
| 0 | 0 | 0.05558644 | 0 | 0 | 0 | X233C241230T16 | 1 |
| 0 | 0 | 0 | 0 | 0 | 0 | X233C241230T17 | 0 |
| 0 | 0 | 0 | 0 | 0 | 0 | X233C241230T18 | 0 |
| 5.54786151 | 0 | 0 | 0 | 4.10590631 | 0.47250509 | X233C241230T19 | 1 |
| 0 | 0 | 0 | 0 | 0 | 0 | X233C241230T20 | 0 |
| 0 | 0 | 0 | 0 | 0 | 0 | X233C241230T21 | 0 |
| 0 | 0 | 0.02308136 | 0 | 0 | 0 | X233C241230T22 | 0 |
| 0 | 0 | 0 | 0 | 0.06753335 | 0 | X233C241230T23 | 0 |
| 0 | 0 | 0 | 0 | 0 | 0 | X233C241230T33 | 1 |
| 2.05395463 | 0 | 0 | 0.01532802 | 0 | 0 | X233C241230T38 | 1 |
| 0 | 0 | 0.16079239 | 0 | 0 | 0 | X233C241230T39 | 1 |
| 0 | 0 | 0 | 0 | 0 | 0.83976396 | X233C241230T40 | 1 |
| 1.20845922 | 0.3021148 | 0 | 0 | 0 | 1.20845922 | X233C241230T41 | 1 |
| 0 | 0 | 0 | 0 | 0 | 0 | X233C250106T02 | 0 |
| 0 | 0 | 2.82510013 | 0 | 0 | 1.00133511 | X233C250106T03 | 1 |
| 0 | 0 | 0 | 0 | 0 | 0.36499069 | X233C250106T04 | 0 |
| 0 | 2.01700935 | 0 | 0 | 0 | 0.02932647 | X233C250106T05 | 1 |
| 0 | 0 | 2.15231788 | 0.08278146 | 0 | 0 | X233C250106T06 | 1 |
| 0 | 0 | 0 | 0 | 0 | 0 | X233C250106T07 | 1 |
| 0 | 0 | 0 | 0 | 0 | 0 | X233C250106T08 | 1 |
| 0 | 0 | 0 | 0 | 0 | 0 | X233C250106T09 | 1 |
| 1.08325596 | 0.30176416 | 0 | 0 | 3.24203033 | 0.06963788 | X233C250106T10 | 1 |
| 0 | 0 | 0 | 0 | 0 | 0 | X233C250106T13 | 0 |
| 0.11348857 | 0 | 0.15904968 | 0 | 0 | 0.72649254 | X233C250106T16 | 1 |
| 0 | 0 | 0 | 0 | 0.51596259 | 1.06417285 | X233C250106T18 | 1 |
| 0 | 0 | 0 | 0 | 0 | 0.40700041 | X233C250106T20 | 1 |
| 0 | 0 | 0 | 0 | 0.76142132 | 0 | X233C250106T21 | 0 |
| 0 | 0 | 0 | 0 | 0 | 0 | X233C250106T22 | 0 |
| 0 | 0 | 0 | 0 | 0 | 0 | X233C250106T23 | 1 |
| 0 | 0 | 0 | 0 | 0 | 0 | X233C250106T24 | 0 |
| 0 | 0 | 0 | 0 | 0.00727855 | 0 | X233C250106T25 | 0 |
| 0 | 0 | 0 | 0 | 0 | 0 | X233C250106T26 | 1 |
| 0 | 0 | 0 | 0 | 0 | 0 | X233C250106T27 | 1 |
| 0 | 0 | 0 | 0 | 0 | 0 | X233C250106T28 | 1 |
| 0 | 0 | 0 | 0 | 0 | 0 | X233C250106T29 | 1 |
| 0 | 0 | 0 | 0 | 0 | 0 | X233C250106T30 | 1 |
| 0 | 0 | 0.05182391 | 0 | 0 | 0 | X233C250106T31 | 1 |
| 0 | 0 | 0 | 0.06410256 | 0 | 0 | X233C250106T32 | 1 |
| 0 | 0 | 0 | 0 | 0 | 0 | X233C250106T33 | 0 |
| 0 | 0 | 0 | 0 | 4.53551913 | 0.91074681 | X233C250106T35 | 1 |
| 0 | 0 | 0 | 0 | 0 | 0 | X233C250106T36 | 0 |
| 0 | 0 | 0 | 0 | 0 | 0 | X233C250106T38 | 1 |
| 0 | 0 | 0 | 0 | 0 | 0 | X233C250106T40 | 0 |
| 0 | 0 | 0 | 0 | 0 | 0 | X233C250106T42 | 1 |
| Corynebacterium | Thalassobacillus | Staphylococcus | Priestia | Butyricimonas | Corynebacterium | | |
| sp. 1 | sp. 1 | sp. 1 | sp. 1 | sp. 1 | sp. 2 | Sample | Group |
| 0 | 0 | 0 | 0.00208368 | 0 | 0 | CEA25J0149 | 1 |
| 0 | 0 | 0 | 0.08176615 | 0 | 0 | CEA25J0150 | 1 |
| 0 | 0 | 0.09657948 | 0.00804829 | 0 | 0 | CEA25J0151 | 1 |
| 0 | 0 | 2.21138211 | 0.03252033 | 0 | 2.27642276 | SFBENDO307 | 1 |
| 0 | 0 | 0 | 0 | 0 | 0 | SFBENDO308 | 0 |
| 0 | 0 | 0.9478673 | 0.47393365 | 0 | 0 | SFBENDO313 | 0 |
| 0 | 0 | 0 | 0 | 0.01063842 | 0 | SFBENDO315 | 1 |
| 0 | 0 | 0 | 1.81924883 | 0 | 0 | SFBENDO319 | 1 |
| 0 | 0 | 0.01273399 | 0 | 0.0031835 | 0 | SFBENDO322 | 1 |
| 0 | 0 | 0 | 0 | 0 | 0.00235863 | SFBENDO323 | 0 |
| 0 | 0 | 0.00796305 | 0.00796305 | 0 | 0.39417105 | SFBENDO327 | 0 |
| 0 | 0.01884304 | 0.03768608 | 0.03768608 | 0 | 0.18843038 | SFBENDO328 | 1 |
| 0 | 0 | 0.07567159 | 0 | 0 | 0.06148316 | SFBENDO329 | 0 |
| 0 | 0 | 0 | 0 | 0 | 0 | SFBENDO334 | 1 |
| 0 | 0 | 0 | 0 | 0.03367155 | 0 | SFBENDO336 | 1 |
| 0 | 0 | 0 | 0 | 0.02100572 | 0 | SFBENDO337 | 1 |
| 0 | 0 | 0 | 0 | 0 | 0 | SFBENDO339 | 0 |
| 0 | 0 | 0.38308061 | 0.01596169 | 0 | 0 | SFBENDO340 | 1 |
| 0 | 0 | 0.15843676 | 0 | 0 | 0 | SFBENDO342 | 0 |
| 0 | 0 | 0.08117981 | 0 | 0 | 0 | SFBENDO343 | 0 |
| 0 | 0 | 0 | 0.01120323 | 0 | 0 | SFBENDO349 | 1 |
| 0 | 0 | 0.56140351 | 0.21052632 | 0 | 0 | SFBENDO353 | 1 |
| 0 | 0 | 1.41673932 | 0 | 0 | 0 | SFBENDO354 | 0 |
| 0 | 0 | 0 | 0 | 0 | 0 | SFBENDO358 | 1 |
| 0 | 0 | 0.11713031 | 0.02928258 | 0 | 0 | SFBENDO359 | 1 |
| 0 | 0 | 0.11670881 | 0.01945147 | 0.07780587 | 0 | SFBENDO361 | 0 |
| 0 | 0.01733403 | 1.69873462 | 0.03466805 | 0.01733403 | 0 | SFBENDO362 | 0 |
| 0 | 0 | 0 | 0 | 0 | 0 | SFBENDO363 | 0 |
| 0 | 0 | 0.07298382 | 0.01216397 | 0 | 1.39885659 | X233C241119T25 | 1 |
| 0 | 0 | 0.386349 | 0 | 0 | 0 | X233C241119T27 | 1 |
| 0 | 0 | 0 | 0.00419129 | 0 | 0 | X233C241212T39 | 1 |
| 0 | 0 | 0 | 0 | 0 | 0 | X233C241212T40 | 0 |
| 0 | 0 | 0 | 0 | 0 | 0 | X233C241212T41 | 1 |
| 0 | 0 | 0 | 0 | 0 | 0 | X233C241212T42 | 1 |
| 0 | 0 | 0.02380457 | 0 | 0 | 0 | X233C241212T43 | 0 |
| 0 | 0.04037142 | 0 | 0.04037142 | 0 | 0 | X233C241212T44 | 0 |
| 0 | 0 | 0 | 0 | 0.0160111 | 0 | X233C241217T01 | 1 |
| 0 | 0 | 0 | 0 | 0 | 0 | X233C241217T02 | 0 |
| 0 | 0 | 0 | 0 | 0 | 0 | X233C241217T03 | 1 |
| 0 | 0 | 0.00638437 | 0 | 0 | 0 | X233C241217T04 | 1 |
| 0 | 0 | 0 | 0 | 0 | 0 | X233C241217T05 | 1 |
| 0 | 0 | 0 | 0 | 0.0097842 | 0 | X233C241217T06 | 1 |
| 0 | 0 | 0 | 0.01141292 | 0.03423876 | 0 | X233C241217T07 | 0 |
| 0 | 0 | 0.0016867 | 0 | 0.00253004 | 0 | X233C241217T08 | 1 |
| 0 | 0 | 0 | 0 | 0 | 0 | X233C241217T09 | 0 |
| 0 | 0 | 0 | 0 | 0 | 0 | X233C241217T10 | 0 |
| 0 | 0 | 0.01702635 | 0 | 0.00425659 | 0 | X233C241217T11 | 0 |
| 0 | 0 | 0 | 35.1902923 | 0 | 0 | X233C241217T12 | 0 |
| 0 | 0 | 0 | 0.00830737 | 0 | 0 | X233C241217T13 | 1 |
| 0 | 0 | 0 | 0 | 0.0121124 | 0 | X233C241217T14 | 1 |
| 0 | 0 | 0.70998537 | 0.0023986 | 0 | 0 | X233C241217T15 | 1 |
| 0 | 0 | 6.23394817 | 0 | 0 | 4.88209199 | X233C241217T16 | 1 |
| 0 | 0 | 0 | 0 | 0.00342024 | 0 | X233C241217T17 | 1 |
| 0 | 0 | 0.00373507 | 0 | 0 | 0 | X233C241217T18 | 1 |
| 0 | 0 | 0.01537673 | 0 | 0 | 0.00640697 | X233C241217T19 | 1 |
| 0 | 0 | 0 | 0 | 0.0148536 | 0 | X233C241217T20 | 0 |
| 0 | 0 | 0 | 0.33540968 | 0.43124102 | 0 | X233C241217T21 | 1 |
| 0 | 0 | 0 | 0 | 0 | 0 | X233C241217T22 | 1 |
| 0 | 0 | 0 | 0 | 0.01986623 | 0 | X233C241217T23 | 1 |
| 0 | 0 | 0 | 0 | 0.0037696 | 0 | X233C241217T24 | 0 |
| 0 | 0 | 0 | 0 | 0 | 0 | X233C241217T25 | 0 |
| 0 | 0 | 0 | 0 | 0.00716236 | 0 | X233C241217T26 | 0 |
| 0 | 0 | 0 | 0 | 0.02081382 | 0 | X233C241217T27 | 1 |
| 0 | 0 | 0 | 0 | 0 | 0 | X233C241217T28 | 0 |
| 0 | 0 | 0 | 0 | 0 | 0 | X233C241217T29 | 0 |
| 0 | 0 | 0 | 0 | 0.11261261 | 0 | X233C241217T30 | 0 |
| 0 | 0 | 0.29880478 | 0.01660027 | 0.01660027 | 0 | X233C241217T31 | 1 |
| 0 | 0 | 0.20567063 | 0 | 0 | 0 | X233C241217T32 | 0 |
| 0 | 0 | 0 | 0 | 0.13623978 | 0 | X233C241217T33 | 0 |
| 0.00306736 | 0 | 0 | 0 | 0 | 0 | X233C241217T34 | 1 |
| 0 | 0 | 0.19839481 | 0 | 0.01352692 | 0 | X233C241217T35 | 1 |
| 0 | 0 | 0 | 0 | 0 | 0 | X233C241217T36 | 0 |
| 0 | 0 | 27.4021629 | 0 | 0 | 17.1555279 | X233C241217T37 | 1 |
| 0 | 0 | 0 | 0 | 0.00433792 | 0 | X233C241217T38 | 0 |
| 0 | 0 | 0 | 0 | 0 | 0 | X233C241217T39 | 0 |
| 0 | 0 | 0.0241955 | 0 | 0.01209775 | 0 | X233C241217T40 | 0 |
| 0 | 0 | 0 | 0.04258037 | 0 | 0 | X233C241217T41 | 1 |
| 0 | 0 | 0 | 0 | 0 | 0 | X233C241217T42 | 0 |
| 0 | 0 | 0 | 0 | 0.05272871 | 0 | X233C241217T43 | 0 |
| 0 | 0 | 0.00645272 | 0 | 0 | 0 | X233C241217T44 | 0 |
| 0 | 0 | 0 | 1.1816839 | 0 | 0 | X233C241230T02 | 1 |
| 0 | 0 | 0.72821847 | 0.0390117 | 0.0130039 | 0 | X233C241230T03 | 0 |
| 0 | 0 | 0.04418262 | 0 | 0 | 0.07854688 | X233C241230T04 | 0 |
| 0 | 0 | 0.02171308 | 0 | 0.00643351 | 0.03297172 | X233C241230T05 | 0 |
| 0 | 0 | 0.56361453 | 0 | 0 | 0 | X233C241230T06 | 1 |
| 0 | 0 | 11.1637461 | 0.00256756 | 0 | 0 | X233C241230T07 | 1 |
| 0 | 0 | 10.0091269 | 0.06084576 | 0 | 0 | X233C241230T08 | 1 |
| 0 | 0 | 0 | 0 | 0 | 0 | X233C241230T09 | 1 |
| 0 | 0 | 0.54904255 | 0 | 0.00675052 | 0.78756104 | X233C241230T10 | 0 |
| 0 | 0.00690036 | 0.76593983 | 0 | 0 | 0.10350538 | X233C241230T11 | 1 |
| 0 | 0 | 0 | 0 | 0 | 0 | X233C241230T12 | 0 |
| 0 | 0 | 0 | 0.04240283 | 0.01413428 | 0 | X233C241230T13 | 0 |
| 0 | 0.0554939 | 0 | 0.38845727 | 0 | 0 | X233C241230T14 | 0 |
| 0 | 0 | 0.24 | 0.16 | 0 | 2.48 | X233C241230T15 | 1 |
| 0.04168983 | 0 | 4.44691495 | 0.00694831 | 0.01389661 | 2.04974986 | X233C241230T16 | 1 |
| 0 | 0 | 0.00922777 | 0 | 0.00131825 | 0 | X233C241230T17 | 0 |
| 0 | 0 | 0 | 0.03115265 | 0 | 0 | X233C241230T18 | 0 |
| 0 | 0 | 1.24643585 | 0 | 0.02443992 | 0.51323829 | X233C241230T19 | 1 |
| 0 | 0 | 0.20494136 | 0 | 0 | 0 | X233C241230T20 | 0 |
| 0 | 0 | 0.13956734 | 9.07187718 | 0.13956734 | 0 | X233C241230T21 | 0 |
| 0 | 0 | 0.21927294 | 0 | 0.01154068 | 1.43104443 | X233C241230T22 | 0 |
| 0 | 0 | 0 | 0 | 0 | 0 | X233C241230T23 | 0 |
| 0 | 0 | 0 | 0 | 0 | 0 | X233C241230T33 | 1 |
| 0 | 0 | 0 | 0.01532802 | 0 | 0 | X233C241230T38 | 1 |
| 0 | 0 | 0.25941171 | 0 | 0 | 1.38495841 | X233C241230T39 | 1 |
| 0.02269632 | 0 | 0 | 0.02269632 | 0.02269632 | 4.67544258 | X233C241230T40 | 1 |
| 0 | 0 | 0 | 0 | 0.06042296 | 0 | X233C241230T41 | 1 |
| 0 | 0 | 0.03517461 | 0 | 0.00351746 | 0 | X233C250106T02 | 0 |
| 0 | 0 | 13.7142857 | 0.00534045 | 0 | 3.73564753 | X233C250106T03 | 1 |
| 0.00744879 | 0 | 0.02234637 | 0 | 0 | 0.18621974 | X233C250106T04 | 0 |
| 0 | 0 | 0 | 0 | 0 | 0 | X233C250106T05 | 1 |
| 0 | 0 | 0.33112583 | 0 | 0 | 0.99337748 | X233C250106T06 | 1 |
| 0 | 0 | 0.02901073 | 0 | 0 | 0 | X233C250106T07 | 1 |
| 0 | 0 | 0 | 0.0020247 | 0 | 0 | X233C250106T08 | 1 |
| 0 | 0 | 0.0748503 | 0.11227545 | 0 | 0 | X233C250106T09 | 1 |
| 0 | 0 | 0.01031672 | 0.00257918 | 0.01805427 | 0.07479625 | X233C250106T10 | 1 |
| 0 | 0 | 0.28601629 | 0 | 0 | 0 | X233C250106T13 | 0 |
| 0.15242261 | 0 | 2.08587026 | 0.00248515 | 0 | 2.83224401 | X233C250106T16 | 1 |
| 0 | 0 | 0 | 0 | 0 | 0.03224766 | X233C250106T18 | 1 |
| 0 | 0 | 1.3024013 | 0 | 0 | 0 | X233C250106T20 | 1 |
| 0 | 0 | 0.07809449 | 0 | 0.03904725 | 0.50761421 | X233C250106T21 | 0 |
| 0 | 0 | 0 | 0 | 0.00512487 | 0 | X233C250106T22 | 0 |
| 0 | 0 | 0.07763975 | 0 | 0 | 0 | X233C250106T23 | 1 |
| 0 | 0 | 0.13686652 | 0 | 0 | 0.02661293 | X233C250106T24 | 0 |
| 0 | 0 | 0.45127011 | 0 | 0.0145571 | 0.44399156 | X233C250106T25 | 0 |
| 0.47455767 | 0 | 0.53764916 | 0 | 0 | 0.11063869 | X233C250106T26 | 1 |
| 0 | 0 | 0.01037883 | 0 | 0 | 0 | X233C250106T27 | 1 |
| 0 | 0 | 0 | 0 | 0 | 0 | X233C250106T28 | 1 |
| 0 | 0 | 0 | 0 | 0 | 0 | X233C250106T29 | 1 |
| 0 | 0 | 0 | 36.9540556 | 0 | 0 | X233C250106T30 | 1 |
| 0 | 0 | 0 | 0 | 0 | 1.06095068 | X233C250106T31 | 1 |
| 0 | 0 | 0.38461539 | 0 | 0 | 0 | X233C250106T32 | 1 |
| 0 | 0 | 0 | 0.00072091 | 0 | 0 | X233C250106T33 | 0 |
| 0.16393443 | 0 | 1.20218579 | 0.89253188 | 0 | 7.70491803 | X233C250106T35 | 1 |
| 0 | 0 | 0 | 0 | 0.00455957 | 0 | X233C250106T36 | 0 |
| 0 | 0 | 0 | 10.3065539 | 0 | 0 | X233C250106T38 | 1 |
| 0 | 0.09950249 | 0.09950249 | 0.04975124 | 0 | 0 | X233C250106T40 | 0 |
| 0.02110684 | 0 | 0 | 0 | 0 | 0.00084427 | X233C250106T42 | 1 |
| Staphylococcus | Finegoldia | Mobiluncus | Cutibacterium | Peptoniphilus | Priestia | | |
| sp. 2 | sp. 1 | sp. 1 | sp. 1 | sp. 1 | sp. 2 | Sample | Group |
| 0 | 0 | 0 | 0 | 0 | 0 | CEA25J0149 | 1 |
| 0 | 0 | 0 | 0 | 0 | 0.12264922 | CEA25J0150 | 1 |
| 0 | 0 | 0 | 0 | 0 | 0.00402415 | CEA25J0151 | 1 |
| 0 | 0 | 0 | 0 | 0 | 0.03252033 | SFBENDO307 | 1 |
| 0 | 0 | 0 | 0 | 0 | 0.00187415 | SFBENDO308 | 0 |
| 0 | 0 | 0 | 0 | 0 | 2.36966825 | SFBENDO313 | 0 |
| 0 | 0 | 0 | 0 | 0 | 0 | SFBENDO315 | 1 |
| 0 | 0 | 0 | 0 | 0 | 0.05868545 | SFBENDO319 | 1 |
| 0 | 0.54437795 | 0.10823889 | 0 | 0.1973768 | 0 | SFBENDO322 | 1 |
| 0 | 0 | 0 | 0 | 0 | 0 | SFBENDO323 | 0 |
| 0 | 3.3126294 | 0 | 0 | 0.67287785 | 0 | SFBENDO327 | 0 |
| 0 | 4.46579989 | 2.07273413 | 0 | 0 | 0 | SFBENDO328 | 1 |
| 0 | 0 | 0.04256527 | 0 | 0 | 0 | SFBENDO329 | 0 |
| 0 | 0 | 0 | 0 | 0.27011058 | 0 | SFBENDO334 | 1 |
| 0 | 0 | 0 | 0 | 0 | 0 | SFBENDO336 | 1 |
| 0 | 0.00273988 | 0 | 0 | 0 | 0 | SFBENDO337 | 1 |
| 0 | 5.97217509 | 3.07516118 | 0 | 3.52901256 | 0 | SFBENDO339 | 0 |
| 0 | 0.01596169 | 0 | 0 | 0 | 0.01596169 | SFBENDO340 | 1 |
| 0.00880204 | 4.84552416 | 0.91101136 | 0 | 3.78927911 | 0.00440102 | SFBENDO342 | 0 |
| 0 | 0.0865918 | 0.03247193 | 0 | 0.03788391 | 0 | SFBENDO343 | 0 |
| 0 | 0 | 0 | 0 | 0 | 0 | SFBENDO349 | 1 |
| 0 | 0.70175439 | 0 | 0 | 5.2631579 | 0.14035088 | SFBENDO353 | 1 |
| 0.54489974 | 0 | 0 | 0.010898 | 0.010898 | 0 | SFBENDO354 | 0 |
| 0 | 0.95382421 | 0.24959886 | 0 | 0.57051168 | 0 | SFBENDO358 | 1 |
| 0 | 0 | 0 | 0 | 0 | 0 | SFBENDO359 | 1 |
| 0 | 0 | 0 | 0 | 0 | 0.01945147 | SFBENDO361 | 0 |
| 0.67602704 | 4.95753164 | 0 | 0 | 0.91870342 | 0.01733403 | SFBENDO362 | 0 |
| 0 | 0.00590179 | 0 | 0 | 0 | 0 | SFBENDO363 | 0 |
| 0 | 2.9923367 | 0 | 0 | 0 | 0 | X233C241119T25 | 1 |
| 0 | 0.03219575 | 0 | 0 | 0 | 0 | X233C241119T27 | 1 |
| 0 | 0.07125194 | 0 | 0 | 0 | 0.00419129 | X233C241212T39 | 1 |
| 0 | 0 | 0 | 0 | 0 | 0 | X233C241212T40 | 0 |
| 0 | 0 | 0 | 0 | 0 | 0 | X233C241212T41 | 1 |
| 0 | 0 | 0 | 0 | 0 | 0.00284455 | X233C241212T42 | 1 |
| 0 | 0 | 0 | 0 | 0 | 0 | X233C241212T43 | 0 |
| 0.02018571 | 0 | 0.14129996 | 0.30278563 | 0.06055713 | 0.02018571 | X233C241212T44 | 0 |
| 0 | 0 | 3.14884987 | 0 | 0.31488499 | 0 | X233C241217T01 | 1 |
| 0 | 5.2359882 | 0 | 0 | 24.0412979 | 0 | X233C241217T02 | 0 |
| 0 | 0 | 0.71726053 | 0 | 0.12956964 | 0 | X233C241217T03 | 1 |
| 0.00159609 | 0.35912087 | 0 | 0 | 0.64960976 | 0 | X233C241217T04 | 1 |
| 0 | 0 | 0 | 0 | 0 | 0 | X233C241217T05 | 1 |
| 0 | 0 | 5.13344567 | 0 | 0.49464587 | 0 | X233C241217T06 | 1 |
| 0 | 0 | 0 | 0 | 0 | 0 | X233C241217T07 | 0 |
| 0 | 0.00674679 | 0 | 0 | 0 | 0 | X233C241217T08 | 1 |
| 0 | 0 | 0 | 0 | 0 | 0 | X233C241217T09 | 0 |
| 0 | 0 | 0 | 0 | 0.19978089 | 0.02577818 | X233C241217T10 | 0 |
| 0.00425659 | 4.79291704 | 0 | 0 | 0 | 0 | X233C241217T11 | 0 |
| 0 | 0 | 0 | 0 | 0 | 0.60672918 | X233C241217T12 | 0 |
| 0 | 0 | 0 | 0 | 0.00415369 | 0.02907581 | X233C241217T13 | 1 |
| 0 | 4.13032946 | 0 | 0 | 3.80935078 | 0 | X233C241217T14 | 1 |
| 0 | 1.13933463 | 0.19908374 | 0 | 0.67400638 | 0 | X233C241217T15 | 1 |
| 0.06537474 | 7.0744805 | 0.38290918 | 0 | 1.8795237 | 0.00233481 | X233C241217T16 | 1 |
| 0 | 0.03591248 | 0 | 0 | 0.10688237 | 0 | X233C241217T17 | 1 |
| 0 | 0.01120521 | 0 | 0 | 0 | 0 | X233C241217T18 | 1 |
| 0 | 0.46899026 | 0.39338801 | 0 | 0.32034854 | 0 | X233C241217T19 | 1 |
| 0 | 0 | 0 | 0 | 0 | 0.0018567 | X233C241217T20 | 0 |
| 0 | 0 | 0 | 0 | 0 | 0.19166267 | X233C241217T21 | 1 |
| 0 | 2.23433012 | 0 | 0 | 0.15205271 | 0 | X233C241217T22 | 1 |
| 0 | 0 | 0.00662208 | 0 | 0 | 0.01986623 | X233C241217T23 | 1 |
| 0 | 0 | 0 | 0 | 0 | 0 | X233C241217T24 | 0 |
| 0 | 0.01073422 | 0 | 0 | 0 | 0 | X233C241217T25 | 0 |
| 0 | 0 | 0 | 0 | 0 | 0 | X233C241217T26 | 0 |
| 0 | 0.01561037 | 0.00520346 | 0 | 0.03122073 | 0 | X233C241217T27 | 1 |
| 0 | 0 | 0 | 0 | 0 | 0 | X233C241217T28 | 0 |
| 0 | 0 | 0 | 0 | 0 | 0 | X233C241217T29 | 0 |
| 0 | 0 | 0 | 0 | 0 | 0.02815315 | X233C241217T30 | 0 |
| 0 | 0 | 0 | 0 | 0 | 0 | X233C241217T31 | 1 |
| 0 | 7.1837814 | 0 | 0 | 0 | 0 | X233C241217T32 | 0 |
| 0 | 0 | 0 | 0 | 0.06811989 | 0 | X233C241217T33 | 0 |
| 0 | 0 | 0 | 0 | 0.04396548 | 0 | X233C241217T34 | 1 |
| 0 | 0.13526919 | 0 | 0 | 0.95139327 | 0 | X233C241217T35 | 1 |
| 0 | 0.04992805 | 0 | 0 | 0 | 0 | X233C241217T36 | 0 |
| 17.3578153 | 0.18672683 | 0 | 0 | 0.18672683 | 0 | X233C241217T37 | 1 |
| 0 | 0.03253443 | 0 | 0 | 0 | 0 | X233C241217T38 | 0 |
| 0 | 0.23273035 | 0 | 0 | 0 | 0 | X233C241217T39 | 0 |
| 0 | 0.86196468 | 0 | 0 | 0.10585531 | 0.00604888 | X233C241217T40 | 0 |
| 0 | 0 | 0 | 0 | 0 | 0.02129019 | X233C241217T41 | 1 |
| 0 | 0 | 0 | 0 | 2.19456584 | 0.00950029 | X233C241217T42 | 0 |
| 0.01318218 | 0 | 0 | 0 | 0 | 0 | X233C241217T43 | 0 |
| 0 | 3.26937968 | 0.00860363 | 0 | 2.41116751 | 0 | X233C241217T44 | 0 |
| 0.14771049 | 0 | 0 | 0 | 0 | 4.43131462 | X233C241230T02 | 1 |
| 0.62418726 | 0 | 0 | 0 | 0 | 0 | X233C241230T03 | 0 |
| 0.03313697 | 2.46563574 | 0 | 0 | 2.52700049 | 0 | X233C241230T04 | 0 |
| 0.01769214 | 0.12545336 | 0 | 0 | 0.00080419 | 0 | X233C241230T05 | 0 |
| 0.45273954 | 0.02771875 | 0 | 0 | 0.57285411 | 0.00923958 | X233C241230T06 | 1 |
| 7.52808268 | 0.08472944 | 0 | 0 | 0.07317543 | 0 | X233C241230T07 | 1 |
| 8.12290843 | 0 | 0 | 0 | 0 | 0.06084576 | X233C241230T08 | 1 |
| 0 | 0.00537731 | 0 | 0 | 0 | 0 | X233C241230T09 | 1 |
| 0.48153732 | 0 | 0 | 0 | 0 | 0.00450035 | X233C241230T10 | 0 |
| 0.55202871 | 3.92630417 | 0 | 0 | 1.40077284 | 0.01380072 | X233C241230T11 | 1 |
| 0.00350748 | 0 | 0 | 0 | 0 | 0 | X233C241230T12 | 0 |
| 0 | 0 | 0 | 0.01413428 | 0 | 0.04240283 | X233C241230T13 | 0 |
| 0 | 0 | 0 | 0.11098779 | 0 | 0.88790233 | X233C241230T14 | 0 |
| 0.24 | 0 | 0 | 0 | 0 | 0 | X233C241230T15 | 1 |
| 3.22401334 | 0.78515842 | 1.21595331 | 0 | 1.26459144 | 0 | X233C241230T16 | 1 |
| 0 | 0.01581903 | 0 | 0 | 0 | 0 | X233C241230T17 | 0 |
| 0 | 0 | 0 | 0.03115265 | 0 | 0 | X233C241230T18 | 0 |
| 1.04276986 | 10.7780041 | 0 | 0 | 5.89816701 | 0.00814664 | X233C241230T19 | 1 |
| 0.14801321 | 0.22771263 | 0 | 0 | 0.34156894 | 0.01138563 | X233C241230T20 | 0 |
| 0 | 0 | 0 | 0 | 3.14026518 | 0.13956734 | X233C241230T21 | 0 |
| 0.16156953 | 2.08886324 | 0 | 0.01154068 | 2.07732256 | 0 | X233C241230T22 | 0 |
| 0.00738646 | 0 | 0.04220834 | 0 | 0 | 0 | X233C241230T23 | 0 |
| 0 | 0 | 0 | 0 | 0.00181059 | 0 | X233C241230T33 | 1 |
| 0 | 0 | 0 | 0 | 0.01532802 | 0 | X233C241230T38 | 1 |
| 0.20795815 | 6.78758254 | 0 | 0 | 1.5328874 | 0 | X233C241230T39 | 1 |
| 0 | 0 | 0 | 0 | 0 | 0 | X233C241230T40 | 1 |
| 0 | 0.60422961 | 0 | 0 | 0 | 0.06042296 | X233C241230T41 | 1 |
| 0 | 0 | 0 | 0 | 0 | 0 | X233C250106T02 | 0 |
| 2.17623498 | 19.4392523 | 0.65687583 | 0 | 1.89319092 | 0 | X233C250106T03 | 1 |
| 0 | 16.0409683 | 0 | 0 | 5.59031657 | 0 | X233C250106T04 | 0 |
| 0 | 0.31933266 | 0 | 0 | 0.29652318 | 0 | X233C250106T05 | 1 |
| 0 | 2.81456954 | 0 | 0 | 6.12582782 | 0 | X233C250106T06 | 1 |
| 0 | 0.0435161 | 0 | 0.12329562 | 0.25384392 | 0 | X233C250106T07 | 1 |
| 0 | 0 | 0 | 0 | 0 | 0 | X233C250106T08 | 1 |
| 0 | 0 | 0 | 0 | 0 | 0 | X233C250106T09 | 1 |
| 0.00257918 | 0.84339214 | 0.51067781 | 0 | 0.70411637 | 0 | X233C250106T10 | 1 |
| 0 | 0 | 0 | 0 | 0 | 0 | X233C250106T13 | 0 |
| 0.0049703 | 0.82092829 | 0 | 0 | 3.02691419 | 0.00082838 | X233C250106T16 | 1 |
| 0 | 0.12899065 | 0 | 0 | 0.03224766 | 0 | X233C250106T18 | 1 |
| 0 | 0 | 0 | 0 | 0.2035002 | 0 | X233C250106T20 | 1 |
| 0 | 2.18664584 | 0 | 0 | 0.85903944 | 0 | X233C250106T21 | 0 |
| 0 | 0 | 0 | 0 | 0 | 0 | X233C250106T22 | 0 |
| 0 | 0.00970497 | 0 | 0 | 0 | 0 | X233C250106T23 | 1 |
| 0 | 0.43721249 | 0 | 0 | 0.01520739 | 0 | X233C250106T24 | 0 |
| 0.0145571 | 6.01936094 | 0.0436713 | 0 | 2.45287139 | 0 | X233C250106T25 | 0 |
| 0 | 0 | 0 | 0 | 0 | 0 | X233C250106T26 | 1 |
| 0 | 0 | 0 | 0 | 0 | 0 | X233C250106T27 | 1 |
| 0 | 0 | 0 | 0 | 0 | 0 | X233C250106T28 | 1 |
| 0 | 0 | 0 | 0 | 0 | 0 | X233C250106T29 | 1 |
| 0 | 0 | 0 | 0 | 0 | 0.76574022 | X233C250106T30 | 1 |
| 0 | 0.202977 | 1.75769441 | 0 | 0 | 0.00431866 | X233C250106T31 | 1 |
| 0 | 0 | 0 | 0 | 0 | 0 | X233C250106T32 | 1 |
| 0 | 0 | 0 | 0 | 0 | 0 | X233C250106T33 | 0 |
| 0.01821494 | 3.22404372 | 2.67759563 | 0 | 5.93806922 | 0.03642987 | X233C250106T35 | 1 |
| 0 | 0 | 0 | 0 | 0 | 0 | X233C250106T36 | 0 |
| 0 | 0 | 0 | 0 | 0 | 0.36997886 | X233C250106T38 | 1 |
| 0 | 0 | 0 | 0 | 0.14925373 | 0 | X233C250106T40 | 0 |
| 0 | 0.02701676 | 0 | 0 | 0 | 0 | X233C250106T42 | 1 |
| Veillonella | Prevotella | Prevotella | Gardnerella | | | | |
| sp. 1 | sp. 2 | sp. 3 | sp. 1 | | FDS | Sample | Group |
| 0 | 0 | 0 | 0 | | −0.2386891 | CEA25J0149 | 1 |
| 0 | 0 | 0 | 16.8438267 | | 3.00394149 | CEA25J0150 | 1 |
| 0 | 0 | 0 | 0 | | −0.1340635 | CEA25J0151 | 1 |
| 0 | 0 | 17.6585366 | 0 | | 2.9514165 | SFBENDO307 | 1 |
| 0 | 0 | 0 | 0 | | −0.9829025 | SFBENDO308 | 0 |
| 0 | 0 | 0 | 0 | | 1.69897 | SFBENDO313 | 0 |
| 30.814785 | 0 | 0.22813508 | 0 | | 2.60489408 | SFBENDO315 | 1 |
| 7.15962441 | 0 | 0.17605634 | 0.11737089 | | 2.59805931 | SFBENDO319 | 1 |
| 0.65898383 | 0.0986884 | 1.70317076 | 0 | | 3.01019614 | SFBENDO322 | 1 |
| 0 | 0 | 0.05188985 | 0 | | 0.71793544 | SFBENDO323 | 0 |
| 19.3303074 | 2.3212295 | 0.08759357 | 0.00398153 | | 2.46507943 | SFBENDO327 | 0 |
| 7.85754664 | 8.02713397 | 0 | 0.18843038 | | 2.64084476 | SFBENDO328 | 1 |
| 0.96008324 | 0 | 0 | 0.43511162 | | 0.93285119 | SFBENDO329 | 0 |
| 0 | 0 | 0.00844096 | 14.9742551 | | 2.24597649 | SFBENDO334 | 1 |
| 0 | 0 | 0.00336716 | 0.00336716 | | −0.672249 | SFBENDO336 | 1 |
| 0 | 0 | 0.0228323 | 0 | | −0.4029024 | SFBENDO337 | 1 |
| 4.03376315 | 47.7222599 | 0 | 0 | | 2.85169375 | SFBENDO339 | 0 |
| 0 | 0 | 0 | 0 | | 0.01932023 | SFBENDO340 | 1 |
| 0.62934601 | 9.47979931 | 11.8475486 | 0.42689904 | | 2.71789256 | SFBENDO342 | 0 |
| 0 | 0.10823975 | 0 | 0 | | 1.00993347 | SFBENDO343 | 0 |
| 0 | 0 | 0 | 1.59085817 | | 1.25142256 | SFBENDO349 | 1 |
| 0 | 0 | 0 | 0 | | 2.30103 | SFBENDO353 | 1 |
| 2.52833479 | 0 | 0 | 0 | | 2.09945831 | SFBENDO354 | 0 |
| 0 | 0.80228205 | 0 | 0 | | 2.48451684 | SFBENDO358 | 1 |
| 0 | 0 | 0 | 0 | | 0.72488731 | SFBENDO359 | 1 |
| 0 | 0 | 45.4580821 | 36.8410815 | | 2.93960955 | SFBENDO361 | 0 |
| 0 | 0 | 0 | 0 | | 1.47594025 | SFBENDO362 | 0 |
| 0 | 0 | 0 | 0 | | −0.0062995 | SFBENDO363 | 0 |
| 0 | 0 | 0 | 0 | | 1.03544021 | X233C241119T25 | 1 |
| 0 | 0 | 0 | 0 | | 0.14628781 | X233C241119T27 | 1 |
| 0 | 0.52181567 | 37.1390251 | 0 | | 2.9965318 | X233C241212T39 | 1 |
| 0 | 0.35565851 | 0 | 0.37609866 | | 1.11630187 | X233C241212T40 | 0 |
| 0 | 0 | 49.0909679 | 0 | | 2.72034514 | X233C241212T41 | 1 |
| 4.50576021 | 0 | 0 | 1.965581 | | 1.3658893 | X233C241212T42 | 1 |
| 0 | 0 | 0 | 0 | | 0.54360812 | X233C241212T43 | 0 |
| 0 | 0.36334275 | 1.00928543 | 25.2523214 | | 2.53839553 | X233C241212T44 | 0 |
| 0 | 0 | 0 | 1.65981747 | | 1.86932943 | X233C241217T01 | 1 |
| 0 | 0 | 0 | 0 | | 2.46102638 | X233C241217T02 | 0 |
| 0 | 0.2915317 | 0 | 19.4921333 | | 3.02050451 | X233C241217T03 | 1 |
| 0 | 3.95032959 | 7.78414441 | 60.6978118 | | 3.01260337 | X233C241217T04 | 1 |
| 0 | 0 | 0.68395161 | 0.00125496 | | 0.88063542 | X233C241217T05 | 1 |
| 0 | 0.54247975 | 0 | 23.6190683 | | 2.9972786 | X233C241217T06 | 1 |
| 0 | 0 | 0 | 0.02282584 | | 1.7695833 | X233C241217T07 | 0 |
| 0 | 0 | 13.2759857 | 0 | | 2.50894533 | X233C241217T08 | 1 |
| 0 | 0 | 0 | 95.0204163 | | 3.00058783 | X233C241217T09 | 0 |
| 1.93336341 | 0.23844815 | 0 | 64.5614487 | | 2.97561791 | X233C241217T10 | 0 |
| 0 | 0 | 0 | 0.42991529 | | 0.94754225 | X233C241217T11 | 0 |
| 0 | 0 | 0 | 0.22062879 | | 1.72432558 | X233C241217T12 | 0 |
| 0 | 0 | 0.02907581 | 0.10384216 | | 0.42529173 | X233C241217T13 | 1 |
| 1.62306202 | 8.49685078 | 5.31734496 | 1.1749031 | | 2.55238566 | X233C241217T14 | 1 |
| 2.09637572 | 0.13192296 | 8.02331438 | 0.0047972 | | 2.14131411 | X233C241217T15 | 1 |
| 0 | 2.3651646 | 4.68830259 | 0 | | 2.33727857 | X233C241217T16 | 1 |
| 0 | 0.97305709 | 12.9250712 | 0.02565177 | | 3.01984684 | X233C241217T17 | 1 |
| 0.00186754 | 0.00373507 | 0.14940286 | 0 | | 0.71083843 | X233C241217T18 | 1 |
| 0 | 3.69554075 | 5.28959508 | 0 | | 3.01496266 | X233C241217T19 | 1 |
| 0 | 0.0018567 | 0 | 0.0724113 | | 0.05013802 | X233C241217T20 | 0 |
| 0 | 0 | 0 | 0.19166267 | | 2.65091741 | X233C241217T21 | 1 |
| 0 | 0.37590809 | 2.77496199 | 0 | | 1.76021939 | X233C241217T22 | 1 |
| 0 | 0 | 0.03973247 | 0.09270909 | | 0.79619626 | X233C241217T23 | 1 |
| 0 | 0 | 0 | 0.00125653 | | −0.7802518 | X233C241217T24 | 0 |
| 0 | 0 | 0.0126859 | 0 | | 0.64026791 | X233C241217T25 | 0 |
| 0 | 0 | 0 | 0.00984825 | | 2.11302716 | X233C241217T26 | 0 |
| 0 | 0.02601728 | 0.09366219 | 0.29139349 | | 0.89296174 | X233C241217T27 | 1 |
| 0.10860856 | 0 | 0 | 1.52408083 | | 1.22338479 | X233C241217T28 | 0 |
| 0 | 0 | 0.01941289 | 88.1657354 | | 3.00681524 | X233C241217T29 | 0 |
| 0 | 0 | 0 | 7.77027027 | | 2.45229767 | X233C241217T30 | 0 |
| 0 | 0 | 0 | 0.34860558 | | 1.16803866 | X233C241217T31 | 1 |
| 12.4430733 | 38.2547378 | 0 | 0.02938152 | | 2.77634512 | X233C241217T32 | 0 |
| 0 | 0.06811989 | 0 | 0 | | 1.76294826 | X233C241217T33 | 0 |
| 0.15234551 | 0 | 13.278598 | 64.9339495 | | 2.96568833 | X233C241217T34 | 1 |
| 1.14527911 | 0 | 5.67228785 | 0.08116151 | | 2.5064173 | X233C241217T35 | 1 |
| 0 | 0 | 0 | 0.00881083 | | −0.3509757 | X233C241217T36 | 0 |
| 0 | 0.11670427 | 1.4782541 | 0.00778029 | | 1.73066205 | X233C241217T37 | 1 |
| 0.00867585 | 0.00361494 | 0.1091711 | 0 | | 0.19878088 | X233C241217T38 | 0 |
| 0 | 0 | 0 | 24.7434677 | | 2.4167139 | X233C241217T39 | 0 |
| 1.45172998 | 0 | 12.0705299 | 0.00604888 | | 2.14608488 | X233C241217T40 | 0 |
| 0 | 0 | 0 | 0.04258037 | | 1.25912847 | X233C241217T41 | 1 |
| 0 | 0 | 0 | 0.00950029 | | 1.3661043 | X233C241217T42 | 0 |
| 0 | 0 | 1.99050883 | 0.06591089 | | 1.4044179 | X233C241217T43 | 0 |
| 0.01075454 | 7.50559236 | 2.93921535 | 67.7234793 | | 2.98891239 | X233C241217T44 | 0 |
| 0 | 0 | 0 | 0.07385524 | | 0.83216916 | X233C241230T02 | 1 |
| 0 | 0 | 0 | 0 | | 0.61650078 | X233C241230T03 | 0 |
| 0.02086402 | 2.08271969 | 36.014973 | 0 | | 2.88171596 | X233C241230T04 | 0 |
| 0 | 0 | 0 | 0 | | −0.2823976 | X233C241230T05 | 0 |
| 0 | 2.28217685 | 7.73353044 | 7.4933013 | | 2.37864178 | X233C241230T06 | 1 |
| 0 | 0.83317286 | 0 | 0 | | 1.59819584 | X233C241230T07 | 1 |
| 0 | 0 | 0 | 53.6355339 | | 2.84173474 | X233C241230T08 | 1 |
| 0 | 0 | 0.0098584 | 0 | | −0.9319758 | X233C241230T09 | 1 |
| 0 | 0 | 0 | 0 | | 0.57623099 | X233C241230T10 | 0 |
| 0 | 0 | 7.63179685 | 0 | | 2.14680606 | X233C241230T11 | 1 |
| 0 | 0 | 0 | 0.00701496 | | −1.1125822 | X233C241230T12 | 0 |
| 0.6360424 | 0 | 0 | 0 | | −0.4103938 | X233C241230T13 | 0 |
| 0 | 0 | 0 | 0.0554939 | | 0.69606825 | X233C241230T14 | 0 |
| 0 | 0 | 0 | 80.08 | | 2.97963941 | X233C241230T15 | 1 |
| 0 | 1.34102279 | 4.33574208 | 0 | | 2.01046472 | X233C241230T16 | 1 |
| 1.00582668 | 0 | 0 | 0 | | 0.81722795 | X233C241230T17 | 0 |
| 0 | 0 | 0.03115265 | 0.03115265 | | 0.19678635 | X233C241230T18 | 0 |
| 0 | 7.31568228 | 2.89205703 | 1.14052953 | | 2.84597408 | X233C241230T19 | 1 |
| 0 | 0 | 2.39098258 | 0.56928157 | | 1.61650296 | X233C241230T20 | 0 |
| 0 | 0 | 0 | 0 | | 1.59464773 | X233C241230T21 | 0 |
| 0.02308136 | 0 | 15.4529717 | 0 | | 2.64062421 | X233C241230T22 | 0 |
| 0.01793855 | 0 | 0.22053858 | 99.0260426 | | 3.0190702 | X233C241230T23 | 0 |
| 0 | 0.00995827 | 0.07423435 | 0.02715891 | | 0.15296721 | X233C241230T33 | 1 |
| 0 | 0.27590435 | 0 | 0.04598406 | | 0.70531404 | X233C241230T38 | 1 |
| 0.57885259 | 0.63673784 | 3.08292599 | 0.65603293 | | 2.26491163 | X233C241230T39 | 1 |
| 0 | 0.40853382 | 0 | 85.4289605 | | 2.97915826 | X233C241230T40 | 1 |
| 0 | 4.7734139 | 4.83383686 | 6.34441088 | | 2.4414032 | X233C241230T41 | 1 |
| 0 | 0 | 0 | 0.69716071 | | 0.87923294 | X233C250106T02 | 0 |
| 1.38851802 | 12.552737 | 0 | 0.00267023 | | 2.79436052 | X233C250106T03 | 1 |
| 0 | 3.93668529 | 0.0037244 | 0.02607076 | | 2.83983604 | X233C250106T04 | 0 |
| 0 | 0.34540063 | 2.76320506 | 0 | | 1.69355888 | X233C250106T05 | 1 |
| 0 | 0 | 0 | 0.08278146 | | 2.26943949 | X233C250106T06 | 1 |
| 0 | 2.39338555 | 1.61734842 | 91.935016 | | 3.01973044 | X233C250106T07 | 1 |
| 0 | 0 | 9.09495849 | 64.5191334 | | 3.01011908 | X233C250106T08 | 1 |
| 0 | 0 | 0 | 8.23353293 | | 1.95400767 | X233C250106T09 | 1 |
| 0 | 0.38171877 | 6.49437739 | 1.58619622 | | 2.25282562 | X233C250106T10 | 1 |
| 0 | 0 | 1.05079898 | 0.23005658 | | 1.17574007 | X233C250106T13 | 0 |
| 0 | 3.77245955 | 0 | 0 | | 2.07847944 | X233C250106T16 | 1 |
| 0 | 0 | 0.74169623 | 0.03224766 | | 1.19468747 | X233C250106T18 | 1 |
| 0 | 1.22100122 | 0 | 0 | | 2.17331318 | X233C250106T20 | 1 |
| 0.01952362 | 0 | 1.54236626 | 8.43420539 | | 2.62601833 | X233C250106T21 | 0 |
| 0 | 0 | 0 | 0 | | −1.400327 | X233C250106T22 | 0 |
| 0 | 0 | 0 | 0 | | −0.1901842 | X233C250106T23 | 1 |
| 0 | 0.03421663 | 2.25449569 | 0.04562217 | | 1.53625922 | X233C250106T24 | 0 |
| 0 | 2.65667079 | 10.160856 | 8.13014048 | | 2.88564093 | X233C250106T25 | 0 |
| 0 | 0 | 0.90705436 | 11.5256252 | | 2.7787432 | X233C250106T26 | 1 |
| 0 | 0 | 0 | 1.9097042 | | 1.30294146 | X233C250106T27 | 1 |
| 0 | 0 | 0 | 0.73884262 | | 2.61743649 | X233C250106T28 | 1 |
| 0 | 0.0004546 | 0 | 0.24109166 | | 0.46201125 | X233C250106T29 | 1 |
| 0 | 0 | 0 | 31.4237096 | | 2.54666442 | X233C250106T30 | 1 |
| 0 | 0.16986727 | 0 | 0.0460657 | | 1.39223853 | X233C250106T31 | 1 |
| 0 | 0 | 0 | 0.32051282 | | 1.82889114 | X233C250106T32 | 1 |
| 0 | 0 | 0 | 55.2252988 | | 2.8801449 | X233C250106T33 | 0 |
| 3.22404372 | 12.6229508 | 10.3642987 | 0.01821494 | | 2.69762311 | X233C250106T35 | 1 |
| 0 | 0 | 21.963985 | 39.3446383 | | 3.02117044 | X233C250106T36 | 0 |
| 0 | 0 | 0 | 0 | | 1.3416499 | X233C250106T38 | 1 |
| 0 | 0 | 1.19402985 | 0 | | 2.98431455 | X233C250106T40 | 0 |
| 0 | 0 | 0 | 0 | | −0.7033739 | X233C250106T42 | 1 |

TABLE 7
Feature set of secretory cohort

| Ureaplasma | Niallia | Murdochiella | Gardnerella | Lactobacillus | | |
|---|---|---|---|---|---|---|
| sp. 1 | sp. 1 | sp. 1 | sp. 1 | sp. 1 | sample | group |
| 0 | 0 | 0 | 0 | 0 | CEA25J0148 | 1 |
| 0 | 0 | 0 | 0 | 22.2777992 | SFBENDO306 | 0 |
| 0 | 0 | 0 | 0 | 3.98976628 | SFBENDO309 | 1 |
| 0 | 0 | 0 | 0 | 47.4264383 | SFBENDO316 | 1 |
| 0 | 0 | 1.57188273 | 0.0176616 | 28.7707524 | SFBENDO320 | 0 |
| 0 | 0 | 0 | 0 | 0 | SFBENDO321 | 1 |
| 0.00803988 | 0 | 0 | 96.8028086 | 0.06967894 | SFBENDO324 | 1 |
| 0 | 0 | 0 | 0 | 0.08993884 | SFBENDO326 | 0 |
| 0 | 0 | 0 | 3.07320719 | 96.7146174 | SFBENDO330 | 0 |
| 0 | 0 | 0 | 0 | 0.05391954 | SFBENDO331 | 0 |
| 0 | 0 | 0.05922592 | 0 | 0.1451035 | SFBENDO332 | 0 |
| 0 | 0 | 0 | 0.05747126 | 27.1839081 | SFBENDO333 | 1 |
| 0 | 0 | 0 | 0 | 1.01240927 | SFBENDO335 | 1 |
| 0 | 0 | 0 | 0 | 0.06373486 | SFBENDO338 | 1 |
| 0 | 0 | 0 | 0 | 0.02836879 | SFBENDO344 | 0 |
| 0 | 0 | 0 | 0 | 65.8883249 | SFBENDO346 | 1 |
| 0 | 0 | 0 | 3.56885364 | 14.4196107 | SFBENDO348 | 0 |
| 0 | 0 | 0 | 0 | 71.8162839 | SFBENDO350 | 1 |
| 0 | 0 | 0 | 0 | 20.9982506 | SFBENDO351 | 0 |
| 0 | 0 | 0 | 12.1901872 | 0.02529085 | SFBENDO355 | 0 |
| 0 | 0 | 0 | 0 | 98.4114862 | SFBENDO356 | 0 |
| 0 | 0 | 0 | 0 | 0.06826871 | SFBENDO357 | 0 |
| 0 | 0 | 0 | 0 | 0 | SFBENDO364 | 1 |
| 0 | 0 | 0 | 0 | 0 | SFBENDO365 | 1 |
| 0 | 0 | 0 | 0 | 0 | X233C241119T19 | 1 |
| 0 | 0 | 0 | 0 | 69.8969646 | X233C241205T02 | 1 |
| 0 | 0 | 0 | 58.0891597 | 0 | X233C241205T03 | 1 |
| 0 | 0 | 0 | 0 | 72.7819549 | X233C241205T05 | 1 |
| 0 | 0 | 0 | 0 | 82.1749167 | X233C241205T06 | 1 |
| 0 | 0 | 0 | 0 | 60.3219856 | X233C241205T07 | 1 |
| 0 | 0 | 0 | 0.02065689 | 0.06197067 | X233C241205T08 | 1 |
| 0 | 0 | 0 | 0 | 31.4824121 | X233C241205T09 | 1 |
| 0 | 0 | 0 | 51.1699931 | 0 | X233C241205T10 | 1 |
| 0 | 0 | 0 | 0 | 3.9561842 | X233C241205T11 | 1 |
| 0 | 0 | 0 | 0 | 77.591119 | X233C241205T12 | 1 |
| 0 | 0 | 0 | 0.64510009 | 5.89128166 | X233C241205T13 | 1 |
| 0 | 0 | 0 | 68.1626055 | 0 | X233C241205T14 | 1 |
| 0 | 0 | 0 | 0 | 99.7987333 | X233C241205T15 | 0 |
| 0 | 0 | 0 | 0 | 72.1853498 | X233C241205T16 | 1 |
| 0 | 0 | 0 | 0 | 13.424366 | X233C241205T17 | 1 |
| 0 | 0 | 0 | 0.04242681 | 0 | X233C241205T18 | 0 |
| 0 | 0 | 0 | 88.4379786 | 0.02552323 | X233C241205T19 | 0 |
| 0 | 0 | 0 | 0 | 74.139435 | X233C241205T20 | 0 |
| 0 | 0 | 0 | 0 | 99.8678503 | X233C241205T21 | 1 |
| 0 | 0 | 0 | 0 | 2.65870863 | X233C241205T22 | 0 |
| 0 | 0 | 0 | 0 | 29.7296227 | X233C241205T23 | 1 |
| 0 | 0 | 0 | 1.48148148 | 0 | X233C241205T24 | 1 |
| 0 | 0 | 0 | 0 | 65.8952105 | X233C241205T25 | 1 |
| 0 | 0 | 0.34843206 | 32.8330206 | 0 | X233C241205T27 | 1 |
| 0 | 0 | 0 | 0.0464557 | 97.9012953 | X233C241205T28 | 0 |
| 0 | 0 | 0 | 0.02446184 | 0.1223092 | X233C241205T29 | 1 |
| 0 | 0 | 0 | 0 | 99.0403044 | X233C241205T30 | 1 |
| 0 | 0 | 0 | 3.01244863 | 0 | X233C241205T31 | 1 |
| 0 | 0 | 0 | 0 | 0 | X233C241205T32 | 1 |
| 0 | 0 | 0 | 0.82482054 | 0 | X233C241205T33 | 0 |
| 0 | 0 | 0 | 0 | 0 | X233C241205T34 | 0 |
| 0 | 0 | 0 | 0 | 59.3784278 | X233C241205T35 | 1 |
| 0 | 0 | 0 | 0 | 0 | X233C241205T36 | 1 |
| 0 | 0 | 0.02652392 | 0.18084491 | 33.8083526 | X233C241205T37 | 0 |
| 0 | 0 | 0 | 0.32693984 | 8.46992153 | X233C241205T38 | 1 |
| 0 | 0 | 0 | 0 | 95.8213097 | X233C241205T39 | 1 |
| 0 | 0 | 0 | 0 | 2.75869819 | X233C241205T40 | 1 |
| 0 | 0 | 0 | 0 | 0 | X233C241205T41 | 1 |
| 0 | 0 | 0 | 0 | 94.5835414 | X233C241205T42 | 1 |
| 0 | 0 | 0 | 0 | 3.23742459 | X233C241205T43 | 0 |
| 0 | 0 | 0 | 0 | 33.5883454 | X233C241205T44 | 1 |
| 0 | 0 | 0 | 0 | 10.2803738 | X233C241212T01 | 1 |
| 0 | 0 | 0 | 84.9946179 | 0.10764263 | X233C241212T03 | 0 |
| 0 | 0 | 0 | 0 | 99.722725 | X233C241212T04 | 1 |
| 0 | 0 | 0 | 0 | 99.9974953 | X233C241212T05 | 1 |
| 0 | 0 | 0 | 0 | 0 | X233C241212T06 | 1 |
| 0 | 0 | 0 | 0 | 99.8989423 | X233C241212T07 | 1 |
| 0 | 0 | 0 | 20.11271 | 0.01094272 | X233C241212T08 | 0 |
| 0 | 0 | 0 | 0 | 99.5391135 | X233C241212T09 | 1 |
| 0.00922116 | 0.02420554 | 0 | 0 | 0 | X233C241212T10 | 0 |
| 0 | 0.01786671 | 0 | 0 | 28.265142 | X233C241212T11 | 0 |
| 0 | 0 | 0 | 19.5805627 | 0 | X233C241212T12 | 1 |
| 0 | 0 | 0 | 0 | 99.0989752 | X233C241212T13 | 1 |
| 0 | 0 | 0 | 0.09230988 | 99.7585742 | X233C241212T14 | 1 |
| 0 | 0 | 0 | 0.00412057 | 1.29591858 | X233C241212T15 | 1 |
| 0 | 0 | 0 | 0.02211114 | 4.80522455 | X233C241212T16 | 1 |
| 0 | 0 | 0 | 0.73741369 | 6.55292619 | X233C241212T17 | 1 |
| 0 | 0 | 0 | 0.2406244 | 99.7593756 | X233C241212T18 | 1 |
| 0 | 0 | 0 | 0 | 16.9592782 | X233C241212T19 | 1 |
| 0 | 0 | 0 | 0.37547308 | 98.8399228 | X233C241212T20 | 0 |
| 0 | 0 | 0 | 0 | 0.99444155 | X233C241212T21 | 0 |
| 0 | 0 | 0 | 14.4824114 | 0 | X233C241212T22 | 0 |
| 0 | 0 | 0 | 8.48837683 | 0.00814233 | X233C241212T23 | 1 |
| 0 | 0 | 0 | 0 | 99.8575522 | X233C241212T24 | 1 |
| 0 | 0 | 0 | 0 | 39.1044646 | X233C241212T25 | 1 |
| 0 | 0 | 0 | 0 | 0 | X233C241212T26 | 0 |
| 0 | 0 | 0 | 82.271971 | 0.01210104 | X233C241212T27 | 0 |
| 0 | 0.00904159 | 0 | 0 | 1.85804702 | X233C241212T28 | 0 |
| 0 | 0 | 0 | 0.67341957 | 12.9240439 | X233C241212T29 | 1 |
| 0 | 0 | 0 | 0 | 0 | X233C241212T30 | 1 |
| 0 | 0 | 0 | 13.2178218 | 3.56435644 | X233C241212T31 | 1 |
| 0 | 0 | 0 | 0 | 96.7327481 | X233C241212T32 | 1 |
| 0 | 0 | 0 | 0 | 0 | X233C241212T33 | 1 |
| 0 | 0 | 0 | 0 | 99.5954788 | X233C241212T34 | 1 |
| 0 | 0 | 0 | 0 | 61.3925328 | X233C241212T35 | 1 |
| 0 | 0 | 0 | 0 | 11.3472391 | X233C241212T36 | 1 |
| 0 | 0 | 0 | 0 | 0 | X233C241212T37 | 1 |
| 0 | 0 | 0 | 0 | 0 | X233C241212T38 | 1 |
| 0 | 0 | 0 | 5.42096426 | 15.2314704 | X233C241230T24 | 1 |
| 0 | 0 | 0 | 0 | 88.3934581 | X233C241230T25 | 1 |
| 0 | 0 | 0 | 0 | 50.1980369 | X233C241230T26 | 1 |
| 0 | 0 | 0 | 13.4808013 | 0.06260434 | X233C241230T27 | 0 |
| 0 | 0 | 0 | 0.02172758 | 0.37540437 | X233C241230T28 | 1 |
| 0 | 0 | 0 | 0.57544757 | 17.7109974 | X233C241230T29 | 1 |
| 0 | 0 | 0 | 81.7143203 | 0.57426102 | X233C241230T30 | 1 |
| 0 | 0 | 0 | 0.24893267 | 88.0177733 | X233C241230T31 | 0 |
| 0 | 0 | 0.77864294 | 0 | 1.63144234 | X233C241230T32 | 0 |
| 0 | 0 | 0.03596152 | 0.0449519 | 0.01798076 | X233C241230T34 | 1 |
| 0 | 0 | 0 | 0 | 99.8454234 | X233C241230T35 | 1 |
| 0 | 0 | 0 | 0 | 87.2668763 | X233C241230T36 | 1 |
| 0.00274454 | 0 | 0 | 0 | 99.0490175 | X233C241230T37 | 1 |
| 0 | 0 | 0 | 5.09094409 | 80.6948321 | X233C250106T01 | 1 |
| 0 | 0 | 0 | 12.0182366 | 0 | X233C250106T11 | 0 |
| 0 | 0 | 0 | 0.24943746 | 7.71599016 | X233C250106T12 | 1 |
| 0.02329667 | 0 | 0 | 0.00038613 | 99.7039649 | X233C250106T14 | 0 |
| 0.01198682 | 0 | 0 | 0 | 0 | X233C250106T15 | 0 |
| 0 | 0 | 0 | 86.6316503 | 7.80243985 | X233C250106T17 | 0 |
| 0 | 0 | 0 | 0 | 87.0439173 | X233C250106T19 | 1 |
| 0 | 0 | 0 | 0 | 0.31400461 | X233C250106T34 | 1 |
| 0 | 0 | 0 | 0 | 0 | X233C250106T37 | 1 |
| 0 | 0 | 0 | 11.5090885 | 38.6941182 | X233C250106T39 | 1 |
| 0 | 0 | 0 | 0 | 0.89212385 | X233C250106T41 | 0 |
| 0 | 0 | 0 | 0 | 19.4364852 | X233C250106T44 | 1 |

| Lactobacillus | Lawsonella | Corynebacterium | Priestia | Lactobacillus | | |
|---|---|---|---|---|---|---|
| sp. 2 | sp. 1 | sp. 3 | sp. 1 | sp. 3 | Sample | Group |
| 0 | 0 | 0 | 0 | 99.179144 | CEA25J0148 | 1 |
| 54.7408344 | 0 | 0 | 0.00549662 | 0 | SFBENDO306 | 0 |
| 5.73226386 | 0 | 0 | 0 | 78.8964182 | SFBENDO309 | 1 |
| 0.04391744 | 0.22837066 | 0 | 0.00878349 | 2.41985068 | SFBENDO316 | 1 |
| 0.10596962 | 0.58283292 | 0 | 0.0176616 | 4.07983045 | SFBENDO320 | 0 |
| 0.00103289 | 0 | 0 | 0 | 51.3417204 | SFBENDO321 | 1 |
| 0.00803988 | 0 | 0 | 0 | 0 | SFBENDO324 | 1 |
| 0.00599592 | 0 | 0 | 0.01199185 | 94.5736899 | SFBENDO326 | 0 |
| 0.00266887 | 0 | 0.00133444 | 0 | 0 | SFBENDO330 | 0 |
| 0 | 0 | 0 | 0 | 97.5155537 | SFBENDO331 | 0 |
| 0 | 0 | 0 | 0 | 98.708875 | SFBENDO332 | 0 |
| 71.3362069 | 0.03113027 | 0 | 0.00239464 | 0.0598659 | SFBENDO333 | 1 |
| 18.8339967 | 0 | 0.00936549 | 0 | 80.1039569 | SFBENDO335 | 1 |
| 0.03186743 | 1.24282983 | 0.76481836 | 0.06373486 | 0.06373486 | SFBENDO338 | 1 |
| 2.80851064 | 0 | 0 | 0 | 90.2411348 | SFBENDO344 | 0 |
| 0.20304569 | 0 | 0 | 2.03045685 | 0 | SFBENDO346 | 1 |
| 0 | 13.0857967 | 0 | 0.14419611 | 0 | SFBENDO348 | 0 |
| 0 | 0 | 0 | 3.13152401 | 0 | SFBENDO350 | 1 |
| 73.7808878 | 0.01640061 | 0 | 0 | 0 | SFBENDO351 | 0 |
| 0.02529085 | 0.15174507 | 0.88517957 | 0 | 0 | SFBENDO355 | 0 |
| 0 | 0 | 0 | 0 | 0.01527417 | SFBENDO356 | 0 |
| 0.06826871 | 0 | 0 | 0.01365374 | 71.2179137 | SFBENDO357 | 0 |
| 0 | 0.01220078 | 0 | 0 | 99.8462702 | SFBENDO364 | 1 |
| 0 | 1.22171946 | 25.8823529 | 0 | 0 | SFBENDO365 | 1 |
| 0 | 0 | 0 | 0 | 37.6111194 | X233C241119T19 | 1 |
| 0 | 0.0278474 | 0 | 2.53411306 | 0 | X233C241205T02 | 1 |
| 0 | 0 | 0 | 2.59162557 | 0.00967025 | X233C241205T03 | 1 |
| 0 | 0 | 0.01879699 | 0 | 0 | X233C241205T05 | 1 |
| 0.15388561 | 0 | 0 | 0.0512952 | 10.3359836 | X233C241205T06 | 1 |
| 0 | 0 | 0 | 0.01677008 | 0 | X233C241205T07 | 1 |
| 0 | 0 | 0 | 0.02065689 | 95.0216897 | X233C241205T08 | 1 |
| 0.20100503 | 0.03768844 | 0 | 0.05025126 | 21.959799 | X233C241205T09 | 1 |
| 22.7116311 | 0 | 0 | 0.03441156 | 0.03441156 | X233C241205T10 | 1 |
| 0 | 0.80293523 | 0.06912687 | 0.01595236 | 0 | X233C241205T11 | 1 |
| 0 | 0 | 0 | 0.00982415 | 0.00982415 | X233C241205T12 | 1 |
| 33.3270088 | 0 | 0 | 0 | 58.3910445 | X233C241205T13 | 1 |
| 0 | 0 | 0 | 0 | 0 | X233C241205T14 | 1 |
| 0 | 0.0070373 | 0 | 0 | 0 | X233C241205T15 | 0 |
| 0.00608902 | 0 | 0 | 0 | 0 | X233C241205T16 | 1 |
| 7.39492818 | 1.36549034 | 0.37240646 | 2.961518 | 0.03546728 | X233C241205T17 | 1 |
| 4.49724226 | 0 | 0 | 11.1158252 | 0.08485363 | X233C241205T18 | 0 |
| 0 | 0 | 0.02552323 | 0.05104645 | 0 | X233C241205T19 | 0 |
| 0.01342012 | 0 | 0 | 0.00671006 | 21.0897135 | X233C241205T20 | 0 |
| 0 | 0 | 0 | 0 | 0 | X233C241205T21 | 1 |
| 2.44167119 | 0 | 2.65870863 | 0.16277808 | 41.9424851 | X233C241205T22 | 0 |
| 70.2624561 | 0 | 0 | 0 | 0.0013202 | X233C241205T23 | 1 |
| 0 | 0 | 0 | 5.18518519 | 0 | X233C241205T24 | 1 |
| 0 | 0 | 0 | 13.7576342 | 0.06428801 | X233C241205T25 | 1 |
| 0 | 0 | 0.15188064 | 0 | 12.2755294 | X233C241205T27 | 1 |
| 0 | 0 | 0 | 0 | 0 | X233C241205T28 | 0 |
| 0 | 0.34246575 | 0.02446184 | 0 | 0.02446184 | X233C241205T29 | 1 |
| 0.00279795 | 0.09792812 | 0 | 0 | 0.00139897 | X233C241205T30 | 1 |
| 0 | 0.17826928 | 0 | 0.00302151 | 35.3940053 | X233C241205T31 | 1 |
| 0.03459011 | 0 | 0 | 0 | 90.4704255 | X233C241205T32 | 1 |
| 0 | 0 | 0 | 0 | 98.8806829 | X233C241205T33 | 0 |
| 0 | 0 | 0 | 0.69860279 | 0 | X233C241205T34 | 0 |
| 0.10968921 | 0 | 0 | 3.25411335 | 0 | X233C241205T35 | 1 |
| 0 | 0 | 0 | 17.6716418 | 0.17910448 | X233C241205T36 | 1 |
| 0 | 0.10127315 | 0 | 0 | 61.5837191 | X233C241205T37 | 0 |
| 0 | 1.18570183 | 0.89363557 | 0 | 7.38884045 | X233C241205T38 | 1 |
| 0 | 0 | 0 | 0.02219756 | 0.00554939 | X233C241205T39 | 1 |
| 0 | 0 | 0 | 0 | 0.00140463 | X233C241205T40 | 1 |
| 0 | 0 | 0 | 2.34823882 | 85.9855109 | X233C241205T41 | 1 |
| 0 | 0 | 0 | 0 | 0 | X233C241205T42 | 1 |
| 46.580981 | 0.01513287 | 0 | 0 | 48.0095236 | X233C241205T43 | 0 |
| 0 | 0.47204362 | 0 | 0 | 13.5590462 | X233C241205T44 | 1 |
| 0 | 0 | 0 | 0.03115265 | 85.0778816 | X233C241212T01 | 1 |
| 0 | 0 | 0.31216362 | 0.01076426 | 0.07534984 | X233C241212T03 | 0 |
| 0 | 0 | 0 | 0 | 0.00723956 | X233C241212T04 | 1 |
| 0 | 0 | 0 | 0 | 0 | X233C241212T05 | 1 |
| 0 | 18.5683347 | 0.1253847 | 0 | 0.01139861 | X233C241212T06 | 1 |
| 0 | 0 | 0 | 0 | 0 | X233C241212T07 | 1 |
| 0 | 4.37708596 | 9.27942223 | 0 | 0 | X233C241212T08 | 0 |
| 0 | 0 | 0.09279594 | 0 | 0.0015466 | X233C241212T09 | 1 |
| 0 | 0.05878488 | 0 | 0 | 0 | X233C241212T10 | 0 |
| 0 | 0.41093443 | 0 | 0.01786671 | 0.01786671 | X233C241212T11 | 0 |
| 0 | 1.13043478 | 0 | 0 | 0 | X233C241212T12 | 1 |
| 0 | 0 | 0 | 0 | 0 | X233C241212T13 | 1 |
| 0 | 0 | 0.11006178 | 0 | 0 | X233C241212T14 | 1 |
| 0 | 0.09889363 | 0.06077838 | 0 | 96.2070173 | X233C241212T15 | 1 |
| 1.34246208 | 0 | 0 | 0 | 92.2966367 | X233C241212T16 | 1 |
| 3.59991956 | 2.54072535 | 0 | 0.00335188 | 76.1647784 | X233C241212T17 | 1 |
| 0 | 0 | 0 | 0 | 0 | X233C241212T18 | 1 |
| 0 | 0 | 2.69446477 | 0.01219215 | 0 | X233C241212T19 | 1 |
| 0 | 0 | 0.00747954 | 0 | 0.00074795 | X233C241212T20 | 0 |
| 0 | 0 | 0 | 0.00434254 | 98.9187077 | X233C241212T21 | 0 |
| 0 | 0.00434073 | 0 | 0 | 76.3100323 | X233C241212T22 | 0 |
| 0 | 2.32463461 | 0 | 0 | 31.2583968 | X233C241212T23 | 1 |
| 0 | 0 | 0 | 0 | 0 | X233C241212T24 | 1 |
| 33.1444018 | 0 | 0 | 0 | 27.7336938 | X233C241212T25 | 1 |
| 3.39059223 | 0 | 0 | 0 | 95.8603815 | X233C241212T26 | 0 |
| 0 | 0 | 0 | 0.00907578 | 0 | X233C241212T27 | 0 |
| 0 | 0.7278481 | 0 | 0 | 21.7359855 | X233C241212T28 | 0 |
| 2.09601841 | 0 | 0 | 0 | 82.9344258 | X233C241212T29 | 1 |
| 0 | 0 | 0 | 0.03619254 | 75.0995295 | X233C241212T30 | 1 |
| 1.28712871 | 0.04950495 | 6.23762376 | 0 | 14.8019802 | X233C241212T31 | 1 |
| 0 | 2.06964711 | 0 | 0 | 0.00581362 | X233C241212T32 | 1 |
| 1.6515816 | 0.73248111 | 0 | 0.01399645 | 68.1673976 | X233C241212T33 | 1 |
| 0 | 0 | 0 | 0 | 0.0022105 | X233C241212T34 | 1 |
| 0 | 0 | 2.13925328 | 0.0605449 | 0 | X233C241212T35 | 1 |
| 0 | 0 | 0 | 0.00357168 | 85.3703836 | X233C241212T36 | 1 |
| 0 | 0 | 0 | 0 | 99.9033321 | X233C241212T37 | 1 |
| 0 | 0 | 0 | 0 | 99.2642921 | X233C241212T38 | 1 |
| 0 | 2.59054929 | 0 | 0.04797314 | 0 | X233C241230T24 | 1 |
| 0 | 0 | 0 | 0 | 10.9267735 | X233C241230T25 | 1 |
| 49.439006 | 0 | 0 | 0 | 0.01986992 | X233C241230T26 | 1 |
| 0 | 0 | 0 | 0.08347245 | 0 | X233C241230T27 | 0 |
| 0.21003332 | 0 | 0.10018831 | 0 | 98.0022693 | X233C241230T28 | 1 |
| 0 | 0 | 0 | 0.3196931 | 3.13299233 | X233C241230T29 | 1 |
| 0 | 0.56519374 | 0 | 0 | 0 | X233C241230T30 | 1 |
| 11.5740307 | 0 | 0 | 0 | 0.00133835 | X233C241230T31 | 0 |
| 1.94042764 | 0 | 0 | 0.02471882 | 0.29662588 | X233C241230T32 | 0 |
| 0 | 0.35961521 | 0 | 0 | 71.5454464 | X233C241230T34 | 1 |
| 0 | 0 | 0 | 0 | 0.00220824 | X233C241230T35 | 1 |
| 0 | 0.1828421 | 0 | 0.02194105 | 0 | X233C241230T36 | 1 |
| 0 | 0 | 0 | 0 | 0 | X233C241230T37 | 1 |
| 0.00962371 | 0 | 0 | 0 | 11.0191512 | X233C250106T01 | 1 |
| 0 | 0 | 0 | 0 | 0 | X233C250106T11 | 0 |
| 0.00087216 | 0 | 0 | 0 | 91.4441208 | X233C250106T12 | 1 |
| 0 | 0 | 0 | 0 | 0.03771229 | X233C250106T14 | 0 |
| 0 | 0 | 0 | 5.83158526 | 87.6835481 | X233C250106T15 | 0 |
| 0 | 0 | 0 | 0.03388682 | 0.00847171 | X233C250106T17 | 0 |
| 0 | 0 | 0 | 0 | 0.02857959 | X233C250106T19 | 1 |
| 0 | 0 | 0 | 0 | 99.1207871 | X233C250106T34 | 1 |
| 0.05138746 | 0.30832477 | 0 | 0.05138746 | 0.10277492 | X233C250106T37 | 1 |
| 0 | 0.06252512 | 0 | 0 | 21.6158278 | X233C250106T39 | 1 |
| 3.93602168 | 0 | 0 | 0 | 94.3474869 | X233C250106T41 | 0 |
| 0 | 0.0159185 | 0 | 0 | 36.1668259 | X233C250106T44 | 1 |

| Finegoldia | Dialister | Lactobacillus | Ureaplasma | | | |
|---|---|---|---|---|---|---|
| sp. 1 | sp. 1 | sp. 4 | sp. 2 | FDS | Sample | Group |
| 0 | 0 | 0 | 0 | 0.57893405 | CEA25J0148 | 1 |
| 3.79266751 | 0 | 0 | 0 | 2.24239304 | SFBENDO306 | 0 |
| 0.13137879 | 0 | 0 | 0 | 1.15427087 | SFBENDO309 | 1 |
| 5.23056654 | 0.06587615 | 0.09661836 | 0.02635046 | 2.23240232 | SFBENDO316 | 1 |
| 7.55916637 | 0.03532321 | 0 | 0 | 2.54649385 | SFBENDO320 | 0 |
| 0.32535945 | 0 | 0 | 0 | 2.70038833 | SFBENDO321 | 1 |
| 0.42611352 | 0 | 0 | 0.35107466 | 3.01765613 | SFBENDO324 | 1 |
| 0.93536395 | 0 | 0 | 0 | 1.61446494 | SFBENDO326 | 0 |
| 0 | 0 | 0 | 0 | 1.51035873 | SFBENDO330 | 0 |
| 0 | 0 | 1.02447117 | 0 | 0.07945136 | SFBENDO331 | 0 |
| 0.12881637 | 0 | 0.16139063 | 0 | 0.86439848 | SFBENDO332 | 0 |
| 0 | 0 | 0 | 0 | 1.03977298 | SFBENDO333 | 1 |
| 0.01592133 | 0 | 0 | 0 | −1.2056479 | SFBENDO335 | 1 |
| 12.0458891 | 0.63734863 | 0 | 0 | 2.77649886 | SFBENDO338 | 1 |
| 0 | 0 | 0 | 0 | 1.83880445 | SFBENDO344 | 0 |
| 9.94923858 | 0 | 0 | 0 | 2.18467702 | SFBENDO346 | 1 |
| 0 | 0 | 0 | 0 | 2.0854246 | SFBENDO348 | 0 |
| 0 | 0 | 0 | 0 | 1.20896619 | SFBENDO350 | 1 |
| 1.57992565 | 0.03280123 | 0 | 0 | 1.52784797 | SFBENDO351 | 0 |
| 11.9878604 | 1.34041477 | 0 | 0 | 2.90040443 | SFBENDO355 | 0 |
| 0 | 0 | 0 | 0 | −0.0271672 | SFBENDO356 | 0 |
| 0.27307482 | 0 | 0 | 0 | 1.87789825 | SFBENDO357 | 0 |
| 0 | 0 | 0 | 0.05124326 | −0.1191245 | SFBENDO364 | 1 |
| 1.53846154 | 0 | 0 | 0 | 2.1851664 | SFBENDO365 | 1 |
| 0.45195487 | 0.04652477 | 0 | 0 | 2.81134572 | X233C241119T19 | 1 |
| 0 | 0 | 0 | 0 | 1.27118338 | X233C241205T02 | 1 |
| 0 | 0 | 0 | 0 | 2.89171862 | X233C241205T03 | 1 |
| 6.16541353 | 0 | 0 | 0 | 1.8851741 | X233C241205T05 | 1 |
| 0 | 0 | 1.3593229 | 0.12823801 | 0.630461 | X233C241205T06 | 1 |
| 0.13416066 | 0 | 0 | 0 | 2.56265288 | X233C241205T07 | 1 |
| 0.43379467 | 0 | 0.45445156 | 0 | 0.454944 | X233C241205T08 | 1 |
| 0 | 0 | 0 | 0.05025126 | 1.37898191 | X233C241205T09 | 1 |
| 0 | 0 | 0 | 0 | 2.82990115 | X233C241205T10 | 1 |
| 5.8119749 | 0.03190471 | 0 | 0 | 2.4345577 | X233C241205T11 | 1 |
| 0 | 0 | 0 | 0 | −0.0055391 | X233C241205T12 | 1 |
| 0 | 0 | 0 | 0 | 0.96209531 | X233C241205T13 | 1 |
| 0.16565766 | 0.08634278 | 0 | 0 | 2.90077699 | X233C241205T14 | 1 |
| 0.03940887 | 0 | 0 | 0 | −0.1604826 | X233C241205T15 | 0 |
| 0 | 1.60750167 | 0 | 0 | 2.34417289 | X233C241205T16 | 1 |
| 16.1553467 | 0.24827097 | 0 | 0.12413549 | 2.54219361 | X233C241205T17 | 1 |
| 0 | 0 | 0 | 0 | 1.69003436 | X233C241205T18 | 0 |
| 0.81674324 | 1.63348647 | 0 | 0 | 3.01265531 | X233C241205T19 | 0 |
| 0.01342012 | 0 | 0 | 0 | 1.0583233 | X233C241205T20 | 0 |
| 0.00718205 | 0 | 0 | 0 | 0.11440666 | X233C241205T21 | 1 |
| 0 | 0 | 5.48019533 | 0 | 1.6923213 | X233C241205T22 | 0 |
| 0 | 0 | 0 | 0 | −1.2509708 | X233C241205T23 | 1 |
| 4.22222222 | 0.07407407 | 0 | 0 | 2.5026294 | X233C241205T24 | 1 |
| 0 | 0 | 0.03214401 | 0 | 1.24667233 | X233C241205T25 | 1 |
| 8.56785491 | 0.01786831 | 0 | 0 | 2.7988081 | X233C241205T27 | 1 |
| 0.20495163 | 0 | 0 | 0.05328742 | 1.12313702 | X233C241205T28 | 0 |
| 0.2446184 | 1.22309198 | 0 | 0 | 2.83150162 | X233C241205T29 | 1 |
| 0 | 0.01398973 | 0 | 0 | 0.61878512 | X233C241205T30 | 1 |
| 3.43243897 | 0.16618323 | 1.49564902 | 0 | 2.0775631 | X233C241205T31 | 1 |
| 0 | 0 | 0.05188516 | 0 | 1.51507253 | X233C241205T32 | 1 |
| 0 | 0 | 0.07132339 | 0.08857905 | 0.98486101 | X233C241205T33 | 0 |
| 0 | 0 | 0 | 0 | 2.42172212 | X233C241205T34 | 0 |
| 5.44789762 | 0 | 0 | 0 | 2.23550998 | X233C241205T35 | 1 |
| 0 | 0 | 0 | 0 | 2.59592376 | X233C241205T36 | 1 |
| 0.70650077 | 0.07716049 | 0 | 0.11815201 | 1.31550447 | X233C241205T37 | 0 |
| 14.0671317 | 0.98517873 | 0.07410636 | 0.03487358 | 2.75158412 | X233C241205T38 | 1 |
| 0 | 0 | 0 | 0 | 0.73369503 | X233C241205T39 | 1 |
| 0.02106949 | 0.3244701 | 0 | 0 | 2.55966021 | X233C241205T40 | 1 |
| 0.04996253 | 0 | 0 | 0 | 1.8889761 | X233C241205T41 | 1 |
| 0 | 0 | 0 | 0 | 0.75624108 | X233C241205T42 | 1 |
| 0.20681584 | 0 | 1.13698271 | 0 | 0.56885087 | X233C241205T43 | 0 |
| 6.4783918 | 0.34996338 | 0 | 0 | 2.46330751 | X233C241205T44 | 1 |
| 0 | 0 | 0 | 0 | 0.36565124 | X233C241212T01 | 1 |
| 0 | 0 | 0 | 0 | 2.95438014 | X233C241212T03 | 0 |
| 0.00579165 | 0.00579165 | 0 | 0.0702237 | 0.43680148 | X233C241212T04 | 1 |
| 0 | 0 | 0 | 0 | −2.9022749 | X233C241212T05 | 1 |
| 6.77077397 | 0.21657358 | 0 | 0.22797219 | 2.87643529 | X233C241212T06 | 1 |
| 0 | 0 | 0 | 0 | −1.3264236 | X233C241212T07 | 1 |
| 8.87454177 | 0.07112765 | 0 | 0 | 2.82671838 | X233C241212T08 | 0 |
| 0.17012589 | 0 | 0 | 0 | −0.2441848 | X233C241212T09 | 1 |
| 0.63280196 | 0 | 0 | 0.10143274 | 2.85753276 | X233C241212T10 | 0 |
| 0.19653386 | 0 | 0 | 0 | 2.4239612 | X233C241212T11 | 0 |
| 0.05626599 | 2.58823529 | 0 | 0 | 2.94673614 | X233C241212T12 | 1 |
| 0.74626866 | 0 | 0 | 0 | −0.3462932 | X233C241212T13 | 1 |
| 0.01420152 | 0 | 0 | 0 | 0.01862216 | X233C241212T14 | 1 |
| 0.19057626 | 0.1349486 | 0.28637946 | 0.02163298 | 1.12114066 | X233C241212T15 | 1 |
| 0.11292475 | 0 | 0 | 0 | 0.91465345 | X233C241212T16 | 1 |
| 0 | 0.8212107 | 1.10276865 | 0 | 1.83589687 | X233C241212T17 | 1 |
| 0 | 0 | 0 | 0 | 0.40252896 | X233C241212T18 | 1 |
| 0.17069008 | 0 | 0 | 0 | 2.9022917 | X233C241212T19 | 1 |
| 0.09798202 | 0 | 0 | 0 | 0.99428448 | X233C241212T20 | 0 |
| 0 | 0 | 0 | 0 | −0.885135 | X233C241212T21 | 0 |
| 0.04253915 | 0.02604438 | 5.69937841 | 0 | 2.19237041 | X233C241212T22 | 0 |
| 1.64475023 | 0.3053373 | 0 | 0 | 2.78927469 | X233C241212T23 | 1 |
| 0 | 0 | 0 | 0.00890299 | −0.4309913 | X233C241212T24 | 1 |
| 0 | 0 | 0 | 0 | −2.1174796 | X233C241212T25 | 1 |
| 0.01373215 | 0 | 0 | 0 | −0.019142 | X233C241212T26 | 0 |
| 0 | 0 | 0 | 0 | 2.95213597 | X233C241212T27 | 0 |
| 15.244123 | 3.58951175 | 0 | 0.02260398 | 2.70821837 | X233C241212T28 | 0 |
| 0.03928281 | 0 | 0 | 0 | 0.98387478 | X233C241212T29 | 1 |
| 1.99058994 | 0 | 0 | 0 | 2.15234811 | X233C241212T30 | 1 |
| 0 | 0.0990099 | 0.0990099 | 0 | 2.68927368 | X233C241212T31 | 1 |
| 0.94761932 | 0.00581362 | 0 | 0 | 0.24156756 | X233C241212T32 | 1 |
| 0.4292246 | 0 | 0 | 0 | 2.37214085 | X233C241212T33 | 1 |
| 0.21441834 | 0 | 0 | 0.05673613 | 0.05559412 | X233C241212T34 | 1 |
| 0 | 0 | 0.18163471 | 0 | 2.47321805 | X233C241212T35 | 1 |
| 0.01071505 | 0 | 0.16072577 | 0 | 1.23384271 | X233C241212T36 | 1 |
| 0 | 0 | 0 | 0 | −1.3157478 | X233C241212T37 | 1 |
| 0.09020491 | 0.00993082 | 0 | 0 | 0.34634339 | X233C241212T38 | 1 |
| 4.10170305 | 0 | 0 | 0 | 2.39557931 | X233C241230T24 | 1 |
| 0.02187374 | 0 | 0.3163279 | 0 | −0.6668104 | X233C241230T25 | 1 |
| 0.17353062 | 0 | 0 | 0 | −0.334448 | X233C241230T26 | 1 |
| 0.04173623 | 0 | 0 | 0 | 2.33924317 | X233C241230T27 | 0 |
| 0.06397567 | 0 | 0.12191589 | 0 | 0.86660576 | X233C241230T28 | 1 |
| 0 | 0 | 0 | 0 | 1.8223788 | X233C241230T29 | 1 |
| 0.19041286 | 0 | 0 | 0.1601886 | 2.98123412 | X233C241230T30 | 1 |
| 0 | 0 | 0 | 0.04282713 | 0.58904182 | X233C241230T31 | 0 |
| 4.4370288 | 1.07526882 | 0 | 0 | 2.91100841 | X233C241230T32 | 0 |
| 2.08576823 | 1.72615302 | 0 | 0.00899038 | 2.27374769 | X233C241230T34 | 1 |
| 0.0419565 | 0.00883295 | 0 | 0 | −0.7837981 | X233C241230T35 | 1 |
| 0.51195787 | 0.05119579 | 0 | 0 | 1.11223212 | X233C241230T36 | 1 |
| 0.16192776 | 0.06449665 | 0 | 0.04802942 | 0.60080964 | X233C241230T37 | 1 |
| 0 | 0 | 0.31758252 | 0 | 1.78700458 | X233C250106T01 | 1 |
| 0.01330254 | 0 | 0 | 0.02660507 | 2.99741648 | X233C250106T11 | 0 |
| 0 | 0 | 0.14913918 | 0 | 0.71656997 | X233C250106T12 | 1 |
| 0 | 0.00128711 | 0 | 0.20851165 | 0.41685455 | X233C250106T14 | 0 |
| 0 | 0 | 0 | 0.22774948 | 0.93528164 | X233C250106T15 | 0 |
| 0 | 0 | 0 | 0 | 2.96023086 | X233C250106T17 | 0 |
| 0 | 0 | 0 | 0 | 0.82279023 | X233C250106T19 | 1 |
| 0 | 0 | 0.38203894 | 0 | −1.0507663 | X233C250106T34 | 1 |
| 0.668037 | 0 | 0 | 0 | 3.0095247 | X233C250106T37 | 1 |
| 4.83676477 | 1.47603948 | 0 | 0 | 2.52431265 | X233C250106T39 | 1 |
| 0 | 0 | 0.52665079 | 0 | −0.1567249 | X233C250106T41 | 0 |
| 4.31391277 | 2.14899714 | 0 | 0 | 2.55628906 | X233C250106T44 | 1 |

### 6.7 SEQUENCES

| SEQ | | Green Genes 2 | |
|---|---|---|---|
| ID NO: | Notes | Accession No: | Sequences |
| 1 | Forward Primer | NA | TAATTGTGTGCCAGCmGCCGCGGTAA |
| | | | m is A or C (see WIPO Standards ST.26 Annex I |
| | | | Section 1) |
| 2 | Reverse Primer | NA | TCAGCCGGACTAChvGGGTwTCTAAT |
| | | | h is A or C or T; v is A or C or G; w is A or T |
| | | | (see WIPO Standards ST.26 Annex I Section 1) |
| 3 | Staphylococcus | L36472 | GCCAGCCGCCGCGGTAATACGTAGGTGGCAAGC |
| | sp. 1, | | GTTATCCGGAATTATTGGGCGTAAAGCGCGCGTA |
| | Staphylococcus | | GGCGGTTTTTTAAGTCTGATGTGAAAGCCCACGG |
| | aureus | | CTCAACCGTGGAGGGTCATTGGAAACTGGAAAAC |
| | (16S rRNA V4 | | TTGAGTG |
| | region) | | |
| 4 | Fenollaria sp. 1, | HM587321 | GCCAGCAGCCGCGGTAATACGTAAGGGGCGAGC |
| | Fenollaria | | GTTGTCCGGAATTATTGGGCGTAAAGAGTGCGTA |
| | massiliensis | | GGCGGCAAATTAAGTCAGATGTGAAAACTAAGG |
| | (16S rRNA V4 | | GCTCAACCCATAGATTGCATCTGAAACTGATATG |
| | region) | | CTTGAGTC |
| 5 | Priestia sp. 1, | RS-GCF- | GCCAGCAGCCGCGGTAATACGTAGGTGGCAAGC |
| | Priestia | 003075295.1- | GTTATCCGGAATTATTGGGCGTAAAGCGCGCGCA |
| | megaterium | NZ- | GGCGGTTTCTTAAGTCTGATGTGAAAGCCCACGG |
| | (16S rRNA V4 | QDFP01000003.01 | CTCAACCGTGGAGGGTCATTGGAAACTGGGGAAC |
| | region) | | TTGAGT |
| 6 | Coprococcus | G000210555 | GCCAGCAGCCGCGGTAATACGAAGGAGGCAAGC |
| | sp. 1, | | GAAGAGCGGAGGTCTTGAGCGTCAATCTCTAGCA |
| | Coprococcus | | GCCGGGTCCCAAAAACGGAAAAGAAAACCTGAG |
| | catus | | GCGAAAACGGAGAAAGGGAACAGAAAATGGTGG |
| | (16S rRNA V4 | | ACATGAGTG |
| | region) | | |
| 7 | Butyricimonas | G001915615 | GCCAGCAGCCGCGGTAATACTTATTTTTCCCTCTT |
| | sp. 1, | | TTTCCTTCTTTCTTTTTCTTCCCTCTCTCTCCTTCTT |
| | Butyricimonas | | CCTCCTCCTTCTTCTTTTCCCTCCCTCTTCTTCCCC |
| | faecihominis | | TCTTCCCTTCCTCTTCCCCTTTTTTTCTTTCTTT |
| | (16S rRNA V4 | | |
| | region) | | |
| 8 | Anaeroglobus | AF338413 | GCCAGCAGCCGCGGTAATACGTAGGTGGCAAGC |
| | sp. 1, | | GTTGTCCGGAATGATTGGGCGTAAAGGGCGCGCA |
| | Anaeroglobus | | GGCGGCTGTGTAAGTCTGTCTAGAAAGTGCGGGG |
| | geminatus | | CTAAACCCCGTGAGAGGATGGAAACTGGACAGC |
| | (16S rRNA V4 | | TGAGAGTG |
| | region) | | |
| 9 | Anaerococcus | Y07841 | GCCAGCAGCCGCGGTAATACGTAAGGACCGAGC |
| | sp. 1, | | GTTGTCCGGAATCATTGGGCGTAAAGGGTACGTA |
| | Anaerococcus | | GGCGGGTCATTAAGTTAGAAGTCAAAGGCTATAG |
| | Octavius | | CTCAACTATAGTAAGCTTCTAAAACTGGAGACCT |
| | (16S rRNA V4 | | TGAGTAA |
| | region) | | |
| 10 | Prevotella sp. 1, | AB547677 | GCCAGCAGCCGCGGTAATACGGAAGGTCCGGGC |
| | Prevotella | | GTTATCCGGATTTATTGGGTTTAAAGGGAGTGTA |
| | corporis | | GGCGGCCTGTTAAGCGTGTTGTGAAATGTAGATG |
| | (16S rRNA V4 | | CTCAACATCTGAACTGCAGCGCGAACTGGCTGGC |
| | region) | | TTGAGTA |
| 11 | Varibaculum | JQ780830 | GCCAGCAGCCGCGGTAATACGTAGGGCGCGAGC |
| | sp. 1, | | GTTGTCCGGAATTATTGGGCGTAAAGGGCTTGTA |
| | Varibaculum | | GGTGGCTGGTTGCGTCTGTCGTGAAAGCTCATGG |
| | anthropi | | CTTAACTGTGGGTTTGCGGTGGGTACGGGCTGGC |
| | (16S rRNA V4 | | TTGAGTG |
| | region) | | |
| 12 | Corynebacterium | X81909 | GCCAGCAGCCGCGGTAATACGTAGGGTGCGAGC |
| | sp. 1, | | GTTGTCCGGATTTACTGGGCGTAAAGAGCTCGTA |
| | Corynebacterium | | GGTGGTTTGTCGCGTCGTCTGTGAAATTCCGGGG |
| | urealyticum | | CTTAACTCCGGGCGTGCAGGCGATACGGGCATAA |
| | (16S rRNA V4 | | CTTGAGT |
| | region) | | |
| 13 | Thalassobacillus | EU817571 | GCCAGCCGCCGCGGTAATACGTAGGTGGCAAGC |
| | sp. 1, | | GTTATCCGGAATTATTGGGCGTAAAGCGCGCGCA |
| | Thalassobacillus | | GGCGGTTTCTTAAGTCTGATGTGAAAGCCCCCGG |
| | hwangdonensis | | CTTAACCGGGGAGGGTCATTGGAAACTGGGGAA |
| | (16S rRNA V4 | | CTTGAGTA |
| | region) | | |
| 14 | Corynebacterium | RS-GCF- | GCCAGCAGCCGCGGTAATACGTAGGGTGCGAGC |
| | sp. 2, | 013408445.1- | GTTGTCCGGAATTACTGGGCGTAAAGGGCTCGTA |
| | Corynebacterium | NZ- | GGTGGTTTGTCGCGTCGTCTGTGAAATTCCGGGG |
| | tuberculostearicum | JACBZL010000001.1--4 | CTTAACTCCGGGCGTGCAGGCGATACGGGCATAA |
| | (16S rRNA V4 | | CTTGAGT |
| | region) | | |
| 15 | Staphylococcus | G000934465 | GCCAGCAGCCGCGGTAATACGTAGGTGGCAAGC |
| | sp. 2, | | GTTATCCGGAATTATTGGGCGTAAAGCGCGCGTA |
| | Staphylococcus | | GGCGGTTTTTTAAGTCTGATGTGAAAGCCCACGG |
| | intermedius | | CTCAACCGTGGAGGGTCATTGGAAACTGGAAAAC |
| | (16S rRNA V4 | | TTGAGTG |
| | region) | | |
| 16 | Finegoldia sp. 1, | RS-GCF- | GCCAGCAGCCGCGGTAATACGTATGGAGCGAGC |
| | Finegoldia | 002243155.1- | GTTGTCCGGAATTATTGGGCGTAAAGGGTACGCA |
| | magna | NZ- | GGCGGTTTAATAAGTCGAATGTTAAAGATCGGGG |
| | (16S rRNA V4 | NDYH01000035.1 | CTCAACCCCGTAAAGCATTGGAAACTGATAAACT |
| | region) | | TGAGTAG |
| 17 | Mobiluncus sp. 1, | AJ427624 | GCCAGCAGCCGCGGTAATACGTAGGGCGCGAGC |
| | Mobiluncus | | GTTGTCCGGATTTATTGGGCGTAAAGAGCTCGTA |
| | curtisii | | GGTGGTTCGTCGCGTCTGTCGTGAAAGCCAGCAG |
| | (16S rRNA V4 | | CTTAACTGTTGGTCTGCGGTGGGTACGGGCGGGC |
| | region) | | TTGAGTG |
| 18 | Cutibacterium | KM507346 | GCCAGCCGCCGCGGTGATACGTAGGGTGCGAGC |
| | sp. 1, | | GTTGTCCGGATTTATTGGGCGTAAAGGGCTCGTA |
| | Cutibacterium | | GGCGGTTGATCGCGTCGGAAGTGAAATCTTGGGG |
| | namnetense | | CTTAACCCTGAGCGTGCTTTCGATACGGGTTGAC |
| | (16S rRNA V4 | | TTGAGGA |
| | region) | | |
| 19 | Peptoniphilus | G000183565 | GCCAGCAGCCGCGGTAATACGTAGGGGGCTAGC |
| | sp. 1, | | GTTGTCCGGAATCACTGGGCGTAAAGGGTTCGCA |
| | Peptoniphilus | | GGCGGAAATGCAAGTCAGGTGTAAAAGGCAGTA |
| | harei | | GCTTAACTACTGTAAGCATTTGAAACTGCATATC |
| | (16S rRNA V4 | | TTGAGAAG |
| | region) | | |
| 20 | Priestia sp. 2, | RS-GCF- | GCCAGCAGCCGCGGTAATACGTAGGTGGCAAGC |
| | Priestia | 017743055.1- | GTTATCCGGAATTATTGGGCGTAAAGCGCGCGCA |
| | aryabhattai | NZ- | GGCGGTTTCTTAAGTCTGATGTGAAAGCCCACGG |
| | (16S rRNA V4 | CP072473.1 | CTCAACCGTGGAGGGTCATTGGAAACTGGGGGAC |
| | region) | | TTGAGTA |
| 21 | Veillonella sp. 1, | AF473836 | GCCAGCAGCCGCGGTAATACGTAGGTGGCAAGC |
| | Veillonella | | GTTGTCCGGAATTATTGGGCGTAAAGCGCGCGCA |
| | atypica | | GGCGGACTAGCCAGTCAGTCTTAAAAGTTCGGGG |
| | (16S rRNA V4 | | CTTAACCCCGTGATGGGATTGAAACTACTAGTCT |
| | region) | | AGAGTAT |
| 22 | Prevotella sp. 2, | AB547706 | GCCAGCAGCCGCGGTAATACGGAAGGTCCGGGC |
| | Prevotella | | GTTATCCGGATTTATTGGGTTTAAAGGGAGCGTA |
| | timonensis | | GGCTGTCTATTAAGCGTGTTGTGAAATTTACCGG |
| | (16S rRNA V4 | | CTCAACCGGTGGCTTGCAGCGCGAACTGGTCGAC |
| | region) | | TTGAGTA |
| 23 | Prevotella sp. 3, | MJ006-1- | GCCAGCAGCCGCGGTAATACGGAAGGTTCGGGC |
| | Prevotella bivia | barcode26- | GTTATCCGGATTTATTGGGTTTAAAGGGAGCGTA |
| | (16S rRNA V4 | umi141087b | GGCCGTTTGGTAAGCGTGTTGTGAAATGTAGGAG |
| | region) | ins-ubs-4 | CTCAACTTCTAGATTGCAGCGCGAACTGTCAGAC |
| | | | TTGAGTG |
| 24 | Gardnerella | RS-GCF- | GCCAGCAGCCGCGGTAATACGTAGGGCGCAAGC |
| | sp. 1, | 014857145.1- | GTTATCCGGAATTATTGGGCGTAAAGAGCTTGTA |
| | Gardnerella | NZ- | GGCGGTTCGTCGCGTCTGGTGTGAAAGCCCATCG |
| | vaginalis | JACZFD010000044.1 | CTTAACGGTGGGTTTGCGCCGGGTACGGGGGGC |
| | (16S rRNA V4 | | TAGAGTG |
| | region) | | |
| 25 | Ureaplasma | RS-GCF- | GCCAGCAGCCGCGGTAATACATAGGATGCAAGC |
| | sp. 1, | 000169915.1- | GTTATCCGGATTTACTGGGCGTAAAACGAGCGCA |
| | Ureaplasma | NZ- | GGCGGGTTTGTAAGTTTTGTATTAAATCTAGATG |
| | urealyticum | AAZR01000010.1 | CTTAACGTCTAGCTGTATCAAAAACTGTAAACCT |
| | (16S rRNA V4 | | AGAGTGT |
| | region) | | |
| 26 | Niallia sp. 1, | MJ031-1- | GCCAGCCGCCGCGGTAATACGTAGGTGGCAAGC |
| | Niallia oryzisoli | barcode30- | GTTATCCGGAATTATTGGGCGTAAAGCGCGCGCA |
| | (16S rRNA V4 | umi33608bi | GGCGGTTTTTTAAGTCTGATGTGAAAGCCCACGG |
| | region) | ns-ubs-8 | CTTAACCGTGGAGGGTCATTGGAAACTGGGGGAC |
| | | | TTGAGTA |
| 27 | Murdochiella | EU483153 | GCCAGCAGCCGCGGTAATACGTAGGGGGCGAGC |
| | sp. 1, | | GTTGTTCGGAATTATTGGGCGTAAAGGGTACGTA |
| | Murdochiella | | GGCGGTTTGTTAAGTTTGGCGTTAAATCACGGGG |
| | asaccharolytica | | CTCAACCCCGTTCAGCGTTGAAAACTGGCAAACT |
| | (16S rRNA V4 | | TGAGTAG |
| | region) | | |
| 28 | Lactobacillus | MJ006-2- | GCCAGCAGCCGCGGTAATACGTAGGTGGCAAGC |
| | sp. 1, | barcode51- | GTTGTCCGGATTTATTGGGCGTAAAGCGAGTGCA |
| | Lactobacillus | umi102309b | GGCGGCTCGATAAGTCTGATGTGAAAGCCTTCGG |
| | iners | ins-ubs-5 | CTCAACCGGAGAATTGCATCAGAAACTGTCGAGC |
| | (16S rRNA V4 | | TTGAGTA |
| | region) | | |
| 29 | Lactobacillus | G000466805 | GCCAGCAGCCGCGGTAATACGTAGGTGGCAAGC |
| | sp. 2, | | GTTGTCCGGATTTATTGGGCGTAAAGCGAGCGCA |
| | Lactobacillus | | GGCGGATTGATAAGTCTGATGTGAAAGCCTTCGG |
| | jensenii | | CTCAACCGAAGAACTGCATCAGAAACTGTCAATC |
| | (16S rRNA V4 | | TTGAGTG |
| | region) | | |
| 30 | Lawsonella sp. 1, | JX877776 | GCCAGCAGCCGCGGTAATACGTAGGGTGCGAGC |
| | Lawsonella | | GTTGTCCGGAATTACTGGGCGTAAAGAGCTCGTA |
| | clevelandensis | | GGCGGTTTGTCACGTCGTCTGTGAAATCCTAGGG |
| | (16S rRNA V4 | | CTTAACCCTGGACGTGCAGGCGATACGGGCTGAC |
| | region) | | TTGAGTA |
| 31 | Corynebacterium | CP001620 | GCCAGCAGCCGCGGTAATACGTAGGGTGCGAGC |
| | sp. 3, | | GTTGTCCGGAATTACTGGGCGTAAAGAGCTCGTA |
| | Corynebacterium | | GGTGGTCTGTCGCGTCATTTGTGAAAGCCCGGGG |
| | kroppenstedtii | | CTTAACTCCGGGTTGGCAGGTGATACGGGCATGA |
| | (16S rRNA V4 | | CTGGAGT |
| | region) | | |
| 32 | Lactobacillus | GB-GCA- | GCCAGCAGCCGCGGTAATACGTAGGTGGCAAGC |
| | sp. 3, | 000466885.2- | GTTGTCCGGATTTATTGGGCGTAAAGCGAGCGCA |
| | Lactobacillus | AVFH02000268.1 | GGCGGAAGAATAAGTCTGATGTGAAAGCCCTCG |
| | crispatus | | GCTTAACCGAGGAACTGCATCGGAAACTGTTTTT |
| | (16S rRNA V4 | | CTTGAGTG |
| | region) | | |
| 33 | Dialister sp. 1, | AY850119 | GCCAGCAGCCGCGGTAATACGTAGGTGGCAAGC |
| | Dialister | | GTTGTCCGGAATTATTGGGCGTAAAGCGCGCGCA |
| | hominis | | GGCGGCTTCCTAAGTCCATCTTAAAAGTGCGGGG |
| | (16S rRNA V4 | | CTTAACCCCGTGATGGGATGGAAACTGGGAAGCT |
| | region) | | GGAGTAT |
| 34 | Lactobacillus | G000159435 | GCCAGCAGCCGCGGTAATACGTAGGTGGCAAGC |
| | sp. 4, | | GTTATCCGGATTTATTGGGCGTAAAGCGAGCGCA |
| | Lactobacillus | | GGCGGTTGCTTAGGTCTGATGTGAAAGCCTTCGG |
| | vaginalis | | CTTAACCGAAGAAGGGCATCGGAAACCGGGCGA |
| | (16S rRNA V4 | | CTTGAGTG |
| | region) | | |
| 35 | Ureaplasma | AF073456 | GCCAGCAGCCGCGGTAATACATAGGATGCAAGC |
| | sp. 2, | | GTTATCCGGATTTACTGGGCGTAAAACGAGCGCA |
| | Ureaplasma | | GGCGGGTTTGTAAGTTTGGTATTAAATCTAGATG |
| | parvum | | CTTAACGTCTAGCTGTATCAAAAACTGTAAACCT |
| | (16S rRNA V4 | | AGAGTGT |
| | region) | | |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Unless the context indicates otherwise, it is specifically intended that the various features described herein can be used in any combination.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

1-60. (canceled)

61. A method for characterizing a microbiome to assess a likelihood of endometriosis in a subject, comprising:
(a) obtaining a dataset representing a plurality of nucleic acid sequences derived from a sample obtained from the subject;
(b) quantifying, from the dataset, a relative abundance of a panel of bacterial taxa;
(c) calculating a Functional Dysbiosis Score (FDS) for the sample based on a relative abundance of Lactobacillus spp. and a cumulative relative abundance of a plurality of pathogenic taxa; and
(d) processing the relative abundance of the panel of bacterial taxa and the FDS using a trained machine learning classifier to generate a classification output indicating the presence or absence of endometriosis.

62. The method of claim 61, wherein the sample is obtained during the proliferative phase of a menstrual cycle; optionally wherein the method further comprises measuring a serum progesterone level of the subject, wherein the proliferative phase is confirmed if the serum progesterone level is not above a reference level; optionally wherein the reference level is 1.08 ng/mL.

63. The method of claim 62, wherein the panel of bacterial taxa comprises at least 1, 2, 3, 4, 5, 6, 7, 8, 9, 10, 11, 12, 13, 14, 15, 16, or 17 taxa selected from the group consisting of: Fenollaria, Anaeroglobus, Anaerococcus, Coprococcus, Prevotella, Varibaculum, Corynebacterium, Thalassobacillus, Staphylococcus, Priestia, Butyricimonas, Finegoldia, Mobiluncus, Cutibacterium, Peptoniphilus, Veillonella, and Gardnerella; optionally wherein the panel comprises at least one of Coprococcus and Butyricimonas; and at least one of Gardnerella and Prevotella.

64. The method of claim 63, wherein the panel of bacterial taxa comprises at least 1, 2, 3, 4, 5, 6, 7, 8, 9, 10, 11, 12, 13, 14, 15, 16, 17, 18, 19, 20, 21 or 22 taxa (1) selected from the group consisting of: Staphylococcus aureus, Fenollaria massiliensis, Priestia megaterium, Coprococcus catus, Butyricimonas faecihominis, Anaeroglobus geminatus, Anaerococcus octavius, Prevotella corporis, Varibaculum anthropi, Corynebacterium urealyticum, Thalassobacillus hwangdonensis, Corynebacterium tuberculostearicum, Staphylococcus intermedius, Finegoldia magna, Mobiluncus curtisii, Cutibacterium namnetense, Peptoniphilus harei, Priestia aryabhattai, Veillonella atypica, Prevotella timonensis, Prevotella bivia, and Gardnerella vaginalis; or
(2) selected from the group consisting of the taxa listed below, wherein each taxon is identified by the V4 region of a 16S rRNA gene sequence having at least 97% identity to the corresponding SEQ ID NO indicated in parentheses: (i) Staphylococcus sp.1 (SEQ ID NO:3); (ii) Fenollaria sp.1 (SEQ ID NO:4); (iii) Priestia sp.1 (SEQ ID NO:5); (iv) Coprococcus sp.1 (SEQ ID NO:6); (v) Butyricimonas sp.1 (SEQ ID NO:7); (vi) Anaeroglobus sp.1 (SEQ ID NO:8); (vii) Anaerococcus sp.1 (SEQ ID NO: 9); (viii) Prevotella sp.1 (SEQ ID NO: 10); (ix) Varibaculum sp.1 (SEQ ID NO: 11); (x) Corynebacterium sp.1 (SEQ ID NO: 12); (xi) Thalassobacillus sp.1 (SEQ ID NO: 13); (xii) Corynebacterium sp.2 (SEQ ID NO: 14); (xiii) Staphylococcus sp.2 (SEQ ID NO:15); (xiv) Finegoldia sp.1 (SEQ ID NO: 16); (xv) Mobiluncus sp.1 (SEQ ID NO: 17); (xvi) Cutibacterium sp.1 (SEQ ID NO:18); (xvii) Peptoniphilus sp.1 (SEQ ID NO:19); (xviii) Priestia sp.2 (SEQ ID NO:20); (xix) Veillonella sp.1 (SEQ ID NO:21); (xx) Prevotella sp.2 (SEQ ID NO:22); (xxi) Prevotella sp.3 (SEQ ID NO:23); and (xxii) Gardnerella sp.1 (SEQ ID NO:24);
optionally wherein the panel comprises (i) at least one of Coprococcus catus and Butyricimonas faecihominis; and at least one of Gardnerella vaginalis, Prevotella corporis, Prevotella timonensis, and Prevotella bivia; or (ii) at least one of Coprococcus sp.1 (SEQ ID NO:6) and Butyricimonas sp.1 (SEQ ID NO:7); and at least one of Gardnerella sp.1 (SEQ ID NO:24), Prevotella sp.1 (SEQ ID NO:10), Prevotella sp.2 (SEQ ID NO:22), and Prevotella sp.3 (SEQ ID NO:23).

65. The method of claim 61, wherein the sample is obtained during the secretory phase of a menstrual cycle; optionally wherein the method further comprises measuring a serum progesterone level of the subject, wherein the secretory phase is confirmed if the serum progesterone level is above a reference level; optionally wherein the reference level is 1.08 ng/mL.

66. The method of claim 65, wherein the panel of bacterial taxa comprises at least 1, 2, 3, 4, 5, 6, 7, 8, 9 or 10 taxa selected from the group consisting of: Ureaplasma, Niallia, Murdochiella, Gardnerella, Lactobacillus, Lawsonella, Corynebacterium, Priestia, Finegoldia, and Dialister.

67. The method of claim 66, wherein the panel of bacterial taxa comprises at least 1, 2, 3, 4, 5, 6, 7, 8, 9, 10, 11, 12, 13, or 14 taxa
(1) selected from the group consisting of Ureaplasma urealyticum, Niallia oryzisoli, Murdochiella asaccharolytica, Gardnerella vaginalis, Lactobacillus iners, Lactobacillus jensenii, Lawsonella clevelandensis, Corynebacterium kroppenstedtii, Priestia megaterium, Lactobacillus crispatus, Finegoldia magna, Dialister hominis, Lactobacillus vaginalis, and Ureaplasma parvum; or
(2) selected from the group consisting of the taxa listed below, wherein each taxon is identified by the V4 region of a 16S rRNA gene sequence having at least 97% identity to the corresponding SEQ ID NO indicated in parentheses: Ureaplasma sp.1 (SEQ ID NO:25), Niallia sp.1 (SEQ ID NO:26), Murdochiella sp.1 (SEQ ID NO:27), Gardnerella sp.1 (SEQ ID NO:24), Lactobacillus sp.1 (SEQ ID NO:28), Lactobacillus sp.2 (SEQ ID NO:29), Lawsonella sp.1 (SEQ ID NO:30), Corynebacterium sp.3 (SEQ ID NO:31), Priestia sp.1 (SEQ ID NO:5), Lactobacillus sp.3 (SEQ ID NO:32), Finegoldia sp.1 (SEQ ID NO: 16), Dialister sp.1 (SEQ ID NO:33), Lactobacillus sp.4 (SEQ ID NO:34), and Ureaplasma sp.2 (SEQ ID NO:35).

68. The method of claim 61, wherein the FDS is calculated by the formula: FDS=0.5×(1−A_{Lacto})+10×Aₚₐₜₕₒ, wherein A_{Lacto} is the relative abundance of Lactobacillus and Aₚₐₜₕₒ is the cumulative relative abundance of the plurality of pathogenic taxa; optionally wherein the pathogenic taxa used to calculate the FDS comprise one or more taxa selected from the group consisting of: Gardnerella, Prevotella, Anaerococcus, Streptococcus, Megasphaera, Mobiluncus, Sneathia, Atopobium, Peptoniphilus, Mycoplasmoides, Ureaplasma, Bacteroides, Peptostreptococcus and Dialister.

69. The method of claim 61, wherein the trained machine learning classifier is a Random Forest classifier; optionally wherein the Random Forest classifier has been trained using repeated random subsampling cross-validation on a training dataset comprising microbiome profiles from subjects with confirmed endometriosis and controls; optionally wherein the training data set is randomly split into 80% for training and 20% for testing in each iteration; optionally wherein the classifier is trained for at least 50 iterations of repeated cross-validation.

70. The method of claim 69, wherein the bacterial taxa of the training dataset are selected by performing a multivariable association analysis; optionally wherein the multivariable association analysis is performed using Microbiome Multivariable Associations with Linear Models (MaAsLin2), optionally controlled for a confounding variable; optionally wherein the confounding variables are age and Body Mass Index (BMI).

71. The method of claim 61, wherein obtaining the dataset comprises: (i) extracting genomic DNA from the sample; (ii) amplifying the V4 region of 16S rRNA genes from the extracted genomic DNA to generate amplicons; and (iii) sequencing the amplicons; optionally wherein the amplifying is performed using a primer set having the nucleotide sequences of SEQ ID NOs:1 and 2; optionally wherein the method further comprises bioinformatically removing sequencing reads mapping to a human reference genome prior to step (b).

72. The method of claim 61, wherein the sample comprises cervicovaginal fluid, vaginal mucus, cervical mucus, blood, vaginal mucosa, interstitial fluid, uterine fluid, cervical secretion, uterine tissue, reproductive cells, cervical cells, endometrial cells, fallopian cells, ovarian cells, or natural flora in a female reproductive tract; optionally wherein the sample comprises endometrial cells, vaginal mucus, uterine tissue, or uterine fluid.

73. The method of claim 61, further comprising measuring a protein biomarker or a miRNA biomarker for endometriosis in the sample.

74. The method of claim 61, wherein the subject has a clinical indicator for endometriosis, wherein the indicator is dysmenorrhea, lower abdominal pain, chronic pelvic pain, deep dyspareunia, dysuria, dyschezia, fatigue, or infertility, or any combination thereof; or wherein the subject is asymptomatic.

75. The method of claim 61, further comprising administering a treatment for endometriosis to the subject; optionally wherein the treatment for endometriosis is pain medication, a hormone therapy, or a surgical procedure, or any combination thereof; optionally wherein the treatment for endometriosis is laparoscopic excision, gonadotropin-releasing hormone (GnRH) agonist or antagonist, oral contraceptive, or progestin, or any combination thereof.

76. A kit for assessing whether a subject has endometriosis, comprising (1) a means for obtaining a dataset representing a plurality of nucleic acid sequences in a sample from the subject, and (2) a non-transitory computer-readable medium storing instructions that, when executed by a processor, cause the processor to:
(i) receive the obtained dataset;
(ii) quantify a relative abundance of a panel of bacterial taxa;
(iii) calculate a FDS for the sample based on a relative abundance of Lactobacillus spp. and a cumulative relative abundance of a plurality of pathogenic taxa; and
(iv) input the relative abundance of the panel of bacterial taxa and FDS into a trained machine learning classifier to generate a classification output indicating the presence or absence of endometriosis.

77. The kit of claim 76, wherein the sample is obtained during the proliferative phase of a menstrual cycle; optionally wherein the panel of bacterial taxa comprises at least 1, 2, 3, 4, 5, 6, 7, 8, 9, 10, 11, 12, 13, 14, 15, 16, or 17 taxa selected from the group consisting of: Fenollaria, Anaeroglobus, Anaerococcus, Coprococcus, Prevotella, Varibaculum, Corynebacterium, Thalassobacillus, Staphylococcus, Priestia, Butyricimonas, Finegoldia, Mobiluncus, Cutibacterium, Peptoniphilus, Veillonella, and Gardnerella; optionally wherein the panel comprises at least one of Coprococcus and Butyricimonas; and at least one of Gardnerella and Prevotella.

78. The kit of claim 77, wherein the panel of bacterial taxa comprises at least 1, 2, 3, 4, 5, 6, 7, 8, 9, 10, 11, 12, 13, 14, 15, 16, 17, 18, 19, 20, 21 or 22 taxa
(1) selected from the group consisting of: Staphylococcus aureus, Fenollaria massiliensis, Priestia megaterium, Coprococcus catus, Butyricimonas faecihominis, Anaeroglobus geminatus, Anaerococcus octavius, Prevotella corporis, Varibaculum anthropi, Corynebacterium urealyticum, Thalassobacillus hwangdonensis, Corynebacterium tuberculostearicum, Staphylococcus intermedius, Finegoldia magna, Mobiluncus curtisii, Cutibacterium namnetense, Peptoniphilus harei, Priestia aryabhattai, Veillonella atypica, Prevotella timonensis, Prevotella bivia, and Gardnerella vaginalis; or
(2) selected from the group consisting of the taxa listed below, wherein each taxon is identified by the V4 region of a 16S rRNA gene sequence having at least 97% identity to the corresponding SEQ ID NO indicated in parentheses: (i) Staphylococcus sp.1 (SEQ ID NO:3); (ii) Fenollaria sp.1 (SEQ ID NO: 4); (iii) Priestia sp.1 (SEQ ID NO:5); (iv) Coprococcus sp.1 (SEQ ID NO:6); (v) Butyricimonas sp.1 (SEQ ID NO:7); (vi) Anaeroglobus sp.1 (SEQ ID NO:8); (vii) Anaerococcus sp.1 (SEQ ID NO:9); (viii) Prevotella sp.1 (SEQ ID NO: 10); (ix) Varibaculum sp.1 (SEQ ID NO: 11); (x) Corynebacterium sp.1 (SEQ ID NO: 12); (xi) Thalassobacillus sp.1 (SEQ ID NO: 13); (xii) Corynebacterium sp.2 (SEQ ID NO: 14); (xiii) Staphylococcus sp.2 (SEQ ID NO: 15); (xiv) Finegoldia sp.1 (SEQ ID NO: 16); (xv) Mobiluncus sp.1 (SEQ ID NO: 17); (xvi) Cutibacterium sp.1 (SEQ ID NO:18); (xvii) Peptoniphilus sp.1 (SEQ ID NO:19); (xviii) Priestia sp.2 (SEQ ID NO:20); (xix) Veillonella sp.1 (SEQ ID NO:21); (xx) Prevotella sp.2 (SEQ ID NO:22); (xxi) Prevotella sp.3 (SEQ ID NO:23); and (xxii) Gardnerella sp.1 (SEQ ID NO:24);
optionally wherein the panel comprises (i) at least one of Coprococcus catus and Butyricimonas faecihominis; and at least one of Gardnerella vaginalis, Prevotella corporis, Prevotella timonensis, and Prevotella bivia; or (ii) at least one of Coprococcus sp.1 (SEQ ID NO:6) and Butyricimonas sp.1 (SEQ ID NO:7); and at least one of Gardnerella sp.1 (SEQ ID NO:24), Prevotella sp.1 (SEQ ID NO:10), Prevotella sp.2 (SEQ ID NO:22), and Prevotella sp.3 (SEQ ID NO:23).

79. The kit of claim 76, wherein the sample is obtained during the secretory phase of a menstrual cycle; optionally wherein the panel of bacterial taxa comprises at least 1, 2, 3, 4, 5, 6, 7, 8, 9 or 10 taxa selected from the group consisting of: Ureaplasma, Niallia, Murdochiella, Gardnerella, Lactobacillus, Lawsonella, Corynebacterium, Priestia, Finegoldia, and Dialister.

80. The kit of claim 79, wherein the panel of bacterial taxa comprises at least 1, 2, 3, 4, 5, 6, 7, 8, 9, 10, 11, 12, 13, or 14 taxa
(1) selected from the group consisting of Ureaplasma urealyticum, Niallia oryzisoli, Murdochiella asaccharolytica, Gardnerella vaginalis, Lactobacillus iners, Lactobacillus jensenii, Lawsonella clevelandensis, Corynebacterium kroppenstedtii, Priestia megaterium, Lactobacillus crispatus, Finegoldia magna, Dialister hominis, Lactobacillus vaginalis, and Ureaplasma parvum; or
(2) selected from the group consisting of the taxa listed below, wherein each taxon is identified by the V4 region of a 16S rRNA gene sequence having at least 97% identity to the corresponding SEQ ID NO indicated in parentheses: Ureaplasma sp.1 (SEQ ID NO:25), Niallia sp.1 (SEQ ID NO:26), Murdochiella sp.1 (SEQ ID NO:27), Gardnerella sp.1 (SEQ ID NO:24), Lactobacillus sp.1 (SEQ ID NO:28), Lactobacillus sp.2 (SEQ ID NO:29), Lawsonella sp.1 (SEQ ID NO:30), Corynebacterium sp.3 (SEQ ID NO:31), Priestia sp.1 (SEQ ID NO:5), Lactobacillus sp.3 (SEQ ID NO:32), Finegoldia sp.1 (SEQ ID NO:16), Dialister sp.1 (SEQ ID NO:33), Lactobacillus sp.4 (SEQ ID NO:34), and Ureaplasma sp.2 (SEQ ID NO:35).

81. The kit of claim 76, wherein the FDS is calculated by the formula: FDS=0.5×(1−A_{Lacto})+10×Aₚₐₜₕₒ, wherein A_{Lacto} is the relative abundance of Lactobacillus and Aₚₐₜₕₒ is the cumulative relative abundance of the plurality of pathogenic taxa; optionally wherein the pathogenic taxa used to calculate the FDS comprise one or more genera selected from the group consisting of: Gardnerella, Prevotella, Anaerococcus, Streptococcus, Megasphaera, Mobiluncus, Sneathia, Atopobium, Peptoniphilus, Mycoplasmoides, Ureaplasma, Bacteroides, Peptostreptococcus and Dialister.

82. The kit of claim 76, wherein the trained machine learning classifier is a Random Forest classifier; optionally wherein the Random Forest classifier has been trained using repeated random subsampling cross-validation on a training dataset comprising microbiome profiles from subjects with confirmed endometriosis and controls; optionally wherein the training dataset is randomly split into 80% for training and 20% for testing in each iteration; optionally wherein the classifier is trained over 50 iterations of repeated cross-validation.

83. The kit of claim 76, wherein the means for obtaining a dataset comprises a primer set configured to amplify the V4 region of bacterial 16S rRNA; optionally wherein the primers have nucleotide sequences of SEQ ID NOs:1 and 2.

84. The kit of claim 76, wherein the kit further comprises a container for sample collection; optionally wherein the sample comprises cervicovaginal fluid, vaginal mucus, cervical mucus, blood, vaginal mucosa, interstitial fluid, cervical secretion, uterine tissue, uterine fluid, reproductive cells, cervical cells, endometrial cells, fallopian cells, ovarian cells, or natural flora in a female reproductive tract; optionally wherein the sample comprises endometrial cells, vaginal mucus, uterine tissue, or uterine fluid.


SEQUENCE LISTING (35 sequences)

SEQ ID NO: 1 - DNA, 26 bp, synthetic construct
  source 1..26: mol_type = other DNA
  misc_feature 1..26: note = Forward Primer
taattgtgtgccagcmgccgcggtaa

SEQ ID NO: 2 - DNA, 26 bp, synthetic construct
  source 1..26: mol_type = other DNA
  misc_feature 1..26: note = Reverse Primer
tcagccggactachvgggtwtctaat

SEQ ID NO: 3 - DNA, 142 bp, Staphylococcus aureus
  misc_feature 1..142: note = Staphylococcus sp.1, Staphylococcus aureus (16S rRNA V4 region)
  source 1..142: mol_type = other DNA
gccagccgccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtg

SEQ ID NO: 4 - DNA, 142 bp, Fenollaria massiliensis
  misc_feature 1..142: note = Fenollaria sp.1, Fenollaria massiliensis (16S rRNA V4 region)
  source 1..142: mol_type = other DNA
gccagcagccgcggtaatacgtaaggggcgagcgttgtccggaattattgggcgtaaagagtgcgtaggcggcaaattaagtcagatgtgaaaactaagggctcaacccatagattgcatctgaaactgatatgcttgagtc

SEQ ID NO: 5 - DNA, 141 bp, Priestia megaterium
  misc_feature 1..141: note = Priestia sp.1, Priestia megaterium (16S rRNA V4 region)
  source 1..141: mol_type = other DNA
gccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagt

SEQ ID NO: 6 - DNA, 142 bp, Coprococcus catus
  misc_feature 1..142: note = Coprococcus sp.1, Coprococcus catus (16S rRNA V4 region)
  source 1..142: mol_type = other DNA
gccagcagccgcggtaatacgaaggaggcaagcgaagagcggaggtcttgagcgtcaatctctagcagccgggtcccaaaaacggaaaagaaaacctgaggcgaaaacggagaaagggaacagaaaatggtggacatgagtg

SEQ ID NO: 7 - DNA, 142 bp, Butyricimonas faecihominis
  misc_feature 1..142: note = Butyricimonas sp.1, Butyricimonas faecihominis (16S rRNA V4 region)
  source 1..142: mol_type = other DNA
gccagcagccgcggtaatacttatttttccctctttttccttctttctttttcttccctctctctccttcttcctcctccttcttcttttccctccctcttcttcccctcttcccttcctcttcccctttttttctttcttt

SEQ ID NO: 8 - DNA, 142 bp, Anaeroglobus geminatus
  misc_feature 1..142: note = Anaeroglobus sp.1, Anaeroglobus geminatus (16S rRNA V4 region)
  source 1..142: mol_type = other DNA
gccagcagccgcggtaatacgtaggtggcaagcgttgtccggaatgattgggcgtaaagggcgcgcaggcggctgtgtaagtctgtctagaaagtgcggggctaaaccccgtgagaggatggaaactggacagctgagagtg

SEQ ID NO: 9 - DNA, 142 bp, Anaerococcus octavius
  misc_feature 1..142: note = Anaerococcus sp.1, Anaerococcus Octavius (16S rRNA V4 region)
  source 1..142: mol_type = other DNA
gccagcagccgcggtaatacgtaaggaccgagcgttgtccggaatcattgggcgtaaagggtacgtaggcgggtcattaagttagaagtcaaaggctatagctcaactatagtaagcttctaaaactggagaccttgagtaa

SEQ ID NO: 10 - DNA, 142 bp, Prevotella corporis
  misc_feature 1..142: note = Prevotella sp.1, Prevotella corporis (16S rRNA V4 region)
  source 1..142: mol_type = other DNA
gccagcagccgcggtaatacggaaggtccgggcgttatccggatttattgggtttaaagggagtgtaggcggcctgttaagcgtgttgtgaaatgtagatgctcaacatctgaactgcagcgcgaactggctggcttgagta

SEQ ID NO: 11 - DNA, 142 bp, Varibaculum anthropi
  misc_feature 1..142: note = Varibaculum sp.1, Varibaculum anthropi (16S rRNA V4 region)
  source 1..142: mol_type = other DNA
gccagcagccgcggtaatacgtagggcgcgagcgttgtccggaattattgggcgtaaagggcttgtaggtggctggttgcgtctgtcgtgaaagctcatggcttaactgtgggtttgcggtgggtacgggctggcttgagtg

SEQ ID NO: 12 - DNA, 142 bp, Corynebacterium urealyticum
  misc_feature 1..142: note = Corynebacterium sp.1, Corynebacterium urealyticum (16S rRNA V4 region)
  source 1..142: mol_type = other DNA
gccagcagccgcggtaatacgtagggtgcgagcgttgtccggatttactgggcgtaaagagctcgtaggtggtttgtcgcgtcgtctgtgaaattccggggcttaactccgggcgtgcaggcgatacgggcataacttgagt

SEQ ID NO: 13 - DNA, 142 bp, Thalassobacillus hwangdonensis
  misc_feature 1..142: note = Thalassobacillus sp.1, Thalassobacillus hwangdonensis (16S rRNA V4 region)
  source 1..142: mol_type = other DNA
gccagccgccgcggtaatacgtaggtggcaagcgttatccggaattattgggcgtaaagcgcgcgcaggcggtttcttaagtctgatgtgaaagcccccggcttaaccggggagggtcattggaaactggggaacttgagta

SEQ ID NO: 14 - DNA, 142 bp, Corynebacterium tuberculostearicum
  misc_feature 1..142: note = Corynebacterium sp.2, Corynebacterium tuberculostearicum (16S rRNA V4 region)
  source 1..142: mol_type = other DNA
gccagcagccgcggtaatacgtagggtgcgagcgttgtccggaattactgggcgtaaagggctcgtaggtggtttgtcgcgtcgtctgtgaaattccggggcttaactccgggcgtgcaggcgatacgggcataacttgagt

SEQ ID NO: 15 - DNA, 142 bp, Staphylococcus intermedius
  misc_feature 1..142: note = Staphylococcus sp.2, Staphylococcus intermedius (16S rRNA V4 region)
  source 1..142: mol_type = other DNA
gccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtg

SEQ ID NO: 16 - DNA, 142 bp, Finegoldia magna
  misc_feature 1..142: note = Finegoldia sp.1, Finegoldia magna (16S rRNA V4 region)
  source 1..142: mol_type = other DNA
gccagcagccgcggtaatacgtatggagcgagcgttgtccggaattattgggcgtaaagggtacgcaggcggtttaataagtcgaatgttaaagatcggggctcaaccccgtaaagcattggaaactgataaacttgagtag

SEQ ID NO: 17 - DNA, 142 bp, Mobiluncus curtisii
  misc_feature 1..142: note = Mobiluncus sp.1, Mobiluncus curtisii (16S rRNA V4 region)
  source 1..142: mol_type = other DNA
gccagcagccgcggtaatacgtagggcgcgagcgttgtccggatttattgggcgtaaagagctcgtaggtggttcgtcgcgtctgtcgtgaaagccagcagcttaactgttggtctgcggtgggtacgggcgggcttgagtg

SEQ ID NO: 18 - DNA, 142 bp, Cutibacterium namnetense
  misc_feature 1..142: note = Cutibacterium sp.1, Cutibacterium namnetense (16S rRNA V4 region)
  source 1..142: mol_type = other DNA
gccagccgccgcggtgatacgtagggtgcgagcgttgtccggatttattgggcgtaaagggctcgtaggcggttgatcgcgtcggaagtgaaatcttggggcttaaccctgagcgtgctttcgatacgggttgacttgagga

SEQ ID NO: 19 - DNA, 142 bp, Peptoniphilus harei
  misc_feature 1..142: note = Peptoniphilus sp.1, Peptoniphilus harei (16S rRNA V4 region)
  source 1..142: mol_type = other DNA
gccagcagccgcggtaatacgtagggggctagcgttgtccggaatcactgggcgtaaagggttcgcaggcggaaatgcaagtcaggtgtaaaaggcagtagcttaactactgtaagcatttgaaactgcatatcttgagaag

SEQ ID NO: 20 - DNA, 142 bp, Priestia aryabhattai
  misc_feature 1..142: note = Priestia sp.2, Priestia aryabhattai (16S rRNA V4 region)
  source 1..142: mol_type = other DNA
gccagcagccgcggtaatacgtaggtggcaagcgttatccggaattattgggcgtaaagcgcgcgcaggcggtttcttaagtctgatgtgaaagcccacggctcaaccgtggagggtcattggaaactgggggacttgagta

SEQ ID NO: 21 - DNA, 142 bp, Veillonella atypica
  misc_feature 1..142: note = Veillonella sp.1, Veillonella atypica (16S rRNA V4 region)
  source 1..142: mol_type = other DNA
gccagcagccgcggtaatacgtaggtggcaagcgttgtccggaattattgggcgtaaagcgcgcgcaggcggactagccagtcagtcttaaaagttcggggcttaaccccgtgatgggattgaaactactagtctagagtat

SEQ ID NO: 22 - DNA, 142 bp, Prevotella timonensis
  misc_feature 1..142: note = Prevotella sp.2, Prevotella timonensis (16S rRNA V4 region)
  source 1..142: mol_type = other DNA
gccagcagccgcggtaatacggaaggtccgggcgttatccggatttattgggtttaaagggagcgtaggctgtctattaagcgtgttgtgaaatttaccggctcaaccggtggcttgcagcgcgaactggtcgacttgagta

SEQ ID NO: 23 - DNA, 142 bp, Prevotella bivia
  misc_feature 1..142: note = Prevotella sp.3, Prevotella bivia (16S rRNA V4 region)
  source 1..142: mol_type = other DNA
gccagcagccgcggtaatacggaaggttcgggcgttatccggatttattgggtttaaagggagcgtaggccgtttggtaagcgtgttgtgaaatgtaggagctcaacttctagattgcagcgcgaactgtcagacttgagtg

SEQ ID NO: 24 - DNA, 142 bp, Gardnerella vaginalis
  misc_feature 1..142: note = Gardnerella sp.1, Gardnerella vaginalis (16S rRNA V4 region)
  source 1..142: mol_type = other DNA
gccagcagccgcggtaatacgtagggcgcaagcgttatccggaattattgggcgtaaagagcttgtaggcggttcgtcgcgtctggtgtgaaagcccatcgcttaacggtgggtttgcgccgggtacgggcgggctagagtg

SEQ ID NO: 25 - DNA, 142 bp, Ureaplasma urealyticum
  misc_feature 1..142: note = Ureaplasma sp.1, Ureaplasma urealyticum (16S rRNA V4 region)
  source 1..142: mol_type = other DNA
gccagcagccgcggtaatacataggatgcaagcgttatccggatttactgggcgtaaaacgagcgcaggcgggtttgtaagttttgtattaaatctagatgcttaacgtctagctgtatcaaaaactgtaaacctagagtgt

SEQ ID NO: 26 - DNA, 142 bp, Niallia oryzisoli
  misc_feature 1..142: note = Niallia sp.1, Niallia oryzisoli (16S rRNA V4 region)
  source 1..142: mol_type = other DNA
gccagccgccgcggtaatacgtaggtggcaagcgttatccggaattattgggcgtaaagcgcgcgcaggcggttttttaagtctgatgtgaaagcccacggcttaaccgtggagggtcattggaaactgggggacttgagta

SEQ ID NO: 27 - DNA, 142 bp, Murdochiella asaccharolytica
  misc_feature 1..142: note = Murdochiella sp.1, Murdochiella asaccharolytica (16S rRNA V4 region)
  source 1..142: mol_type = other DNA
gccagcagccgcggtaatacgtagggggcgagcgttgttcggaattattgggcgtaaagggtacgtaggcggtttgttaagtttggcgttaaatcacggggctcaaccccgttcagcgttgaaaactggcaaacttgagtag

SEQ ID NO: 28 - DNA, 142 bp, Lactobacillus iners
  misc_feature 1..142: note = Lactobacillus sp.1, Lactobacillus iners (16S rRNA V4 region)
  source 1..142: mol_type = other DNA
gccagcagccgcggtaatacgtaggtggcaagcgttgtccggatttattgggcgtaaagcgagtgcaggcggctcgataagtctgatgtgaaagccttcggctcaaccggagaattgcatcagaaactgtcgagcttgagta

SEQ ID NO: 29 - DNA, 142 bp, Lactobacillus jensenii
  misc_feature 1..142: note = Lactobacillus sp.2, Lactobacillus jensenii (16S rRNA V4 region)
  source 1..142: mol_type = other DNA
gccagcagccgcggtaatacgtaggtggcaagcgttgtccggatttattgggcgtaaagcgagcgcaggcggattgataagtctgatgtgaaagccttcggctcaaccgaagaactgcatcagaaactgtcaatcttgagtg

SEQ ID NO: 30 - DNA, 142 bp, Lawsonella clevelandensis
  misc_feature 1..142: note = Lawsonella sp.1, Lawsonella clevelandensis (16S rRNA V4 region)
  source 1..142: mol_type = other DNA
gccagcagccgcggtaatacgtagggtgcgagcgttgtccggaattactgggcgtaaagagctcgtaggcggtttgtcacgtcgtctgtgaaatcctagggcttaaccctggacgtgcaggcgatacgggctgacttgagta

SEQ ID NO: 31 - DNA, 142 bp, Corynebacterium kroppenstedtii
  misc_feature 1..142: note = Corynebacterium sp.3, Corynebacterium kroppenstedtii (16S rRNA V4 region)
  source 1..142: mol_type = other DNA
gccagcagccgcggtaatacgtagggtgcgagcgttgtccggaattactgggcgtaaagagctcgtaggtggtctgtcgcgtcatttgtgaaagcccggggcttaactccgggttggcaggtgatacgggcatgactggagt

SEQ ID NO: 32 - DNA, 142 bp, Lactobacillus crispatus
  misc_feature 1..142: note = Lactobacillus sp.3, Lactobacillus crispatus (16S rRNA V4 region)
  source 1..142: mol_type = other DNA
gccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtg

SEQ ID NO: 33 - DNA, 142 bp, Dialister hominis
  misc_feature 1..142: note = Dialister sp.1, Dialister hominis (16S rRNA V4 region)
  source 1..142: mol_type = other DNA
gccagcagccgcggtaatacgtaggtggcaagcgttgtccggaattattgggcgtaaagcgcgcgcaggcggcttcctaagtccatcttaaaagtgcggggcttaaccccgtgatgggatggaaactgggaagctggagtat

SEQ ID NO: 34 - DNA, 142 bp, Lactobacillus vaginalis
  misc_feature 1..142: note = Lactobacillus sp.4, Lactobacillus vaginalis (16S rRNA V4 region)
  source 1..142: mol_type = other DNA
gccagcagccgcggtaatacgtaggtggcaagcgttatccggatttattgggcgtaaagcgagcgcaggcggttgcttaggtctgatgtgaaagccttcggcttaaccgaagaagggcatcggaaaccgggcgacttgagtg

SEQ ID NO: 35 - DNA, 142 bp, Ureaplasma parvum
  misc_feature 1..142: note = Ureaplasma sp.2, Ureaplasma parvum (16S rRNA V4 region)
  source 1..142: mol_type = other DNA
gccagcagccgcggtaatacataggatgcaagcgttatccggatttactgggcgtaaaacgagcgcaggcgggtttgtaagtttggtattaaatctagatgcttaacgtctagctgtatcaaaaactgtaaacctagagtgt